(19) United States
(12) Patent Application Publication
(10) Pub. No.: US 2017/0239355 A1
(43) Pub. Date: Aug. 24, 2017

(54) USE OF TRYPTOPHAN DERIVATIVES FOR PROTEIN FORMULATIONS

(71) Applicant: Genentech, Inc., South San Francisco, CA (US)

(72) Inventors: Vikas SHARMA, South San Francisco, CA (US); Danielle LEISKE, South San Francisco, CA (US); Lydia HAMBURG, South San Francisco, CA (US); Cleo SALISBURY, South San Francisco, CA (US)

(21) Appl. No.: 15/393,143

(22) Filed: Dec. 28, 2016

Related U.S. Application Data
(60) Provisional application No. 62/273,273, filed on Dec. 30, 2015.
(60) Provisional application No. 62/321,636, filed on Apr. 12, 2016.

(51) Int. Cl. A61K 47/22; G01N 33/68; B01D 15/32; G06F 19/24; G01N 30/88; C07K 16/24; G01N 33/15

(52) U.S. Cl. CPC A61K 47/22; C07K 16/241; G01N 33/6812; G01N 33/15; G06F 19/24; G01N 30/88; B01D 15/325; C07K 2317/24; C07K 2317/21; C07K 2317/31; C07K 2317/94; G01N 2440/00; G01N 2030/884

(57) ABSTRACT

The invention provides methods and formulations comprising a protein comprising solvent accessible amino acid residues susceptible to oxidation wherein N-acetyl tryptophan (NAT) is used to prevent oxidation of the protein. The invention also provides methods for making such formulations and methods of using such formulations. Methods to measure degradation of NAT in protein formulations are also provided.

### CROSS-REFERENCE TO RELATED APPLICATIONS

This application claims the benefit of U.S. Provisional Application No. 62/273,273, filed Dec. 30, 2015 and U.S. Provisional Application No. 62/321,636, filed Apr. 12, 2016, the contents of each of which are hereby incorporated by reference in its entirety.

### FIELD OF THE INVENTION

This invention relates to liquid formulations comprising a protein and further comprising N-acetyl-tryptophan, and methods for producing and using the liquid formulations.

### BACKGROUND OF THE INVENTION

Oxidative degradation of amino acid residues is a commonly observed phenomenon in protein pharmaceuticals. A number of amino acid residues are susceptible to oxidation, particularly methionine (Met), cysteine (Cys), histidine (His), tryptophan (Trp), and tyrosine (Tyr) (Li et al., Biotechnology and Bioengineering 48:490-500 (1995)). Oxidation is typically observed when the protein is exposed to hydrogen peroxide, light, metal ions or a combination of these during various processing steps (Li et al., Biotechnology and Bioengineering 48:490-500 (1995)). In particular, proteins exposed to light (Wei, et al., Analytical Chemistry 79(7):2797-2805 (2007)), AAPH or Fenton reagents (Ji et al., J Pharm Sci 98(12):4485-500 (2009)) have shown increased levels of oxidation on tryptophan residues, whereas those exposed to hydrogen peroxide have typically shown only methionine oxidation (Ji et al., J Pharm Sci 98(12):4485-500 (2009)). Light exposure can result in protein oxidation through the formation of reactive oxygen species (ROS) including singlet oxygen, hydrogen peroxide and superoxide (Li et al., Biotechnology and Bioengineering 48:490-500 (1995); Wei, et al., Analytical Chemistry 79(7):2797-2805 (2007); Ji et al., J Pharm Sci 98(12):4485-500 (2009); Frokjaer et al., Nat Rev Drug Discov 4(4):298-306 (2005)), whereas protein oxidation typically occurs via hydroxyl radicals in the Fenton mediated reaction (Prousek et al., Pure and Applied Chemistry 79(12):2325-2338 (2007)) and via alkoxyl peroxides in the AAPH mediated reaction (Werber et al., J Pharm Sci 100(8):3307-15 (2011)). Oxidation of tryptophan leads to a myriad of oxidation products, including hydroxytryptophan, kynurenine (Kyn), and N-formylkynurenine, and has the potential to impact formulation safety and efficacy (Li et al., Biotechnology and Bioengineering 48:490-500 (1995); Ji et al., J Pharm Sci 98(12):4485-500 (2009); Frokjaer et al., Nat Rev Drug Discov 4(4):298-306 (2005)). Oxidation of a particular tryptophan residue in the heavy chain complementarity determining region (CDR) of a monoclonal antibody that correlated to loss of biological function has been reported (Wei, et al., Analytical Chemistry 79(7):2797-2805 (2007)). Trp oxidation mediated by a histidine coordinated metal ion has recently been reported for a Fab molecule (Lam et al., Pharm Res 28(10):2543-55 (2011)). Autoxidation of polysorbate 20 in the Fab formulation, leading to the generation of various peroxides, has also been reported in the same study. Autoxidation-induced generation of these peroxides can also lead to methionine oxidation in a protein during long-term storage since Met residues in proteins have been suggested to act as internal antioxidants (Levine et al., Proceedings of the National Academy of Sciences of the United States of America 93(26):15036-15040 (1996)) and are easily oxidized by peroxides. Oxidation of amino acid residues of a protein has the potential to impact its biological activity. This may be especially true for monoclonal antibodies (mAbs). Methionine oxidation at Met254 and Met430 in an IgG1 mAb potentially impacts serum half-life in transgenic mice (Wang et al., Molecular Immunology 48(6-7):860-866 (2011)) and also impacts binding of human IgG1 to FcRn and Fc-gamma receptors (Bertolotti-Ciarlet et al., Molecular Immunology 46(8-9)1878-82 (2009)).

The stability of proteins, especially in liquid state, needs to be evaluated during drug product manufacturing and storage. The development of pharmaceutical formulations sometimes includes addition of antioxidants to prevent oxidation of the active ingredient. Addition of L-methionine to formulations has resulted in reduction of methionine residue oxidation in proteins and peptides (Ji et al., J Pharm Sci 98(12):4485-500 (2009); Lam et al., Journal of Pharmaceutical Sciences 86(11):1250-1255 (1997)). Likewise, addition of L-tryptophan has been shown to reduce oxidation of tryptophan residues (Ji et al., J Pharm Sci 98(12):4485-500 (2009); Lam et al., Pharm Res 28(10):2543-55 (2011)). L-Trp, however, possesses strong absorbance in the UV region (260-290 nm) making it a primary target during photo-oxidation (Creed, D., Photochemistry and Photobiology 39(4):537-562 (1984)). Trp has been hypothesized as an endogenous photosensitizer enhancing the oxygen dependent photo-oxidation of tyrosine (Babu et al., Indian J Biochem Biophys 29(3):296-8 (1992)) and other amino acids (Bent et al., Journal of the American Chemical Society 97(10):2612-2619 (1975)). It has been demonstrated that L-Trp can generate hydrogen peroxide when exposed to light and that L-Trp under UV light produces hydrogen peroxide via the superoxide anion (McCormick et al., Science 191(4226):468-9 (1976); Wentworth et al., Science 293(5536):1806-11 (2001); McCormick et al., Journal of the American Chemical Society 100:312-313 (1978)). Additionally, tryptophan is known to produce singlet oxygen upon exposure to light (Davies, M. J., Biochem Biophys Res Commun 305(3):761-70 (2003)). Similar to the protein oxidation induced by autoxidation of polysorbate 20, it is possible that protein oxidation can occur upon ROS generation by other excipients in the protein formulation (e.g. L-Trp) under normal handling conditions.

The susceptibility for oxidation of a particular protein residue in a liquid formulation may depend on the accessibility of the residue to oxidizing agents (e.g. ROS) in the formulation. Solvent-accessible surface area (SASA) is a measure of the surface area of a biomolecule (e.g. amino acid residue) that is accessible to a solvent. The SASA of an amino acid residue in a protein may indicate the availability of the residue for oxidation. SASA can be calculated using various methods including the Shrake-Rupley algorithm, the linear combinations of pairwise overlaps (LCPO) method, and the power diagram method (Shrake, A & Rupley, J A., J. Mol. Biol. 79(2):351-371, 1973; Weiser et al., J. Comp. Chem. 20(2):217-230, 1999; Klenin et al., J. Comp. Chem. 32(12):2647-2653, 2011). More recently, all-atom molecular dynamics (MD) simulations have been used to calculate SASA for amino acid residues, and a binary dependence on % SASA and liability of Trp oxidation was demonstrated (Sharma, V. et al., PNAS. 111(52):18601-18606, 2014). SASA could therefore be a useful parameter for determining the suitability of including antioxidants in a given protein formulation.

It is apparent from recent studies that the addition of standard excipients, such as L-Trp and polysorbates, to protein compositions that are meant to stabilize the protein can result in unexpected and undesired consequences such as ROS-induced oxidation of the protein. This is of particular concern for protein compositions having oxidation-prone residues. Therefore, there remains a need for the identification of alternative excipients for use in protein compositions and the development of such compositions. Examples of the use of tryptophan derivatives in protein formulations are provided by U.S. Patent Publication Nos. 2014/0322203 and 2014/0314778.

The disclosures of all publications, patents, patent applications and published patent applications referred to herein are hereby incorporated herein by reference in their entirety.

### BRIEF SUMMARY OF THE INVENTION

The present invention provides a method of reducing oxidation of a polypeptide in an aqueous formulation comprising adding an amount of N-acetyltryptophan to the formulation that prevents oxidation of the polypeptide, wherein the polypeptide comprises at least one tryptophan residue with a solvent-accessible surface area (SASA) of greater than about 80 Å2. The invention also provides a method of reducing oxidation of a polypeptide in an aqueous formulation comprising adding an amount of N-acetyltryptophan to the formulation that prevents oxidation of the polypeptide, wherein the polypeptide comprises at least one tryptophan residue with a solvent-accessible surface area (SASA) of greater than about 30%. The invention also provides a method of reducing oxidation of a polypeptide in an aqueous formulation comprising determining the SASA values of tryptophan residues in the polypeptide and adding an amount of N-acetyltryptophan to the formulation that prevents oxidation of the polypeptide if at least one tryptophan residue has a solvent-accessible surface area (SASA) of greater than about 80 Å2. In some embodiments, the SASA value of the tryptophan residues in calculated by molecular dynamic simulation.

In some embodiments, the N-acetyltryptophan is added to the formulation to a concentration of about 0.1 mM to about 5 mM. In some embodiments, the N-acetyltryptophan is added to the formulation to a concentration of about 0.1 mM to about 1 mM. In some embodiments, the N-acetyltryptophan is added to the formulation to a concentration about 0.3 mM.

In some embodiments, the oxidation of the polypeptide is reduced by about 50%, 75%, 80%, 85%, 90%, 95% or 99%. In some embodiments, the formulation is stable at about 2° C. to about 8° C. for about 1095 days.

In some embodiments, the protein concentration in the formulation is about 1 mg/mL to about 250 mg/mL. In some embodiments, the formulation has a pH of about 4.5 to about 7.0. In some embodiments, the formulation further comprises one or more excipients selected from the group consisting of a stabilizer, a buffer, a surfactant, and a tonicity agent.

In some embodiments, the formulation is a pharmaceutical formulation suitable for administration to a subject. In some embodiments, the protein is an antibody. In some embodiments, the antibody is a polyclonal antibody, a monoclonal antibody, a humanized antibody, a human antibody, a chimeric antibody, a multispecific antibody, or an antibody fragment.

In some aspects, the invention provides a liquid formulation comprising a polypeptide and an amount of N-acetyltryptophan to prevent oxidation of the polypeptide, wherein the polypeptide has at least one tryptophan residue with a SASA of greater than about 80 Å2. In some embodiments, the N-acetyltryptophan is added to the formulation to a concentration of about 0.1 mM to about 5 mM. In some embodiments, the N-acetyltryptophan is added to the formulation to a concentration of about 0.1 mM to about 1 mM. In some embodiments, the N-acetyltryptophan is added to the formulation to a concentration about 0.3 mM. In some embodiments, the oxidation of the polypeptide is reduced by about 50%, 75%, 80%, 85%, 90%, 95% or 99%. In some embodiments, the formulation is stable at about 2° C. to about 8° C. for about 1065 days.

In some embodiments, the protein concentration in the formulation is about 1 mg/mL to about 250 mg/mL. In some embodiments, the formulation has a pH of about 4.5 to about 7.0. In some embodiments, the formulation further comprises one or more excipients selected from the group consisting of a stabilizer, a buffer, a surfactant, and a tonicity agent.

In some embodiments, the formulation is a pharmaceutical formulation suitable for administration to a subject. In some embodiments, the protein is an antibody. In some embodiments, the antibody is a polyclonal antibody, a monoclonal antibody, a humanized antibody, a human antibody, a chimeric antibody, a multispecific antibody, or an antibody fragment.

In some aspects, the invention provides a method for screening a formulation for reduced oxidation of a polypeptide wherein the polypeptide comprises at least one tryptophan residue with a SASA of greater than about 80 Å2, the method comprising adding an amount of N-acetyltryptophan to an aqueous composition comprising the polypeptide, adding 2,2′-azobis (2-aminopropane) dihydrochloride (AAPH) to the composition, incubating the composition comprising the polypeptide, N-acetyltryptophan and AAPH for about 14 hours at about 40° C., measuring the polypeptide for oxidation of tryptophan residues in the polypeptide, wherein a formulation comprising an amount of N-acetyltryptophan that results in no more than about 20% oxidation of tryptophan residues of the polypeptide is a suitable formulation for reduced oxidation of the polypeptide. In some embodiments, the N-acetyltryptophan and AAPH are incubated for less than about any of 10 hours, 11 hours, 12 hours, 14 hours, 16 hours, 20 hours, or 24 hours. In some embodiments, no more than about any of 15%, 20% 25%, 30%, or 35%, oxidation of tryptophan residues of the polypeptide is a suitable formulation for reduced oxidation of the polypeptide.

In some aspects, the invention provides a method for screening a formulation for reduced oxidation of a polypeptide comprising determining the SASA values of tryptophan residues in the polypeptide, wherein a tryptophan residue with a SASA of greater than about 80 Å2, is subject to oxidation, adding an amount of N-acetyltryptophan to an aqueous composition comprising the polypeptide, adding 2,2′-azobis (2-aminopropane) dihydrochloride (AAPH) to the composition, incubating the composition comprising the polypeptide, N-acetyltryptophan and AAPH for about 14 hours at about 40° C., measuring the polypeptide for oxidation of tryptophan residues in the polypeptide, wherein a formulation comprising an amount of N-acetyltryptophan that results in no more than about 20% oxidation of tryptophan residues of the polypeptide is a suitable formulation for reduced oxidation of the polypeptide.

In some embodiments of the above aspects, the SASA value of the tryptophan residues in calculated by molecular dynamic simulation.

In some aspects the invention provides a kit comprising the liquid formulation of any one of the embodiments described herein. In some aspects, the invention provides an article of manufacture comprising the liquid formulation of any one of the embodiments described herein.

Provided herein are formulations comprising a protein and N-acetyl-tryptophan (NAT), and methods of making and using the formulations.

In some embodiments, the liquid formulation is a pharmaceutical formulation suitable for administration to a subject. In some embodiments, the formulation is aqueous.

In some embodiments, the NAT prevents oxidation of tryptophan in the protein.

In some embodiments, the protein in the formulation is susceptible to oxidation. In some embodiments, tryptophan in the protein is susceptible to oxidation. In some embodiments, the protein is an antibody (e.g., a polyclonal antibody, a monoclonal antibody, a humanized antibody, a human antibody, a chimeric antibody, or antibody fragment). In some embodiments, the protein concentration in the formulation is about 1 mg/mL to about 250 mg/mL.

In some embodiments, the formulation further comprises one or more excipients selected from the group consisting of a stabilizer, a buffer, a surfactant, and a tonicity agent. In some embodiments, the formulation has a pH of about 4.5 to about 7.0.

The invention also provides a method to determine if a polypeptide in a liquid formulation comprises a tryptophan residue susceptible to oxidation, the method comprising calculating one or more molecule descriptors based on the amino acid sequence of the polypeptide for each tryptophan residue in the polypeptide and applying the one or more molecule descriptors to a machine learning algorithm trained on the one or more molecule descriptors to predict tryptophan oxidation, wherein the molecule descriptors include one or more of the following: a) number of aspartic acid sidechain oxygens within 7 Å of tryptophan delta carbon, b) sidechain solvent accessible surface area (SASA), c) delta carbon SASA, d) total positive charge within 7 Å of tryptophan delta carbon, e) backbone SASA, f) tryptophan sidechain angles, g) packing density within 7 Å of tryptophan delta carbon, h) tryptophan backbone angles, i) SASA of pseudo-pi orbitals, j) backbone flexibility, or k) total negative charge within 7 Å of tryptophan delta carbon. In some embodiments, two, three, four, five, six, seven, eight, nine, ten or eleven molecule descriptors are used. In some embodiments, the molecule descriptors comprise the following: a) number of aspartic acid sidechain oxygens within 7 Å of tryptophan delta carbon, b) sidechain solvent accessible surface area (SASA), c) delta carbon SASA, d) total positive charge within 7 Å of tryptophan delta carbon, e) backbone SASA, f) tryptophan sidechain angles, and g) packing density within 7 Å of tryptophan delta carbon. In some embodiments, oxidation of greater than 35% of tryptophan residues at a particular site indicates susceptibility to oxidation. In some embodiments, the protein is an antibody. In some embodiments, the antibody is a polyclonal antibody, a monoclonal antibody, a humanized antibody, a human antibody, a chimeric antibody, a multispecific antibody, or an antibody fragment.

In some embodiments, the machine learning algorithm was trained by matching molecule descriptors from molecular dynamic simulations of polypeptides based on amino acid sequence of the polypeptide with experimental data for each tryptophan residue in the polypeptide. In some embodiments, the one or more molecule descriptors are calculated using a computer.

The invention also provides a method to reduce oxidation of a polypeptide, comprising identifying tryptophan residues susceptible to oxidation according to any one of the embodiments described above comprising a machine learning algorithm, and introducing an amino acid substitution in the polypeptide to replace one or more tryptophan residues susceptible to oxidation with amino acid residues that are not subject to oxidation. In some embodiments, there is provided a method to reduce oxidation of a polypeptide, comprising introducing an amino acid substitution in the polypeptide to replace one or more tryptophan residues susceptible to oxidation, wherein the one or more tryptophan residues susceptible to oxidation was identified by the method according to any one of the embodiments described above comprising a machine learning algorithm. In some embodiments, the tryptophan residue is replaced by an amino acid residue selected from the group consisting of tyrosine, phenylalanine, leucine, isoleucine, alanine, and valine.

The invention also provides a method to reduce oxidation of a polypeptide in an aqueous formulation, comprising determining the presence of one or more tryptophan residues in the polypeptide susceptible to oxidation according to the method of any one of the embodiments described above comprising a computer learning algorithm, and adding an effective amount of an anti-oxidation agent to the aqueous formulation comprising a polypeptide having a one or more tryptophan residues susceptible to oxidation. In some embodiments, there is provided a method to reduce oxidation of a polypeptide in an aqueous formulation, comprising adding an amount of an anti-oxidation agent to the aqueous formulation to prevent oxidation, wherein polypeptide comprises one or more tryptophan residues susceptible to oxidation identified by the method of any one of the embodiments described above comprising a machine learning algorithm. In some embodiments, the anti-oxidation agent is N-acetyltryptophan. In some embodiments, the N-acetyltryptophan is added to the formulation to a concentration of about 0.1 mM to about 5 mM. In some embodiments, the N-acetyltryptophan is added to the formulation to a concentration of about 0.1 mM to about 1 mM. In some embodiments, the N-acetyltryptophan is added to the formulation to a concentration about 0.3 mM. In some embodiments, the oxidation of the polypeptide is reduced by about 50%, 75%, 80%, 85%, 90%, 95% or 99%. In some embodiments, the formulation is stable at about 2° C. to about 8° C. for about 1095 days. In some embodiments, the protein concentration in the formulation is about 1 mg/mL to about 250 mg/mL. In some embodiments, the formulation has a pH of about 4.5 to about 7.0. In some embodiments, the formulation further comprises one or more excipients selected from the group consisting of a stabilizer, a buffer, a surfactant, and a tonicity agent. In some embodiments, the formulation is a pharmaceutical formulation suitable for administration to a subject. In some embodiments, the protein is an antibody. In some embodiments, the antibody is a polyclonal antibody, a monoclonal antibody, a humanized antibody, a human antibody, a chimeric antibody, a multispecific antibody, or an antibody fragment.

The invention also provides a liquid formulation comprising a polypeptide and an amount of N-acetyltryptophan to prevent oxidation of the polypeptide, wherein the polypeptide has at least one tryptophan residue susceptible to oxidation as measured by the method of any one of the embodiments described above comprising a machine learning algorithm. In some embodiments, the N-acetyltryptophan is added to the formulation to a concentration of about 0.1 mM to about 5 mM. In some embodiments, the N-acetyltryptophan is added to the formulation to a concentration of about 0.1 mM to about 1 mM. In some embodiments, the N-acetyltryptophan is added to the formulation to a concentration about 0.3 mM. In some embodiments, the oxidation of the polypeptide is reduced by about 50%, 75%, 80%, 85%, 90%, 95% or 99%. In some embodiments, the formulation is stable at about 2° C. to about 8° C. for about 1065 days. In some embodiments, the protein concentration in the formulation is about 1 mg/mL to about 250 mg/mL. In some embodiments, the formulation has a pH of about 4.5 to about 7.0. In some embodiments, the formulation further comprises one or more excipients selected from the group consisting of a stabilizer, a buffer, a surfactant, and a tonicity agent. In some embodiments, the formulation is a pharmaceutical formulation suitable for administration to a subject. In some embodiments, the protein is an antibody. In some embodiments, the antibody is a polyclonal antibody, a monoclonal antibody, a humanized antibody, a human antibody, a chimeric antibody, a multispecific antibody, or an antibody fragment. In some embodiments, there is provided a kit comprising the liquid formulation. In some embodiments, there is provided an article of manufacture comprising the liquid formulation.

The invention also provides a method for screening a formulation for reduced oxidation of a polypeptide wherein the polypeptide comprises at least one tryptophan susceptible to oxidation identified by the method of any one of the embodiments described above comprising a machine learning algorithm, the method comprising adding an amount of N-acetyltryptophan to an aqueous composition comprising the polypeptide, adding 2,2′-azobis (2-aminopropane) dihydrochloride (AAPH) to the composition, incubating the composition comprising the polypeptide, N-acetyltryptophan and AAPH for about 14 hours at about 40° C., measuring the polypeptide for oxidation of tryptophan residues in the polypeptide, wherein a formulation comprising an amount of N-acetyltryptophan that results in no more than about 20% oxidation of tryptophan residues of the polypeptide is a suitable formulation for reduced oxidation of the polypeptide. In some embodiments, there is provided a method for screening a formulation for reduced oxidation of a polypeptide comprising a) identifying a polypeptide comprising one or more tryptophan residues susceptible to oxidation by the method of any one of the embodiments described above comprising a machine learning algorithm, b) adding an amount of N-acetyltryptophan to an aqueous composition comprising the polypeptide identified in step a), c) adding 2,2′-azobis (2-aminopropane) dihydrochloride (AAPH) to the composition, d) incubating the composition comprising the polypeptide, N-acetyltryptophan and AAPH for about 14 hours at about 40° C., e) measuring the polypeptide for oxidation of tryptophan residues in the polypeptide, wherein a formulation comprising an amount of N-acetyltryptophan that results in no more than about 20% oxidation of tryptophan residues of the polypeptide is a suitable formulation for reduced oxidation of the polypeptide.

In some aspects, the invention provides methods for measuring N-acetyl tryptophan (NAT) degradation in a composition comprising N-acetyl tryptophan, the method comprising a) applying the composition to a reverse phase chromatography material, wherein the composition is loaded onto the chromatography material that has been equilibrated in a solution comprising a mobile phase A and a mobile phase B, wherein mobile phase A comprises acid in water and mobile phase B comprises acid in acetonitrile, b) eluting the composition from the reverse phase chromatography material with a solution comprising mobile phase A and mobile phase B wherein the ratio of mobile phase B to mobile phase A is increased compared to step a), wherein NAT degradants elute from the chromatography separately from intact NAT, c) quantifying the NAT degradants and the intact NAT. In some embodiments, the ratio of mobile phase B to mobile phase A in step a) is about 2:98. In some embodiments, the ratio of mobile phase B to mobile phase A in step b) increases linearly. In some embodiments, the ratio of mobile phase B to mobile phase A in step b) increases stepwise. In some embodiments, the flow rate of the chromatography is about 1.0 mL/minute. In some embodiments, the ratio of mobile phase B to mobile phase A is increased to about 30:70. In some embodiments, the ratio of mobile phase B to mobile phase A is increased to about 30:70 in about 16 minutes. In some embodiments, the ratio of mobile phase B to mobile phase A is further increased to about 90:70. In some embodiments, the ratio of mobile phase B to mobile phase A is further increased to about 90:70 in about 18.1 minutes. In some embodiments, the ratio of mobile phase B to mobile phase A is increased to about 26:74. In some embodiments, the ratio of mobile phase B to mobile phase A is increased to about 26:74 in about 14 minutes. In some embodiments, the ratio of mobile phase B to mobile phase A is further increased to about 90:70. In some embodiments, the ratio of mobile phase B to mobile phase A is further increased to about 90:70 in about 16.5 minutes. In some embodiments, mobile phase A comprises about 0.1% acid in water. In some embodiments, mobile phase B comprises about 0.1% acid in acetonitrile. In some embodiments, the acid is formic acid. In some embodiments, the reverse phase chromatography material comprises a C18 moiety. In some embodiments, the reverse phase chromatography material comprises a solid support. In some embodiments, the solid support comprises silica. In some embodiments, the reverse phase chromatography material is contained in a column. In some embodiments, the reverse phase chromatography material is a high performance liquid chromatography (HPLC) material or an ultra-high performance liquid chromatography (UPLC) material. In some embodiments, NAT and NAT degradation products are detected by absorbance at 240 nm. In some embodiments, NAT degradation products are identified by mass spectrometry. In some embodiments, the concentration of NAT in the composition is about 10 nM to about 1 mM. In some embodiments, NAT degradation products include one or more of N-Ac-(H, 1,2,3,3a,8,8a-hexahydro-3a-hydroxypyrrolo [2,3-b]-indole 2-carboxylic acid) (N-Ac-PIC), N-Ac-oxyindolylalanine (N-Ac-Oia), N-Ac-N-formyl-kynurenine (N-Ac-NFK), N-Ac-kynurenine (N-Ac-Kyn) and N-Ac-2a,8a-dihydroxy-PIC.

In some aspects, the invention provides methods for measuring N-acetyl tryptophan (NAT) degradation in a composition comprising N-acetyl tryptophan and a polypeptide, the method comprising a) diluting the composition with about 8 M guanidine, b) removing the polypeptide from the composition, c) applying the composition to a reverse phase chromatography material, wherein the composition is loaded onto the chromatography material that has been equilibrated in a solution comprising a mobile phase A and a mobile phase B, wherein mobile phase A comprises acid in water and mobile phase B comprises acid in acetonitrile, d) eluting the composition from the reverse phase chromatography material with a solution comprising mobile phase A and mobile phase B wherein the ratio of mobile phase B to mobile phase A is increased compared to step a), wherein NAT degradants elute from the chromatography separately from intact NAT, e) quantifying the NAT degradants and the intact NAT. In some embodiments, the composition is diluted in about 8M guanidine such that the final concentration of NAT in the composition ranges from about 0.05 mM to about 0.2 mM. In some embodiments, the composition is diluted in about 8M guanidine such that the final concentration of polypeptide in the composition is less than or equal to about 25 mg/mL. In some embodiments, the polypeptide is removed from the composition by filtration. In some embodiments, the filtration uses a filtration membrane with a molecular weight cut-off of about 30 kDal. In some embodiments, the ratio of mobile phase B to mobile phase A in step a) is about 2:98. In some embodiments, the ratio of mobile phase B to mobile phase A in step b) increases linearly. In some embodiments, the ratio of mobile phase B to mobile phase A in step b) increases stepwise. In some embodiments, the flow rate of the chromatography is about 1.0 mL/minute. In some embodiments, the ratio of mobile phase B to mobile phase A is increased to about 30:70. In some embodiments, the ratio of mobile phase B to mobile phase A is increased to about 30:70 in about 16 minutes. In some embodiments, the ratio of mobile phase B to mobile phase A is further increased to about 90:70. In some embodiments, the ratio of mobile phase B to mobile phase A is further increased to about 90:70 in about 18.1 minutes. In some embodiments, the ratio of mobile phase B to mobile phase A is increased to about 26:74. In some embodiments, the ratio of mobile phase B to mobile phase A is increased to about 26:74 in about 14 minutes. In some embodiments, the ratio of mobile phase B to mobile phase A is further increased to about 90:70. In some embodiments, the ratio of mobile phase B to mobile phase A is further increased to about 90:70 in about 16.5 minutes. In some embodiments, mobile phase A comprises about 0.1% acid in water. In some embodiments, mobile phase B comprises about 0.1% acid in acetonitrile. In some embodiments, the acid is formic acid. In some embodiments, the reverse phase chromatography material comprises a C18 moiety. In some embodiments, the reverse phase chromatography material comprises a solid support. In some embodiments, the solid support comprises silica. In some embodiments, the reverse phase chromatography material is contained in a column. In some embodiments, the reverse phase chromatography material is a high performance liquid chromatography (HPLC) material or an ultra-high performance liquid chromatography (UPLC) material. In some embodiments, NAT and NAT degradation products are detected by absorbance at 240 nm. In some embodiments, NAT degradation products are identified by mass spectrometry. In some embodiments, the concentration of NAT in the composition is about 0.1 mM to about 5 mM. In some embodiments, the concentration of NAT in the composition is about 0.3 mM. In some embodiments, NAT degradation products include one or more of N-Ac-PIC, N-Ac-Oia, N-Ac-NFK, N-Ac-Kyn and N-Ac-2a,8a-dihydroxy-PIC.

In some embodiments of the above aspects, the protein concentration in the formulation is about 1 mg/mL to about 250 mg/mL. In some embodiments, the formulation has a pH of about 4.5 to about 7.0. In some embodiments, the formulation further comprises one or more excipients selected from the group consisting of a stabilizer, a buffer, a surfactant, and a tonicity agent. In some embodiments, the formulation is a pharmaceutical formulation suitable for administration to a subject. In some embodiments, the polypeptide is an antibody. In some embodiments, the antibody is a polyclonal antibody, a monoclonal antibody, a humanized antibody, a human antibody, a chimeric antibody, a multispecific antibody or antibody fragment.

In some aspects, the invention provides methods to monitor degradation of NAT in a composition comprising measuring the degradation of NAT in a sample of the composition according to the methods of any one of claims 74-134, wherein the method is repeated one or more times. In some embodiments, the method is repeated every month, every two months, every four months or every six months.

In some aspects, the invention provides a quality assay for a pharmaceutical composition, the quality assay comprising measuring degradation of NAT in a sample of the pharmaceutical composition according to the methods of any one of claims 74-134, wherein the amount of NAT degradants measured in the composition determines if the pharmaceutical composition is suitable for administration to an animal. In some embodiments, an amount of NAT degradants in the pharmaceutical composition of less than about 10 ppm indicates that the pharmaceutical composition is suitable for administration to the animal.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWING

FIG. 1 shows the protection from AAPH stress-induced oxidation of various tryptophan residues by NAT in proteins Mab2, Mab4, Mab1, and Mab6. Both graphs present the same data, differing in x-axis scale. The legend includes the in silico-calculated solvent-accessible surface area for each residue tested.

FIGS. 2A and 2B show the relationship between tryptophan oxidation by AAPH and % sidechain SASA. FIG. 2A shows results from a data set including 38 IgG1 mAbs. FIG. 2B shows results from a data set including 121 mAbs across diverse frameworks including IgG1, IgG2, IgG4, and murine.

FIG. 3 shows random decision forest accuracy as a function of the number of estimators used during training.

FIG. 4 shows random decision forest accuracy as a function of the number of features considered during training.

FIG. 5 shows random decision forest accuracy as a function of the tree depth used during training.

FIG. 6 shows the feature importance (gini importance) of the 14 most relevant simulation-based molecule descriptors for an optimized random decision forest. Training parameters included: 5000 estimators, 3 features considered per node, and a tree depth of 10.

FIG. 7 shows potential degradants of NAT (b series), along with the corresponding Trp degradants (a series).

FIG. 8A shows reverse phase chromatograms 0.2 mM NAT after subjection to different stress conditions. Starred peaks represent peaks only observed under ICH light stress. FIG. 8B shows comparison of fluorescence and absorbance across wavelengths for NAT and NAT degradants in an AAPH stressed sample. The profiles have been normalized such that the NAT peak is set to 1 AU. Note that the only NAT degradant with measurable fluorescence (excitation wavelength=240 nm, emission wavelength=342 nm) is peak 4 (assigned as N-Ac-PIC, based on this data and the MS fragmentation data in FIG. 15E).

FIG. 9 shows retention time alignment of synthetic NAT standards with AAPH induced NAT degradant.

FIG. 10 shows the effect of co-formulation of 5 mM Met on total NAT oxidation (in both histidine and non-histidine containing formulations). Standard deviations of duplicate injections are shown.

FIG. 11 shows the impact of protein on AAPH-induced NAT degradation. A histidine-based buffer containing 0.3 mM NAT with or without 1 mg/ml (0.0067 mM) protein was subjected to AAPH stress. The distribution and levels of NAT degradants are largely independent of the presence of protein. Standard deviations of duplicate injections are shown.

FIG. 12 shows comparison of NAT degradation in protein1 stability samples and AAPH stress model. The inset shows an enlarged view of the indicated area.

FIG. 13 shows linearity of NAT UV-HPLC response (at 240 nm).

FIG. 14 depicts that NAT degradants show a linear response in a 1-20× fold dilution series of the AAPH stressed NAT in His buffer sample. All peaks were detected at 240 nm.

FIGS. 15A-15F show Mass Spec Fragmentation analysis (literature reports for fragmentation in Todorovski, T., M. Fedorova, and R. Hoffmann, Mass spectrometric characterization of peptides containing different oxidized tryptophan residues. J Mass Spectrom, 2011. 46(10): p. 1030-8 and references therein). FIG. 15A shows that Peak 2 and 3 MS data supports identification as N-Ac-Oia diastereomers. Starred fragment is characteristic of Oia.

FIG. 15B shows that Peak 6 MS data supports identification as N-Ac-Kyn. Starred fragment characteristic of kynurenine-containing molecules. FIG. 15C shows that Peak 5 MS data supports identification as N-Ac-NFK. Starred fragment characteristic of kynurenine-containing molecules. FIG. 15D shows that 263.1 ion in Peak group 1 and Peak 4 have similar MS fragmentation patterns and neither are N-Ac-HTP. Starred fragment characteristic of 5-HTP.

FIG. 15E shows that the fragmentation pattern in peak 4 is consistent with potential N-Ac-PIC fragmentation and reported in literature (Fang, L., R. Parti, and P. Hu, Characterization of N-acetyltryptophan degradation products in concentrated human serum albumin solutions and development of an automated high performance liquid chromatography-mass spectrometry method for their quantitation. J Chromatogr A, 2011. 1218(41): p. 7316-24). The tentative assignment is strengthened based on fluorescence data shown in FIG. 8B. FIG. 15F shows that the doubly oxidized species in Peak Group 1 are likely N-Ac-DiOia or N-Ac-3a, 8a-dihydroxy-PIC.

### DETAILED DESCRIPTION

In some aspects, the invention provides methods of reducing oxidation of a polypeptide in an aqueous formulation comprising adding an amount of N-acetyltryptophan to the formulation that prevents oxidation of the polypeptide, wherein the polypeptide comprises at least one tryptophan residue with a solvent-accessible surface area (SASA) of greater than about 80 Å². In some aspects, the invention provides methods of reducing oxidation of a polypeptide in an aqueous formulation comprising adding an amount of N-acetyltryptophan to the formulation that prevents oxidation of the polypeptide, wherein the polypeptide comprises at least one tryptophan residue with a solvent-accessible surface area (SASA) of greater than about 30%. In some aspects, the invention provides methods of reducing oxidation of a polypeptide in an aqueous formulation comprising determining the SASA values of tryptophan residues in the polypeptide and adding an amount of N-acetyltryptophan to the formulation that prevents oxidation of the polypeptide if at least one tryptophan residue has a solvent-accessible surface area (SASA) of greater than about 80 Å².

In some aspects, the invention provides methods for screening a formulation for reduced oxidation of a polypeptide wherein the polypeptide comprises at least one tryptophan residue with a SASA of greater than about 80 Å², wherein a formulation comprising an amount of N-acetyltryptophan that results in no more than about 20% oxidation of tryptophan residues of the polypeptide is a suitable formulation for reduced oxidation of the polypeptide. In some aspects the invention provides methods for screening a formulation for reduced oxidation of a polypeptide comprising determining the SASA values of tryptophan residues in the polypeptide, wherein a tryptophan residue with a SASA of greater than about 80 Å², is subject to oxidation, wherein a formulation comprising an amount of N-acetyltryptophan that results in no more than about 20% oxidation of tryptophan residues of the polypeptide is a suitable formulation for reduced oxidation of the polypeptide.

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile.

A “sterile” formulation is aseptic or free or essentially free from all living microorganisms and their spores.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected amount of light exposure and/or temperature for a selected time period. Stability can be evaluated qualitatively and/or quantitatively in a variety of different ways, including evaluation of aggregate formation (for example using size exclusion chromatography, by measuring turbidity, and/or by visual inspection); evaluation of ROS formation (for example by using a light stress assay or a 2,2′-Azobis(2-Amidinopropane) Dihydrochloride (AAPH) stress assay); oxidation of specific amino acid residues of the protein (for example a Trp residue and/or a Met residue of a monoclonal antibody); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or target binding function of the protein (e.g., antigen binding function of an antibody); etc. Instability may involve any one or more of: aggregation, deamidation (e.g. Asn deamidation), oxidation (e.g. Met oxidation and/or Trp oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc.

A protein “retains its physical stability” in a pharmaceutical formulation if it shows no signs or very little of aggregation, precipitation, fragmentation, and/or denaturation upon visual examination of color and/or clarity, or as measured by UV light scattering or by size exclusion chromatography.

A protein “retains its chemical stability” in a pharmaceutical formulation, if the chemical stability at a given time is such that the protein is considered to still retain its biological activity as defined below. Chemical stability can be assessed by detecting and quantifying chemically altered forms of the protein. Chemical alteration may involve protein oxidation which can be evaluated using tryptic peptide mapping, reverse-phase high-performance liquid chromatography (HPLC) and liquid chromatography-mass spectrometry (LC/MS), for example. Other types of chemical alteration include charge alteration of the protein which can be evaluated by ion-exchange chromatography or icIEF, for example.

A protein “retains its biological activity” in a pharmaceutical formulation, if the biological activity of the protein at a given time is within about 20% (such as within about 10%) of the biological activity exhibited at the time the pharmaceutical formulation was prepared (within the errors of the assay), as determined for example in an antigen binding assay for a monoclonal antibody.

As used herein, “biological activity” of a protein refers to the ability of the protein to bind its target, for example the ability of a monoclonal antibody to bind to an antigen. It can further include a biological response which can be measured in vitro or in vivo. Such activity may be antagonistic or agonistic.

A protein which is “susceptible to oxidation” is one comprising one or more residue(s) that has been found to be prone to oxidation such as, but not limited to, methionine (Met), cysteine (Cys), histidine (His), tryptophan (Trp), and tyrosine (Tyr). For example, a tryptophan amino acid in the Fab portion of a monoclonal antibody or a methionine amino acid in the Fc portion of a monoclonal antibody may be susceptible to oxidation.

An “oxidation labile” residue of a protein is a residue having greater than 35% oxidation in an oxidation assay (e.g. AAPH-induced or thermal-induced oxidation). The percent oxidation of a residue in a protein can be determined by any method known in the art, such as tryptic digest followed by LC-MS/MS for site-specific Trp oxidation.

A “solvent-accessible surface area” or “SASA” of a biomolecule in a solvent is the surface area of the biomolecule that is accessible to the solvent. SASA can be expressed in units of measurement (e.g., square Ångstroms) or as a percentage of the surface area that is accessible to the solvent. For example, the SASA of an amino acid residue in a polypeptide can be 80 Å², or 30%. SASA can be determined by any method known in the art, including the Shrake-Rupley algorithm, the LCPO method, the power diagram method, or molecular dynamics simulations.

By “isotonic” is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example.

As used herein, “buffer” refers to a buffered solution that resists changes in pH by the action of its acid-base conjugate components. The buffer of this invention preferably has a pH in the range from about 4.5 to about 8.0. For example, histidine acetate is an example of a buffer that will control the pH in this range.

A “preservative” is a compound which can be optionally included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. In one embodiment, the preservative herein is benzyl alcohol.

As used herein, a “surfactant” refers to a surface-active agent, preferably a nonionic surfactant. Examples of surfactants herein include polysorbate (for example, polysorbate 20 and, polysorbate 80); poloxamer (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.); polyethyl glycol, polypropyl glycol, and copolymers of ethylene and propylene glycol (e.g. Pluronics, PF68 etc); etc. In one embodiment, the surfactant herein is polysorbate 20. In yet another embodiment, the surfactant herein is poloxamer 188.

“Pharmaceutically acceptable” excipients or carriers as used herein include pharmaceutically acceptable carriers, stabilizers, buffers, acids, bases, sugars, preservatives, surfactants, tonicity agents, and the like, which are well known in the art (Remington: The Science and Practice of Pharmacy, 22^{nd} Ed., Pharmaceutical Press, 2012). Examples of pharmaceutically acceptable excipients include buffers such as phosphate, citrate, acetate, and other organic acids; antioxidants including ascorbic acid, L-tryptophan and methionine; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; metal complexes such as Zn-protein complexes; chelating agents such as EDTA; sugar alcohols such as mannitol or sorbitol; salt-forming counterions such as sodium; and/or nonionic surfactants such as polysorbate, poloxamer, polyethylene glycol (PEG), and PLURONICS™. “Pharmaceutically acceptable” excipients or carriers are those which can reasonably be administered to a subject to provide an effective dose of the active ingredient employed and that are nontoxic to the subject being exposed thereto at the dosages and concentrations employed.

The protein which is formulated is preferably essentially pure and desirably essentially homogeneous (e.g., free from contaminating proteins etc.). “Essentially pure” protein means a composition comprising at least about 90% by weight of the protein (e.g., monoclonal antibody), based on total weight of the composition, preferably at least about 95% by weight. “Essentially homogeneous” protein means a composition comprising at least about 99% by weight of the protein (e.g., monoclonal antibody), based on total weight of the composition.

The terms “protein” “polypeptide” and “peptide” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, proteins containing one or more analogs of an amino acid (including, for example, unnatural amino acids, etc.), as well as other modifications known in the art. Examples of proteins encompassed within the definition herein include mammalian proteins, such as, e.g., renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; leptin; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; a tumor necrosis factor receptor such as death receptor 5 and CD120; TNF-related apoptosis-inducing ligand (TRAIL); B-cell maturation antigen (BCMA); B-lymphocyte stimulator (BLyS); a proliferation-inducing ligand (APRIL); enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; platelet-derived endothelial cell growth factor (PD-ECGF); a vascular endothelial growth factor family protein (e.g., VEGF-A, VEGF-B, VEGF-C, VEGF-D, and P1GF); a platelet-derived growth factor (PDGF) family protein (e.g., PDGF-A, PDGF-B, PDGF-C, PDGF-D, and dimers thereof); fibroblast growth factor (FGF) family such as aFGF, bFGF, FGF4, and FGF9; epidermal growth factor (EGF); receptors for hormones or growth factors such as a VEGF receptor(s) (e.g., VEGFR1, VEGFR2, and VEGFR3), epidermal growth factor (EGF) receptor(s) (e.g., ErbB1, ErbB2, ErbB3, and ErbB4 receptor), platelet-derived growth factor (PDGF) receptor(s) (e.g., PDGFR-α and PDGFR-β), and fibroblast growth factor receptor(s); TIE ligands (Angiopoietins, ANGPT1, ANGPT2); Angiopoietin receptor such as TIE1 and TIE2; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-b; transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins (IGFBPs); CD proteins such as CD3, CD4, CD8, CD19 and CD20; erythropoietin; osteoinductive factors; immunotoxins; a bone morphogenetic protein (BMP); a chemokine such as CXCL12 and CXCR4; an interferon such as interferon-alpha, -beta, and -gamma; colony stimulating factors (CSFs), e.g., M-CSF, GM-CSF, and G-CSF; a cytokine such as interleukins (ILs), e.g., IL-1 to IL-10; midkine; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; ephrins; Bv8; Delta-like ligand 4 (DLL4); Del-1; BMP9; BMP10; Follistatin; Hepatocyte growth factor (HGF)/scatter factor (SF); Alk1; Robo4; ESM1; Perlecan; EGF-like domain, multiple 7 (EGFL7); CTGF and members of its family; thrombospondins such as thrombospondin1 and thrombospondin2; collagens such as collagen IV and collagen XVIII; neuropilins such as NRP1 and NRP2; Pleiotrophin (PTN); Progranulin; Proliferin; Notch proteins such as Notchl and Notch4; semaphorins such as Sema3A, Sema3C, and Sema3F; a tumor associated antigen such as CA125 (ovarian cancer antigen); immunoadhesins; and fragments and/or variants of any of the above-listed proteins as well as antibodies, including antibody fragments, binding to one or more protein, including, for example, any of the above-listed proteins.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

An “isolated” protein (e.g., an isolated antibody) is one which has been identified and separated and/or recovered from a component of its natural environment. Contaminant components of its natural environment are materials which would interfere with research, diagnostic or therapeutic uses for the protein, and may include enzymes, hormones, and other proteinaceous or nonproteinaceous solutes. Isolated protein includes the protein in situ within recombinant cells since at least one component of the protein's natural environment will not be present. Ordinarily, however, isolated protein will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the C_{H}1, C_{H}2 and C_{H}3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “V_{H}.” The variable domain of the light chain may be referred to as “V_{L}.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“κ”) and lambda (“λ”), based on the amino acid sequences of their constant domains.

The term IgG “isotype” or “subclass” as used herein is meant any of the subclasses of immunoglobulins defined by the chemical and antigenic characteristics of their constant regions. Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed., W.B. Saunders, Co., 2000. An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, in The Pharmacology of Monoclonal Antibodies, vol. 113, Rosenburg and Moore eds., Springer-Verlag, New York, pp. 269-315, 1994.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996). In some embodiments, the HVRs are Complementarity Determining Regions (CDRs)

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | Numbering) |
| H1 | H31-H35 | H26-H35 | H26-H32 | H30-H35 (Chothia |
| | | | | Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The term “multispecific antibody” is used in the broadest sense and specifically covers an antibody comprising an antigen-binding domain that has polyepitopic specificity (i.e., is capable of specifically binding to two, or more, different epitopes on one biological molecule or is capable of specifically binding to epitopes on two, or more, different biological molecules). In some embodiments, an antigen-binding domain of a multispecific antibody (such as a bispecific antibody) comprises two VH/VL units, wherein a first VH/VL unit specifically binds to a first epitope and a second VH/VL unit specifically binds to a second epitope, wherein each VH/VL unit comprises a heavy chain variable domain (VH) and a light chain variable domain (VL). Such multispecific antibodies include, but are not limited to, full length antibodies, antibodies having two or more VL and VH domains, antibody fragments such as Fab, Fv, dsFv, scFv, diabodies, bispecific diabodies and triabodies, antibody fragments that have been linked covalently or non-covalently. A VH/VL unit that further comprises at least a portion of a heavy chain constant region and/or at least a portion of a light chain constant region may also be referred to as a “hemimer” or “half antibody.” In some embodiments, a half antibody comprises at least a portion of a single heavy chain variable region and at least a portion of a single light chain variable region. In some such embodiments, a bispecific antibody that comprises two half antibodies and binds to two antigens comprises a first half antibody that binds to the first antigen or first epitope but not to the second antigen or second epitope and a second half antibody that binds to the second antigen or second epitope and not to the first antigen or first epitope. According to some embodiments, the multispecific antibody is an IgG antibody that binds to each antigen or epitope with an affinity of 5 M to 0.001 pM, 3 M to 0.001 pM, 1 M to 0.001 pM, 0.5 M to 0.001 pM, or 0.1 M to 0.001 pM. In some embodiments, a hemimer comprises a sufficient portion of a heavy chain variable region to allow intramolecular disulfide bonds to be formed with a second hemimer. In some embodiments, a hemimer comprises a knob mutation or a hole mutation, for example, to allow heterodimerization with a second hemimer or half antibody that comprises a complementary hole mutation or knob mutation. Knob mutations and hole mutations are discussed further below.

A “bispecific antibody” is a multispecific antibody comprising an antigen-binding domain that is capable of specifically binding to two different epitopes on one biological molecule or is capable of specifically binding to epitopes on two different biological molecules. A bispecific antibody may also be referred to herein as having “dual specificity” or as being “dual specific.” Unless otherwise indicated, the order in which the antigens bound by a bispecific antibody are listed in a bispecific antibody name is arbitrary. In some embodiments, a bispecific antibody comprises two half antibodies, wherein each half antibody comprises a single heavy chain variable region and optionally at least a portion of a heavy chain constant region, and a single light chain variable region and optionally at least a portion of a light chain constant region. In certain embodiments, a bispecific antibody comprises two half antibodies, wherein each half antibody comprises a single heavy chain variable region and a single light chain variable region and does not comprise more than one single heavy chain variable region and does not comprise more than one single light chain variable region. In some embodiments, a bispecific antibody comprises two half antibodies, wherein each half antibody comprises a single heavy chain variable region and a single light chain variable region, and wherein the first half antibody binds to a first antigen and not to a second antigen and the second half antibody binds to the second antigen and not to the first antigen.

The term “knob-into-hole” or “KnH” technology as used herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc binding interfaces, CL:CH1 interfaces or VH/VL interfaces of antibodies (see, e.g., US 2011/0287009, US2007/0178552, WO 96/027011, WO 98/050431, and Zhu et al., 1997, Protein Science 6:781-788). In some embodiments, KnHs drive the pairing of two different heavy chains together during the manufacture of multispecific antibodies. For example, multispecific antibodies having KnH in their Fc regions can further comprise single variable domains linked to each Fc region, or further comprise different heavy chain variable domains that pair with similar or different light chain variable domains. KnH technology can also be used to pair two different receptor extracellular domains together or any other polypeptide sequences that comprises different target recognition sequences (e.g., including affibodies, peptibodies and other Fc fusions).

The term “knob mutation” as used herein refers to a mutation that introduces a protuberance (knob) into a polypeptide at an interface in which the polypeptide interacts with another polypeptide. In some embodiments, the other polypeptide has a hole mutation (see e.g., U.S. Pat. No. 5,731,168, U.S. Pat. No. 5,807,706, U.S. Pat. No. 5,821,333, U.S. Pat. No. 7,695,936, U.S. Pat. No. 8,216,805, each incorporated herein by reference in its entirety).

The term “hole mutation” as used herein refers to a mutation that introduces a cavity (hole) into a polypeptide at an interface in which the polypeptide interacts with another polypeptide. In some embodiments, the other polypeptide has a knob mutation (see e.g., U.S. Pat. No. 5,731,168, U.S. Pat. No. 5,807,706, U.S. Pat. No. 5,821,333, U.S. Pat. No. 7,695,936, U.S. Pat. No. 8,216,805, each incorporated herein by reference in its entirety).

The expression “linear antibodies” refers to the antibodies described in Zapata et al. (1995 Protein Eng, 8(10):1057-1062). Briefly, these antibodies comprise a pair of tandem Fd segments (VH—CH1-VH—CH1) which, together with complementary light chain polypeptides, form a pair of antigen binding regions. Linear antibodies can be bispecific or monospecific.

The term “about” as used herein refers to an acceptable error range for the respective value as determined by one of ordinary skill in the art, which will depend in part how the value is measured or determined, i.e., the limitations of the measurement system. For example, “about” can mean within 1 or more than 1 standard deviations, per the practice in the art. A reference to “about” a value or parameter herein includes and describes embodiments that are directed to that value or parameter per se. For example, a description referring to “about X” includes description of “X”.

As used in this specification and the appended claims, the singular forms “a”, “an” and “the” include plural referents unless the content clearly dictates otherwise. Thus, for example, reference to “a compound” optionally includes a combination of two or more such compounds, and the like.

It is understood that aspects and embodiments of the invention described herein include “comprising,” “consisting,” and “consisting essentially of” aspects and embodiments.

### II. Protein Formulations and Preparation

The invention herein relates to formulations (e.g., liquid formulations) comprising a protein and N-acetyl-tryptophan (NAT), wherein the NAT prevents oxidation of the protein. In some embodiments, the protein is susceptible to oxidation. In some embodiments, methionine, cysteine, histidine, tryptophan, and/or tyrosine in the protein is susceptible to oxidation. In some embodiments, tryptophan in the protein is susceptible to oxidation. In some embodiments, the protein comprises at least one tryptophan residue with a solvent-accessible surface area (SASA) greater than about 50 Å² to about 250 Å² (such as greater than about any of 50, 60, 70, 80, 90, 100, 120, 140, 160, 180, 200, 225, or 250 Å², including any ranges between these values). In some embodiments, the SASA is greater than about 80 Å². In some embodiments, the protein comprises at least one tryptophan residue with a SASA greater than about 15% to about 45% (such as greater than about any of 15, 20, 25, 30, 35, 40, or 45%). In some embodiments, the SASA is greater than about 30%. SASA can be calculated using any method known in the art, such as the in silico all-atom molecular dynamics (MD) simulation method described in Sharma, V. et al., PNAS. 111(52):18601-18606, 2014. In some embodiments, SASA of a tryptophan residue is measured at a pH range from about 4.0 to about 8.5. In some embodiments, SASA of a tryptophan residue is measured at a temperature ranging from about 5° C. to about 40° C. In some embodiments, SASA of a tryptophan residue is measured at a salt concentration ranging from about 0 mM to about 500 mM. In some embodiments, SASA of a tryptophan residue is measured at a pH of about 5.0 to about 7.5, a temperature of about 5° C. to about 25° C. and a salt concentration of about 0 mM to about 200 mM. In some embodiments, the protein comprises at least one tryptophan residue predicted to be susceptible to oxidation by a machine learning algorithm trained on associations of tryptophan residue oxidation susceptibility with a plurality of molecule descriptors of the tryptophan residue based on MD simulations. In some embodiments, the formulation further comprises at least one additional protein according to any of the proteins described herein. In some embodiments, the formulation is a liquid formulation. In some embodiments, the formulation is an aqueous formulation.

In some embodiments, the NAT in the formulation is from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values), or up to the highest concentration that the NAT is soluble in the formulation. In some embodiments, the NAT in the formulation is about 1 mM. In some embodiments, the NAT prevents oxidation of one or more tryptophan amino acids in the protein. In some embodiments, the NAT prevents oxidation of the protein by a reactive oxygen species (ROS). In a further embodiment, the reactive oxygen species is selected from the group consisting of a singlet oxygen, a superoxide (O₂—), an alkoxyl radical, a peroxyl radical, a hydrogen peroxide (H₂O₂), a dihydrogen trioxide (H₂O₃), a hydrotrioxy radical (HO₃.), ozone (O₃), a hydroxyl radical, and an alkyl peroxide. For example, a tryptophan amino acid in the Fab portion of a monoclonal antibody and/or a methionine amino acid in the Fc portion of a monoclonal antibody can be susceptible to oxidation.

In some embodiments, the protein (e.g., the antibody) concentration in the formulation is about 1 mg/mL to about 250 mg/mL. In some embodiments, the protein is a therapeutic protein. Exemplary protein concentrations in the formulation include from about 1 mg/mL to more than about 250 mg/mL, from about 1 mg/mL to about 250 mg/mL, from about 10 mg/mL to about 250 mg/mL, from about 15 mg/mL to about 225 mg/mL, from about 20 mg/mL to about 200 mg/mL, from about 25 mg/mL to about 175 mg/mL, from about 25 mg/mL to about 150 mg/mL, from about 25 mg/mL to about 100 mg/mL, from about 30 mg/mL to about 100 mg/mL or from about 45 mg/mL to about 55 mg/mL.

In some embodiments, the protein is an antibody. In some embodiments, the antibody is a polyclonal antibody, a monoclonal antibody, a humanized antibody, a human antibody, a chimeric antibody, a multispecific antibody, or an antibody fragment. In some embodiments, the antibody is derived from an IgG1 antibody sequence. In a further embodiment, the NAT prevents oxidation of one or more amino acids in the Fab portion of an antibody. In another further embodiment, the NAT prevents oxidation of one or more amino acids in the Fc portion of an antibody.

In some embodiments, the formulation is aqueous. In some embodiments, the formulation further comprises one or more excipients selected from the group consisting of a stabilizer, a buffer, a surfactant, and a tonicity agent. For example, a formulation of the invention can comprise a monoclonal antibody, NAT as provided herein which prevents oxidation of the protein, and a buffer that maintains the pH of the formulation to a desirable level. In some embodiments, a formulation provided herein has a pH of about 4.5 to about 9.0. In some embodiments, a formulation provided herein has a pH of about 4.5 to about 7.0. In certain embodiments the pH is in the range from pH 4.0 to 8.5, in the range from pH 4.0 to 8.0, in the range from pH 4.0 to 7.5, in the range from pH 4.0 to 7.0, in the range from pH 4.0 to 6.5, in the range from pH 4.0 to 6.0, in the range from pH 4.0 to 5.5, in the range from pH 4.0 to 5.0, in the range from pH 4.0 to 4.5, in the range from pH 4.5 to 9.0, in the range from pH 5.0 to 9.0, in the range from pH 5.5 to 9.0, in the range from pH 6.0 to 9.0, in the range from pH 6.5 to 9.0, in the range from pH 7.0 to 9.0, in the range from pH 7.5 to 9.0, in the range from pH 8.0 to 9.0, in the range from pH 8.5 to 9.0, in the range from pH 5.7 to 6.8, in the range from pH 5.8 to 6.5, in the range from pH 5.9 to 6.5, in the range from pH 6.0 to 6.5, or in the range from pH 6.2 to 6.5. In certain embodiments of the invention, the formulation has a pH of 6.2 or about 6.2. In certain embodiments of the invention, the formulation has a pH of 6.0 or about 6.0. In some embodiments, the formulation further comprises at least one additional protein according to any of the proteins described herein.

In some embodiments, the formulation provided herein is a pharmaceutical formulation suitable for administration to a subject. As used herein a “subject” or an “individual” for purposes of treatment or administration refers to any animal classified as a mammal, including humans, domestic and farm animals, and zoo, sports, or pet animals, such as dogs, horses, cats, cows, etc. In some embodiments, the mammal is human.

Proteins and antibodies in the formulation may be prepared using methods known in the art. An antibody (e.g., full length antibodies, antibody fragments and multispecific antibodies) in the formulation can be prepared using techniques available in the art, non-limiting exemplary methods of which are described in more detail in the following sections. The methods herein can be adapted by one of skill in the art for the preparation of formulations comprising other proteins such as peptide-based inhibitors. See Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); Short Protocols in Molecular Biology (Ausubel et al., eds., J. Wiley and Sons, 2002); Current Protocols in Protein Science, (Horswill et al., 2006); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010) for generally well understood and commonly employed techniques and procedures for the production of therapeutic proteins, which are all incorporated herein by reference in their entirety.

In some embodiments, according to any of the formulations (e.g., liquid formulations) described above, the formulation comprises two or more proteins (e.g., the formation is a co-formulation of two or more proteins). For example, in some embodiments, the formulation is a co-formulation comprising two or more proteins and N-acetyl-tryptophan (NAT), wherein the NAT prevents oxidation of at least one of the two or more proteins. In some embodiments, the NAT prevents oxidation of a plurality of the two or more proteins. In some embodiments, the NAT prevents oxidation of each of the two or more proteins. In some embodiments, at least one of the two or more proteins comprises at least one tryptophan residue a) with a solvent-accessible surface area (SASA) greater than about 50 Å² to about 250 Å² (such as greater than about any of 50, 60, 70, 80, 90, 100, 120, 140, 160, 180, 200, 225, or 250 Å², including any ranges between these values); b) with a SASA greater than about 15% to about 45% (such as greater than about any of 15, 20, 25, 30, 35, 40, or 45%); or c) predicted to be susceptible to oxidation by a machine learning algorithm trained on associations of tryptophan residue oxidation susceptibility with a plurality of molecule descriptors of the tryptophan residue based on MD simulations. In some embodiments, a plurality of the two or more proteins comprises at least one tryptophan residue a) with a solvent-accessible surface area (SASA) greater than about 50 Å² to about 250 Å² (such as greater than about any of 50, 60, 70, 80, 90, 100, 120, 140, 160, 180, 200, 225, or 250 Å², including any ranges between these values); b) with a SASA greater than about 15% to about 45% (such as greater than about any of 15, 20, 25, 30, 35, 40, or 45%); or c) predicted to be susceptible to oxidation by a machine learning algorithm trained on associations of tryptophan residue oxidation susceptibility with a plurality of molecule descriptors of the tryptophan residue based on MD simulations. In some embodiments, each of the two or more proteins comprises at least one tryptophan residue a) with a solvent-accessible surface area (SASA) greater than about 50 Å² to about 250 Å² (such as greater than about any of 50, 60, 70, 80, 90, 100, 120, 140, 160, 180, 200, 225, or 250 Å², including any ranges between these values); b) with a SASA greater than about 15% to about 45% (such as greater than about any of 15, 20, 25, 30, 35, 40, or 45%); or c) predicted to be susceptible to oxidation by a machine learning algorithm trained on associations of tryptophan residue oxidation susceptibility with a plurality of molecule descriptors of the tryptophan residue based on MD simulations. In some embodiments, at least one of the two or more proteins is an antibody, such as a polyclonal antibody, a monoclonal antibody, a humanized antibody, a human antibody, a chimeric antibody, a multispecific antibody, or an antibody fragment. In some embodiments, a plurality of the two or more proteins are antibodies, such as antibodies independently selected from among a polyclonal antibody, a monoclonal antibody, a humanized antibody, a human antibody, a chimeric antibody, a multispecific antibody, or an antibody fragment. In some embodiments, each of the two or more proteins is an antibody, such as an antibody independently selected from among a polyclonal antibody, a monoclonal antibody, a humanized antibody, a human antibody, a chimeric antibody, a multispecific antibody, or an antibody fragment. In some embodiments, one or more antibodies of the formulation are derived from an IgG1 antibody sequence. In some embodiments, the formulation is a liquid formulation. In some embodiments, the formulation is an aqueous formulation.

A. Antibody Preparation

The antibody in the liquid formulations provided herein is directed against an antigen of interest. Preferably, the antigen is a biologically important polypeptide and administration of the antibody to a mammal suffering from a disorder can result in a therapeutic benefit in that mammal. However, antibodies directed against nonpolypeptide antigens are also contemplated.

Where the antigen is a polypeptide, it may be a transmembrane molecule (e.g. receptor) or ligand such as a growth factor. Exemplary antigens include molecules such as vascular endothelial growth factor (VEGF); CD20; ox-LDL; ox-ApoB100; renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; a tumor necrosis factor receptor such as death receptor 5 and CD120; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des (1-3)-IGF-I (brain IGF-I), insulin-like growth factor binding proteins; CD proteins such as CD3, CD4, CD8, CD19 and CD20; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as HER2, HER3 or HER4 receptor; and fragments of any of the above-listed polypeptides.

(i) Antigen Preparation

Soluble antigens or fragments thereof, optionally conjugated to other molecules, can be used as immunogens for generating antibodies. For transmembrane molecules, such as receptors, fragments of these (e.g. the extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule. Other antigens and forms thereof useful for preparing antibodies will be apparent to those in the art.

(ii) Certain Antibody-Based Methods

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies of interest can be made using the hybridoma method first described by Kohler et al., Nature, 256:495 (1975), and further described, e.g., in Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981), and Ni, Xiandai Mianyixue, 26(4):265-268 (2006) regarding human-human hybridomas. Additional methods include those described, for example, in U.S. Pat. No. 7,189,826 regarding production of monoclonal human natural IgM antibodies from hybridoma cell lines. Human hybridoma technology (Trioma technology) is described in Vollmers and Brandlein, Histology and Histopathology, 20(3):927-937 (2005) and Vollmers and Brandlein, Methods and Findings in Experimental and Clinical Pharmacology, 27(3):185-91 (2005).

For various other hybridoma techniques, see, e.g., US 2006/258841; US 2006/183887 (fully human antibodies), US 2006/059575; US 2005/287149; US 2005/100546; US 2005/026229; and U.S. Pat. Nos. 7,078,492 and 7,153,507. An exemplary protocol for producing monoclonal antibodies using the hybridoma method is described as follows. In one embodiment, a mouse or other appropriate host animal, such as a hamster, is immunized to elicit lymphocytes that produce or are capable of producing antibodies that will specifically bind to the protein used for immunization. Antibodies are raised in animals by multiple subcutaneous (sc) or intraperitoneal (ip) injections of a polypeptide of interest or a fragment thereof, and an adjuvant, such as monophosphoryl lipid A (MPL)/trehalose dicrynomycolate (TDM) (Ribi Immunochem. Research, Inc., Hamilton, Mont.). A polypeptide of interest (e.g., antigen) or a fragment thereof may be prepared using methods well known in the art, such as recombinant methods, some of which are further described herein. Serum from immunized animals is assayed for anti-antigen antibodies, and booster immunizations are optionally administered. Lymphocytes from animals producing anti-antigen antibodies are isolated. Alternatively, lymphocytes may be immunized in vitro.

Lymphocytes are then fused with myeloma cells using a suitable fusing agent, such as polyethylene glycol, to form a hybridoma cell. See, e.g., Goding, Monoclonal Antibodies: Principles and Practice, pp. 59-103 (Academic Press, 1986). Myeloma cells may be used that fuse efficiently, support stable high-level production of antibody by the selected antibody-producing cells, and are sensitive to a medium such as HAT medium. Exemplary myeloma cells include, but are not limited to, murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

The hybridoma cells thus prepared are seeded and grown in a suitable culture medium, e.g., a medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells. Preferably, serum-free hybridoma cell culture methods are used to reduce use of animal-derived serum such as fetal bovine serum, as described, for example, in Even et al., Trends in Biotechnology, 24(3), 105-108 (2006).

Oligopeptides as tools for improving productivity of hybridoma cell cultures are described in Franek, Trends in Monoclonal Antibody Research, 111-122 (2005). Specifically, standard culture media are enriched with certain amino acids (alanine, serine, asparagine, proline), or with protein hydrolysate fractions, and apoptosis may be significantly suppressed by synthetic oligopeptides, constituted of three to six amino acid residues. The peptides are present at millimolar or higher concentrations.

Culture medium in which hybridoma cells are growing may be assayed for production of monoclonal antibodies that bind to an antibody described herein. The binding specificity of monoclonal antibodies produced by hybridoma cells may be determined by immunoprecipitation or by an in vitro binding assay, such as radioimmunoassay (RIA) or enzyme-linked immunoadsorbent assay (ELISA). The binding affinity of the monoclonal antibody can be determined, for example, by Scatchard analysis. See, e.g., Munson et al., Anal. Biochem., 107:220 (1980).

After hybridoma cells are identified that produce antibodies of the desired specificity, affinity, and/or activity, the clones may be subcloned by limiting dilution procedures and grown by standard methods. See, e.g., Goding, supra. Suitable culture media for this purpose include, for example, D-MEM or RPMI-1640 medium. In addition, hybridoma cells may be grown in vivo as ascites tumors in an animal. Monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography. One procedure for isolation of proteins from hybridoma cells is described in US 2005/176122 and U.S. Pat. No. 6,919,436. The method includes using minimal salts, such as lyotropic salts, in the binding process and preferably also using small amounts of organic solvents in the elution process.

(iii) Certain Library Screening Methods

Antibodies in the formulations and compositions described herein can be made by using combinatorial libraries to screen for antibodies with the desired activity or activities. For example, a variety of methods are known in the art for generating phage display libraries and screening such libraries for antibodies possessing the desired binding characteristics. Such methods are described generally in Hoogenboom et al. in Methods in Molecular Biology 178:1-37 (O'Brien et al., ed., Human Press, Totowa, N.J., 2001). For example, one method of generating antibodies of interest is through the use of a phage antibody library as described in Lee et al., J. Mol. Biol. (2004), 340(5):1073-93.

In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the antibodies can be obtained by designing a suitable antigen screening procedure to select for the phage clone of interest followed by construction of a full length antibody clone using the Fv sequences from the phage clone of interest and suitable constant region (Fc) sequences described in Kabat et al., Sequences of Proteins of Immunological Interest, Fifth Edition, NIH Publication 91-3242, Bethesda Md. (1991), vols. 1-3.

In certain embodiments, the antigen-binding domain of an antibody is formed from two variable (V) regions of about 110 amino acids, one each from the light (VL) and heavy (VH) chains, that both present three hypervariable loops (HVRs) or complementarity-determining regions (CDRs). Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described in Winter et al., Ann. Rev. Immunol., 12: 433-455 (1994). As used herein, scFv encoding phage clones and Fab encoding phage clones are collectively referred to as “Fv phage clones” or “Fv clones.”

Repertoires of VH and VL genes can be separately cloned by polymerase chain reaction (PCR) and recombined randomly in phage libraries, which can then be searched for antigen-binding clones as described in Winter et al., Ann. Rev. Immunol., 12: 433-455 (1994). Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described by Hoogenboom and Winter, J. Mol. Biol., 227: 381-388 (1992).

In certain embodiments, filamentous phage is used to display antibody fragments by fusion to the minor coat protein pIII. The antibody fragments can be displayed as single chain Fv fragments, in which VH and VL domains are connected on the same polypeptide chain by a flexible polypeptide spacer, e.g. as described by Marks et al., J. Mol. Biol., 222: 581-597 (1991), or as Fab fragments, in which one chain is fused to pIII and the other is secreted into the bacterial host cell periplasm where assembly of a Fab-coat protein structure which becomes displayed on the phage surface by displacing some of the wild type coat proteins, e.g. as described in Hoogenboom et al., Nucl. Acids Res., 19: 4133-4137 (1991).

In general, nucleic acids encoding antibody gene fragments are obtained from immune cells harvested from humans or animals. If a library biased in favor of anti-antigen clones is desired, the subject is immunized with antigen to generate an antibody response, and spleen cells and/or circulating B cells other peripheral blood lymphocytes (PBLs) are recovered for library construction. In one embodiment, a human antibody gene fragment library biased in favor of anti-antigen clones is obtained by generating an anti-antigen antibody response in transgenic mice carrying a functional human immunoglobulin gene array (and lacking a functional endogenous antibody production system) such that antigen immunization gives rise to B cells producing human antibodies against antigen. The generation of human antibody-producing transgenic mice is described below.

Additional enrichment for anti-antigen reactive cell populations can be obtained by using a suitable screening procedure to isolate B cells expressing antigen-specific membrane bound antibody, e.g., by cell separation using antigen affinity chromatography or adsorption of cells to fluorochrome-labeled antigen followed by flow-activated cell sorting (FACS).

Alternatively, the use of spleen cells and/or B cells or other PBLs from an unimmunized donor provides a better representation of the possible antibody repertoire, and also permits the construction of an antibody library using any animal (human or non-human) species in which antigen is not antigenic. For libraries incorporating in vitro antibody gene construction, stem cells are harvested from the subject to provide nucleic acids encoding unrearranged antibody gene segments. The immune cells of interest can be obtained from a variety of animal species, such as human, mouse, rat, lagomorpha, luprine, canine, feline, porcine, bovine, equine, and avian species, etc.

Nucleic acid encoding antibody variable gene segments (including VH and VL segments) are recovered from the cells of interest and amplified. In the case of rearranged VH and VL gene libraries, the desired DNA can be obtained by isolating genomic DNA or mRNA from lymphocytes followed by polymerase chain reaction (PCR) with primers matching the 5′ and 3′ ends of rearranged VH and VL genes as described in Orlandi et al., Proc. Natl. Acad. Sci. (USA), 86: 3833-3837 (1989), thereby making diverse V gene repertoires for expression. The V genes can be amplified from cDNA and genomic DNA, with back primers at the 5′ end of the exon encoding the mature V-domain and forward primers based within the J-segment as described in Orlandi et al. (1989) and in Ward et al., Nature, 341: 544-546 (1989). However, for amplifying from cDNA, back primers can also be based in the leader exon as described in Jones et al., Biotechnol., 9: 88-89 (1991), and forward primers within the constant region as described in Sastry et al., Proc. Natl. Acad. Sci. (USA), 86: 5728-5732 (1989). To maximize complementarity, degeneracy can be incorporated in the primers as described in Orlandi et al. (1989) or Sastry et al. (1989). In certain embodiments, library diversity is maximized by using PCR primers targeted to each V-gene family in order to amplify all available VH and VL arrangements present in the immune cell nucleic acid sample, e.g. as described in the method of Marks et al., J. Mol. Biol., 222: 581-597 (1991) or as described in the method of Orum et al., Nucleic Acids Res., 21: 4491-4498 (1993). For cloning of the amplified DNA into expression vectors, rare restriction sites can be introduced within the PCR primer as a tag at one end as described in Orlandi et al. (1989), or by further PCR amplification with a tagged primer as described in Clackson et al., Nature, 352: 624-628 (1991).

Repertoires of synthetically rearranged V genes can be derived in vitro from V gene segments. Most of the human VH-gene segments have been cloned and sequenced (reported in Tomlinson et al., J. Mol. Biol., 227: 776-798 (1992)), and mapped (reported in Matsuda et al., Nature Genet., 3: 88-94 (1993); these cloned segments (including all the major conformations of the H1 and H2 loop) can be used to generate diverse VH gene repertoires with PCR primers encoding H3 loops of diverse sequence and length as described in Hoogenboom and Winter, J. Mol. Biol., 227: 381-388 (1992). VH repertoires can also be made with all the sequence diversity focused in a long H3 loop of a single length as described in Barbas et al., Proc. Natl. Acad. Sci. USA, 89: 4457-4461 (1992). Human Vκ and Vλ segments have been cloned and sequenced (reported in Williams and Winter, Eur. J. Immunol., 23: 1456-1461 (1993)) and can be used to make synthetic light chain repertoires. Synthetic V gene repertoires, based on a range of VH and VL folds, and L3 and H3 lengths, will encode antibodies of considerable structural diversity. Following amplification of V-gene encoding DNAs, germline V-gene segments can be rearranged in vitro according to the methods of Hoogenboom and Winter, J. Mol. Biol., 227: 381-388 (1992).

Repertoires of antibody fragments can be constructed by combining VH and VL gene repertoires together in several ways. Each repertoire can be created in different vectors, and the vectors recombined in vitro, e.g., as described in Hogrefe et al., Gene, 128: 119-126 (1993), or in vivo by combinatorial infection, e.g., the loxP system described in Waterhouse et al., Nucl. Acids Res., 21: 2265-2266 (1993). The in vivo recombination approach exploits the two-chain nature of Fab fragments to overcome the limit on library size imposed by E. coli transformation efficiency. Naive VH and VL repertoires are cloned separately, one into a phagemid and the other into a phage vector. The two libraries are then combined by phage infection of phagemid-containing bacteria so that each cell contains a different combination and the library size is limited only by the number of cells present (about 10¹² clones). Both vectors contain in vivo recombination signals so that the VH and VL genes are recombined onto a single replicon and are co-packaged into phage virions. These huge libraries provide large numbers of diverse antibodies of good affinity (K_{d}⁻¹ of about 10⁻⁸ M).

Alternatively, the repertoires may be cloned sequentially into the same vector, e.g. as described in Barbas et al., Proc. Natl. Acad. Sci. USA, 88: 7978-7982 (1991), or assembled together by PCR and then cloned, e.g. as described in Clackson et al., Nature, 352: 624-628 (1991). PCR assembly can also be used to join VH and VL DNAs with DNA encoding a flexible peptide spacer to form single chain Fv (scFv) repertoires. In yet another technique, “in cell PCR assembly” is used to combine VH and VL genes within lymphocytes by PCR and then clone repertoires of linked genes as described in Embleton et al., Nucl. Acids Res., 20: 3831-3837 (1992).

The antibodies produced by naive libraries (either natural or synthetic) can be of moderate affinity (K_{d}⁻¹ of about 10⁶ to 10⁷ M⁻¹), but affinity maturation can also be mimicked in vitro by constructing and reselecting from secondary libraries as described in Winter et al. (1994), supra. For example, mutation can be introduced at random in vitro by using error-prone polymerase (reported in Leung et al., Technique 1: 11-15 (1989)) in the method of Hawkins et al., J. Mol. Biol., 226: 889-896 (1992) or in the method of Gram et al., Proc. Natl. Acad. Sci USA, 89: 3576-3580 (1992). Additionally, affinity maturation can be performed by randomly mutating one or more CDRs, e.g. using PCR with primers carrying random sequence spanning the CDR of interest, in selected individual Fv clones and screening for higher affinity clones. WO 9607754 (published 14 Mar. 1996) described a method for inducing mutagenesis in a complementarity determining region of an immunoglobulin light chain to create a library of light chain genes. Another effective approach is to recombine the VH or VL domains selected by phage display with repertoires of naturally occurring V domain variants obtained from unimmunized donors and screen for higher affinity in several rounds of chain reshuffling as described in Marks et al., Biotechnol., 10: 779-783 (1992). This technique allows the production of antibodies and antibody fragments with affinities of about 10⁻⁹ M or less.

Screening of the libraries can be accomplished by various techniques known in the art. For example, antigen can be used to coat the wells of adsorption plates, expressed on host cells affixed to adsorption plates or used in cell sorting, or conjugated to biotin for capture with streptavidin-coated beads, or used in any other method for panning phage display libraries.

The phage library samples are contacted with immobilized antigen under conditions suitable for binding at least a portion of the phage particles with the adsorbent. Normally, the conditions, including pH, ionic strength, temperature and the like are selected to mimic physiological conditions. The phages bound to the solid phase are washed and then eluted by acid, e.g. as described in Barbas et al., Proc. Natl. Acad. Sci USA, 88: 7978-7982 (1991), or by alkali, e.g. as described in Marks et al., J. Mol. Biol., 222: 581-597 (1991), or by antigen competition, e.g. in a procedure similar to the antigen competition method of Clackson et al., Nature, 352: 624-628 (1991). Phages can be enriched 20-1,000-fold in a single round of selection. Moreover, the enriched phages can be grown in bacterial culture and subjected to further rounds of selection.

The efficiency of selection depends on many factors, including the kinetics of dissociation during washing, and whether multiple antibody fragments on a single phage can simultaneously engage with antigen. Antibodies with fast dissociation kinetics (and weak binding affinities) can be retained by use of short washes, multivalent phage display and high coating density of antigen in solid phase. The high density not only stabilizes the phage through multivalent interactions, but favors rebinding of phage that has dissociated. The selection of antibodies with slow dissociation kinetics (and good binding affinities) can be promoted by use of long washes and monovalent phage display as described in Bass et al., Proteins, 8: 309-314 (1990) and in WO 92/09690, and a low coating density of antigen as described in Marks et al., Biotechnol., 10: 779-783 (1992).

It is possible to select between phage antibodies of different affinities, even with affinities that differ slightly, for antigen. However, random mutation of a selected antibody (e.g. as performed in some affinity maturation techniques) is likely to give rise to many mutants, most binding to antigen, and a few with higher affinity. With limiting antigen, rare high affinity phage could be competed out. To retain all higher affinity mutants, phages can be incubated with excess biotinylated antigen, but with the biotinylated antigen at a concentration of lower molarity than the target molar affinity constant for antigen. The high affinity-binding phages can then be captured by streptavidin-coated paramagnetic beads. Such “equilibrium capture” allows the antibodies to be selected according to their affinities of binding, with sensitivity that permits isolation of mutant clones with as little as two-fold higher affinity from a great excess of phages with lower affinity. Conditions used in washing phages bound to a solid phase can also be manipulated to discriminate on the basis of dissociation kinetics.

Anti-antigen clones may be selected based on activity. In certain embodiments, the invention provides anti-antigen antibodies that bind to living cells that naturally express antigen or bind to free floating antigen or antigen attached to other cellular structures. Fv clones corresponding to such anti-antigen antibodies can be selected by (1) isolating anti-antigen clones from a phage library as described above, and optionally amplifying the isolated population of phage clones by growing up the population in a suitable bacterial host; (2) selecting antigen and a second protein against which blocking and non-blocking activity, respectively, is desired; (3) adsorbing the anti-antigen phage clones to immobilized antigen; (4) using an excess of the second protein to elute any undesired clones that recognize antigen-binding determinants which overlap or are shared with the binding determinants of the second protein; and (5) eluting the clones which remain adsorbed following step (4). Optionally, clones with the desired blocking/non-blocking properties can be further enriched by repeating the selection procedures described herein one or more times.

DNA encoding hybridoma-derived monoclonal antibodies or phage display Fv clones is readily isolated and sequenced using conventional procedures (e.g. by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells. Review articles on recombinant expression in bacteria of antibody-encoding DNA include Skerra et al., Curr. Opinion in Immunol., 5: 256 (1993) and Pluckthun, Immunol. Revs, 130: 151 (1992).

DNA encoding the Fv clones can be combined with known DNA sequences encoding heavy chain and/or light chain constant regions (e.g. the appropriate DNA sequences can be obtained from Kabat et al., supra) to form clones encoding full or partial length heavy and/or light chains. It will be appreciated that constant regions of any isotype can be used for this purpose, including IgG, IgM, IgA, IgD, and IgE constant regions, and that such constant regions can be obtained from any human or animal species. An Fv clone derived from the variable domain DNA of one animal (such as human) species and then fused to constant region DNA of another animal species to form coding sequence(s) for “hybrid,” full length heavy chain and/or light chain is included in the definition of “chimeric” and “hybrid” antibody as used herein. In certain embodiments, an Fv clone derived from human variable DNA is fused to human constant region DNA to form coding sequence(s) for full- or partial-length human heavy and/or light chains.

DNA encoding anti-antigen antibody derived from a hybridoma can also be modified, for example, by substituting the coding sequence for human heavy- and light-chain constant domains in place of homologous murine sequences derived from the hybridoma clone (e.g. as in the method of Morrison et al., Proc. Natl. Acad. Sci. USA, 81: 6851-6855 (1984)). DNA encoding a hybridoma- or Fv clone-derived antibody or fragment can be further modified by covalently joining to the immunoglobulin coding sequence all or part of the coding sequence for a non-immunoglobulin polypeptide. In this manner, “chimeric” or “hybrid” antibodies are prepared that have the binding specificity of the Fv clone or hybridoma clone-derived antibodies.

(iv) Humanized and Human Antibodies

Various methods for humanizing non-human antibodies are known in the art. For example, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad Sci. USA, 89:4285 (1992); Presta et al., J. Immunol., 151:2623 (1993)).

It is further important that antibodies be humanized with retention of high affinity for the antigen and other favorable biological properties. To achieve this goal, according to one embodiment of the method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human antibodies in the formulations and compositions described herein can be constructed by combining Fv clone variable domain sequence(s) selected from human-derived phage display libraries with known human constant domain sequence(s) as described above. Alternatively, human monoclonal antibodies can be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).

It is possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al, Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992).

Gene shuffling can also be used to derive human antibodies from non-human, e.g. rodent, antibodies, where the human antibody has similar affinities and specificities to the starting non-human antibody. According to this method, which is also called “epitope imprinting”, either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e. the epitope governs (imprints) the choice of the human chain partner. When the process is repeated in order to replace the remaining non-human chain, a human antibody is obtained (see PCT WO 93/06213 published Apr. 1, 1993). Unlike traditional humanization of non-human antibodies by CDR grafting, this technique provides completely human antibodies, which have no FR or CDR residues of non-human origin.

(v) Antibody Fragments

Antibody fragments may be generated by traditional means, such as enzymatic digestion, or by recombinant techniques. In certain circumstances there are advantages of using antibody fragments, rather than whole antibodies. The smaller size of the fragments allows for rapid clearance, and may lead to improved access to solid tumors. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific.

(vi) Multispecific Antibodies

Multispecific antibodies have binding specificities for at least two different epitopes, where the epitopes are usually from different antigens. While such molecules normally will only bind two different epitopes (i.e. bispecific antibodies, BsAbs), antibodies with additional specificities such as trispecific antibodies are encompassed by this expression when used herein. Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is typical to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in WO96/27011, the interface between a pair of antibody molecules can be engineered to maximize the percentage of heterodimers which are recovered from recombinant cell culture. One interface comprises at least a part of the C_{H} 3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al, J. Immunol, 152:5368 (1994).

Antibodies with more than two valencies are contemplated. For example, trispecific antibodies can be prepared. Tuft et al. J. Immunol. 147: 60 (1991).

(vii) Single-Domain Antibodies

In some embodiments, an antibody described herein is a single-domain antibody. A single-domain antibody is a single polypeptide chain comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1). In one embodiment, a single-domain antibody consists of all or a portion of the heavy chain variable domain of an antibody.

(viii) Antibody Variants

In some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

(ix) Antibody Derivatives

The antibodies in the formulations and compositions of the invention can be further modified to contain additional nonproteinaceous moieties that are known in the art and readily available. In certain embodiments, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

(x) Vectors, Host Cells, and Recombinant Methods

Antibodies may also be produced using recombinant methods. For recombinant production of an anti-antigen antibody, nucleic acid encoding the antibody is isolated and inserted into a replicable vector for further cloning (amplification of the DNA) or for expression. DNA encoding the antibody may be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

(a) Signal Sequence Component

An antibody in the formulations and compositions described herein may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (e.g., cleaved by a signal peptidase) by the host cell. For prokaryotic host cells that do not recognize and process a native antibody signal sequence, the signal sequence is substituted by a prokaryotic signal sequence selected, for example, from the group of the alkaline phosphatase, penicillinase, lpp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

(b) Origin of Replication

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the origin of replication from the 2μ plasmid is suitable for yeast, and various viral origins of replication (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

(c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up antibody-encoding nucleic acid, such as DHFR, glutamine synthetase (GS), thymidine kinase, metallothionein-I and -II, preferably primate metallothionein genes, adenosine deaminase, ornithine decarboxylase, etc.

For example, cells transformed with the DHFR gene are identified by culturing the transformants in a culture medium containing methotrexate (Mtx), a competitive antagonist of DHFR. Under these conditions, the DHFR gene is amplified along with any other co-transformed nucleic acid. A Chinese hamster ovary (CHO) cell line deficient in endogenous DHFR activity (e.g., ATCC CRL-9096) may be used.

Alternatively, cells transformed with the GS gene are identified by culturing the transformants in a culture medium containing L-methionine sulfoximine (Msx), an inhibitor of GS. Under these conditions, the GS gene is amplified along with any other co-transformed nucleic acid. The GS selection/amplification system may be used in combination with the DHFR selection/amplification system described above.

Alternatively, host cells (particularly wild-type hosts that contain endogenous DHFR) transformed or co-transformed with DNA sequences encoding an antibody of interest, wild-type DHFR gene, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

(d) Promoter Component

Expression and cloning vectors generally contain a promoter that is recognized by the host organism and is operably linked to nucleic acid encoding an antibody. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding an antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoter sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Antibody transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), or from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

(e) Enhancer Element Component

Transcription of a DNA encoding an antibody by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody-encoding sequence, but is preferably located at a site 5′ from the promoter.

(f) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding antibody. One useful transcription termination component is the bovine growth hormone polyadenylation region. See WO94/11026 and the expression vector disclosed therein.

(g) Selection and Transformation of Host Cells

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Full length antibody, antibody fusion proteins, and antibody fragments can be produced in bacteria, in particular when glycosylation and Fc effector function are not needed, such as when the therapeutic antibody is conjugated to a cytotoxic agent (e.g., a toxin) that by itself shows effectiveness in tumor cell destruction. Full length antibodies have greater half-life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), U.S. Pat. No. 5,840,523 (Simmons et al.), which describes translation initiation region (TIR) and signal sequences for optimizing expression and secretion. See also Charlton, Methods in Molecular Biology, Vol. 248 (B. K. C. Lo, ed., Humana Press, Totowa, N.J., 2003), pp. 245-254, describing expression of antibody fragments in E. coli. After expression, the antibody may be isolated from the E. coli cell paste in a soluble fraction and can be purified through, e.g., a protein A or G column depending on the isotype. Final purification can be carried out similar to the process for purifying antibody expressed e.g., in CHO cells.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger. For a review discussing the use of yeasts and filamentous fungi for the production of therapeutic proteins, see, e.g., Gerngross, Nat. Biotech. 22:1409-1414 (2004).

Certain fungi and yeast strains may be selected in which glycosylation pathways have been “humanized,” resulting in the production of an antibody with a partially or fully human glycosylation pattern. See, e.g., Li et al., Nat. Biotech. 24:210-215 (2006) (describing humanization of the glycosylation pathway in Pichia pastoris); and Gerngross et al., supra.

Suitable host cells for the expression of glycosylated antibody are also derived from multicellular organisms (invertebrates and vertebrates). Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the invention, particularly for transfection of Spodoptera frugiperda cells.

Plant cell cultures of cotton, corn, potato, soybean, petunia, tomato, duckweed (Leninaceae), alfalfa (M. truncatula), and tobacco can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may be used as hosts, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J., 2003), pp. 255-268.

Host cells are transformed with the above-described expression or cloning vectors for antibody production and cultured in conventional nutrient media modified as appropriate for inducing promoters, selecting transformants, or amplifying the genes encoding the desired sequences.

(h) Culturing the Host Cells

The host cells used to produce an antibody may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

(xi) Purification of Antibody

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, hydrophobic interaction chromatography, gel electrophoresis, dialysis, and affinity chromatography, with affinity chromatography being among one of the typically preferred purification steps. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). Protein L can be used to purify antibodies based on the kappa light chain (Nilson et al., J. Immunol. Meth. 164(1):33-40, 1993). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

In general, various methodologies for preparing antibodies for use in research, testing, and clinical are well-established in the art, consistent with the above-described methodologies and/or as deemed appropriate by one skilled in the art for a particular antibody of interest.

B. Selecting Biologically Active Antibodies

Antibodies produced as described above may be subjected to one or more “biological activity” assays to select an antibody with beneficial properties from a therapeutic perspective. The antibody may be screened for its ability to bind the antigen against which it was raised. For example, for an anti-DR5 antibody (e.g., drozitumab), the antigen binding properties of the antibody can be evaluated in an assay that detects the ability to bind to a death receptor 5 (DR5).

In another embodiment, the affinity of the antibody may be determined by saturation binding; ELISA; and/or competition assays (e.g. RIA's), for example.

Also, the antibody may be subjected to other biological activity assays, e.g., in order to evaluate its effectiveness as a therapeutic. Such assays are known in the art and depend on the target antigen and intended use for the antibody.

To screen for antibodies which bind to a particular epitope on the antigen of interest, a routine cross-blocking assay such as that described in Antibodies, A Laboratory Manual, Cold Spring Harbor Laboratory, Ed Harlow and David Lane (1988), can be performed. Alternatively, epitope mapping, e.g. as described in Champe et al., J. Biol. Chem. 270:1388-1394 (1995), can be performed to determine whether the antibody binds an epitope of interest.

C. Preparation of the Formulations

Provided herein are methods of preparing a liquid formulation comprising a protein and NAT which prevents oxidation of the protein in the liquid formulation. The liquid formulation may be prepared by mixing the protein having the desired degree of purity with NAT which prevents oxidation of the protein in the liquid formulation. In certain embodiments, the protein to be formulated has not been subjected to prior lyophilization and the formulation of interest herein is an aqueous formulation. In some embodiments, the protein is a therapeutic protein. In some embodiments, the protein is an antibody. In further embodiments, the antibody is a polyclonal antibody, a monoclonal antibody, a humanized antibody, a human antibody, a chimeric antibody, a multispecific antibody, or an antibody fragment. In certain embodiments, the antibody is a full length antibody. In one embodiment, the antibody in the formulation is an antibody fragment, such as an F(ab′)₂, in which case problems that may not occur for the full length antibody (such as clipping of the antibody to Fab) may need to be addressed. The therapeutically effective amount of protein present in the formulation is determined by taking into account the desired dose volumes and mode(s) of administration, for example. Exemplary protein concentrations in the formulation include from about 1 mg/mL to more than about 250 mg/mL, from about 1 mg/mL to about 250 mg/mL, from about 10 mg/mL to about 250 mg/mL, from about 15 mg/mL to about 225 mg/mL, from about 20 mg/mL to about 200 mg/mL, from about 25 mg/mL to about 175 mg/mL, from about 25 mg/mL to about 150 mg/mL, from about 25 mg/mL to about 100 mg/mL, from about 30 mg/mL to about 100 mg/mL or from about 45 mg/mL to about 55 mg/mL. In some embodiments, the protein described herein is susceptible to oxidation. In some embodiments, one or more of the amino acids selected from the group consisting of methionine, cysteine, histidine, tryptophan, and tyrosine in the protein is susceptible to oxidation. In some embodiments, tryptophan in the protein is susceptible to oxidation. In some embodiments, the protein comprises a tryptophan residue with a solvent-accessible surface area (SASA) greater than about 50 Å² to about 250 Å² (such as greater than about any of 50, 60, 70, 80, 90, 100, 120, 140, 160, 180, 200, 225, or 250 Å², including any ranges between these values). In some embodiments, the SASA is greater than about 80 Å². In some embodiments, the protein comprises a tryptophan residue with a SASA greater than about 15% to about 45% (such as greater than about any of 15, 20, 25, 30, 35, 40, or 45%). In some embodiments, the SASA is greater than about 30%. In some embodiments, SASA of a tryptophan residue is measured at a pH range from about 4.0 to about 8.5. In some embodiments, SASA of a tryptophan residue is measured at a temperature ranging from about 5° C. to about 40° C. In some embodiments, SASA of a tryptophan residue is measured at a salt concentration ranging from about 0 mM to about 500 mM. In some embodiments, SASA of a tryptophan residue is measured at a pH of about 5.0 to about 7.5, a temperature of about 5° C. to about 25° C. and a salt concentration of about 0 mM to about 500 mM. In some embodiments, the protein comprises at least one tryptophan residue predicted to be susceptible to oxidation by a machine learning algorithm trained on associations of tryptophan residue oxidation susceptibility with a plurality of molecule descriptors of the tryptophan residue based on MD simulations. In some embodiments, an antibody provided herein is susceptible to oxidation in the Fab portion and/or the Fc portion of the antibody. In some embodiments, an antibody provided herein is susceptible to oxidation at a tryptophan amino acid in the Fab portion of the antibody. In a further embodiment, the tryptophan amino acid susceptible to oxidation is in a CDR of the antibody. In some embodiments, an antibody provided herein is susceptible to oxidation at a methionine amino acid in the Fc portion of the antibody. In some embodiments, the liquid formulation further comprises at least one additional protein according to any of the proteins described herein.

The liquid formulations provided by the invention herein comprise a protein and NAT which prevents oxidation of the protein in the liquid formulation. In some embodiments, the NAT in the formulation is at a concentration from about 0.1 mM to more than about 10 mM, or up to the highest concentration that the NAT is soluble to in the formulation. In certain embodiments, the NAT in the formulation is at a concentration from about 0.1 mM to about 10 mM, about 0.1 mM to about 9 mM, from about 0.1 mM to about 8 mM, from about 0.1 mM to about 7 mM, from about 0.1 mM to about 6 mM, from about 0.1 mM to about 5 mM, from about 0.1 mM to about 4 mM, from about 0.1 mM to about 3 mM, from about 0.1 mM to about 2 mM, from about 0.3 mM to about 2 mM, from about 0.5 mM to about 2 mM, from about 0.6 mM to about 1.5 mM, or from about 0.8 mM to about 1.25 mM. In some embodiments, the NAT in the formulation is about 1 mM. In some embodiments, the NAT prevents oxidation of one or more amino acids in the protein. In some embodiments, the NAT prevents oxidation of one or more amino acids in the protein selected from group consisting of tryptophan, methionine, tyrosine, histidine, and/or cysteine. In some embodiments, the NAT prevents oxidation of tryptophan in the protein. In some embodiments, the NAT prevents oxidation of the protein by a reactive oxygen species (ROS). In a further embodiment, the reactive oxygen species is selected from the group consisting of a singlet oxygen, a superoxide (O₂—), an alkoxyl radical, a peroxyl radical, a hydrogen peroxide (H₂O₂), a dihydrogen trioxide (H₂O₃), a hydrotrioxy radical (HO₃.), ozone (O₃), a hydroxyl radical, and an alkyl peroxide. In a further embodiment, the NAT prevents oxidation of one or more amino acids in the Fab portion of an antibody. In another further embodiment, the NAT prevents oxidation of one or more amino acids in the Fc portion of an antibody.

In some embodiments, liquid formulations provided by the invention herein comprise a protein and NAT which prevents oxidation of the protein in the liquid formulation, wherein the oxidation of the protein is reduced by about 40% to about 100%. In some embodiments, the oxidation of the protein is reduced by about any of 40%, 45%, 50%, 55%, 60%, 65%, 70%, 75%, 80%, 85%, 90%, 95%, 96%, 97%, 98%, 99%, or 100%, including any ranges between these values.

The amount of oxidation in a protein can be determined, for example, using one or more of RP-HPLC, LC/MS, or tryptic peptide mapping. In some embodiments, the oxidation in a protein is determined as a percentage using one or more of RP-HPLC, LC/MS, or tryptic peptide mapping and the formula of:

$$\% Oxida tion = 100 \times \frac{Oxidized Peak Area}{Peak Area + Oxidized Peak Area}$$

In some embodiments, liquid formulations provided by the invention herein comprise a protein and NAT which prevents oxidation of the protein in the liquid formulation, wherein no more than about 40% to about 0% of the protein is oxidized. In some embodiments, no more than about any of 40%, 35%, 30%, 25%, 20%, 15%, 10%, 5%, 4%, 3%, 2%, 1%, or 0%, including any ranges between these values, of the protein is oxidized.

In some embodiments, liquid formulations provided by the invention herein comprise a protein and NAT which prevents oxidation of the protein in the liquid formulation, wherein the oxidation of at least one oxidation labile tryptophan in the protein is reduced by about 40% to about 100%. In some embodiments, the oxidation of the oxidation labile tryptophan is reduced by about any of 40%, 45%, 50%, 55%, 60%, 65%, 70%, 75%, 80%, 85%, 90%, 95%, 96%, 97%, 98%, 99%, or 100%, including any ranges between these values. In some embodiments, the oxidation of each of the oxidation labile tryptophan residues in the protein is reduced by about 40% to about 100% (such as about any of 40%, 45%, 50%, 55%, 60%, 65%, 70%, 75%, 80%, 85%, 90%, 95%, 96%, 97%, 98%, 99%, or 100%, including any ranges between these values).

In some embodiments, liquid formulations provided by the invention herein comprise a protein and NAT which prevents oxidation of the protein in the liquid formulation, wherein no more than about 40% to about 0% of at least one oxidation labile tryptophan in the protein is oxidized. In some embodiments, no more than about any of 40%, 35%, 30%, 25%, 20%, 15%, 10%, 5%, 4%, 3%, 2%, 1%, or 0%, including any ranges between these values, of the oxidation labile tryptophan is oxidized. In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of each of the oxidation labile tryptophan residues in the protein is oxidized.

In some embodiments, the liquid formulation further comprises one or more excipients selected from the group consisting of a stabilizer, a buffer, a surfactant, and a tonicity agent. A liquid formulation of the invention is prepared in a pH-buffered solution. The buffer of this invention has a pH in the range from about 4.0 to about 9.0. In certain embodiments the pH is in the range from pH 4.0 to 8.5, in the range from pH 4.0 to 8.0, in the range from pH 4.0 to 7.5, in the range from pH 4.0 to 7.0, in the range from pH 4.0 to 6.5, in the range from pH 4.0 to 6.0, in the range from pH 4.0 to 5.5, in the range from pH 4.0 to 5.0, in the range from pH 4.0 to 4.5, in the range from pH 4.5 to 9.0, in the range from pH 5.0 to 9.0, in the range from pH 5.5 to 9.0, in the range from pH 6.0 to 9.0, in the range from pH 6.5 to 9.0, in the range from pH 7.0 to 9.0, in the range from pH 7.5 to 9.0, in the range from pH 8.0 to 9.0, in the range from pH 8.5 to 9.0, in the range from pH 5.7 to 6.8, in the range from pH 5.8 to 6.5, in the range from pH 5.9 to 6.5, in the range from pH 6.0 to 6.5, or in the range from pH 6.2 to 6.5. In certain embodiments of the invention, the liquid formulation has a pH of 6.2 or about 6.2. In certain embodiments of the invention, the liquid formulation has a pH of 6.0 or about 6.0. Examples of buffers that will control the pH within this range include organic and inorganic acids and salts thereof. For example, acetate (e.g., histidine acetate, arginine acetate, sodium acetate), succinate (e.g., histidine succinate, arginine succinate, sodium succinate), gluconate, phosphate, fumarate, oxalate, lactate, citrate, and combinations thereof. The buffer concentration can be from about 1 mM to about 600 mM, depending, for example, on the buffer and the desired isotonicity of the formulation. In certain embodiments, the formulation comprises a histidine buffer (e.g., in the concentration from about 5 mM to 100 mM). Examples of histidine buffers include histidine chloride, histidine acetate, histidine phosphate, histidine sulfate, histidine succinate, etc. In certain embodiments, the formulation comprises histidine and arginine (e.g., histidine chloride-arginine chloride, histidine acetate-arginine acetate, histidine phosphate-arginine phosphate, histidine sulfate-arginine sulfate, histidine succinate-arginine succinate, etc.). In certain embodiments, the formulation comprises histidine in the concentration from about 5 mM to 100 mM and the arginine is in the concentration of 50 mM to 500 mM. In one embodiment, the formulation comprises histidine acetate (e.g., about 20 mM)-arginine acetate (e.g., about 150 mM). In certain embodiments, the formulation comprises histidine succinate (e.g., about 20 mM)-arginine succinate (e.g., about 150 mM). In certain embodiments, histidine in the formulation front about 10 mM to about, 35 mM, about 10 mM to about 30 mM, about 10 mM to about 25 mM, about 10 mM to about 20 mM, about 10 mM to about 15 mM, about 15 mM to about 35 mM, about 20 mM to about 35 mM, about 20 mM to about 30 mM or about 20 mM to about 25 mM. In further embodiments, the arginine in the formulation is from about 50 mM to about 500 mM (e.g., about 100 mM, about 150 mM, or about 200 mM).

The liquid formulation of the invention can further comprise a saccharide, such as a disaccharide (e.g., trehalose or sucrose). A “saccharide” as used herein includes the general composition (CH₂O)n and derivatives thereof, including monosaccharides, disaccharides, trisaccharides, polysaccharides, sugar alcohols, reducing sugars, nonreducing sugars, etc. Examples of saccharides herein include glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, etc.

A surfactant can optionally be added to the liquid formulation. Exemplary surfactants include nonionic surfactants such as polysorbates (e.g. polysorbates 20, 80, etc.) or poloxamers (e.g. poloxamer 188, etc.). The amount of surfactant added is such that it reduces aggregation of the formulated antibody and/or minimizes the formation of particulates in the formulation and/or reduces adsorption. For example, the surfactant may be present in the formulation in an amount from about 0.001% to more than about 1.0%, weight/volume. In some embodiments, the surfactant is present in the formulation in an amount from about 0.001% to about 1.0%, from about 0.001% to about 0.5%, from about 0.005% to about 0.2%, from about 0.01% to about 0.1%, from about 0.02% to about 0.06%, or about 0.03% to about 0.05%, weight/volume. In certain embodiments, the surfactant is present in the formulation in an amount of 0.04% or about 0.04%, weight/volume. In certain embodiments, the surfactant is present in the formulation in an amount of 0.02% or about 0.02%, weight/volume. In one embodiment, the formulation does not comprise a surfactant.

In one embodiment, the formulation contains the above-identified agents (e.g., antibody, buffer, saccharide, and/or surfactant) and is essentially free of one or more preservatives, such as benzyl alcohol, phenol, m-cresol, chlorobutanol and benzethonium Cl. In another embodiment, a preservative may be included in the formulation, particularly where the formulation is a multidose formulation. The concentration of preservative may be in the range from about 0.1% to about 2%, preferably from about 0.5% to about 1%. One or more other pharmaceutically acceptable carriers, excipients or stabilizers such as those described in Remington's Pharmaceutical Sciences 16th edition, Osol, A. Ed. (1980) may be included in the formulation provided that they do not adversely affect the desired characteristics of the formulation. Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The formulation may further comprise metal ion chelators. Metal ion chelators are well known by those of skill in the art and include, but are not necessarily limited to aminopolycarboxylates, EDTA (ethylenediaminetetraacetic acid), EGTA (ethylene glycol-bis(beta-aminoethyl ether)-N,N,N′,N′-tetraacetic acid), NTA (nitrilotriacetic acid), EDDS (ethylene diamine disuccinate), PDTA (1,3-propylenediaminetetraacetic acid), DTPA (diethylenetriaminepentaacetic acid), ADA (beta-alaninediacetic acid), MGCA (methylglycinediacetic acid), etc. Additionally, some embodiments herein comprise phosphonates/phosphonic acid chelators.

Tonicity agents are present to adjust or maintain the tonicity of liquid in a composition. When used with large, charged biomolecules such as proteins and antibodies, they may also serve as “stabilizers” because they can interact with the charged groups of the amino acid side chains, thereby lessening the potential for inter- and intra-molecular interactions. Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

The formulation herein may also contain more than one protein or a small molecule drug as necessary for the particular indication being treated, preferably those with complementary activities that do not adversely affect the other protein. For example, where the antibody is anti-DR5 (e.g., drozitumab), it may be combined with another agent (e.g., a chemotherapeutic agent, and anti-neoplastic agent).

In some embodiments, the formulation is for in vivo administration. In some embodiments, the formulation is sterile. The formulation may be rendered sterile by filtration through sterile filtration membranes. The therapeutic formulations herein generally are placed into a container having a sterile access port, for example, an intravenous solution bag or vial having a stopper pierceable by a hypodermic injection needle. The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional, intraarticular, or intravitreal routes, topical administration, inhalation or by sustained release or extended-release means.

The liquid formulation of the invention may be stable upon storage. In some embodiments, the protein in the liquid formulation is stable upon storage at about 0 to about 5° C. (such as about any of 1, 2, 3, or 4° C.) for at least about 12 months (such as at least about any of 15, 18, 21, 24, 27, 30, 33, 36 months, or greater). In some embodiments, the physical stability, chemical stability, or biological activity of the protein in the liquid formulation is evaluated or measured. Any methods known the art may be used to evaluate the stability and biological activity. In some embodiments, the stability is measured by oxidation of the protein in the liquid formulation after storage. Stability can be tested by evaluating physical stability, chemical stability, and/or biological activity of the antibody in the formulation around the time of formulation as well as following storage. Physical and/or stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may result in aggregation, deamidation (e.g. Asn deamidation), oxidation (e.g. Trp oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc. In some embodiments, the oxidation in a protein is determined using one or more of RP-HPLC, LC/MS, or tryptic peptide mapping. In some embodiments, the oxidation in an antibody is determined as a percentage using one or more of RP-HPLC, LC/MS, or tryptic peptide mapping and the formula of:

$$\% Fab Oxida tion = 100 \times \frac{Oxidized Fab Peak Area}{Fab Peak Area + Oxidized Fab Peak Area}$$

$$\% Fc Oxida tion = 100 \times \frac{Oxidized Fc Peak Area}{Fc Peak Area + Oxidized Fc Peak Area}$$

The formulations to be used for in vivo administration should be sterile. This is readily accomplished by filtration through sterile filtration membranes, prior to, or following, preparation of the formulation.

Also provided herein are methods of making a liquid formulation or preventing oxidation of a protein in a liquid formulation comprising adding an amount of NAT that prevents oxidation of a protein to a liquid formulation. In certain embodiments, the liquid formulation comprises an antibody. The amount of the NAT that prevents oxidation of the protein as provided herein is from about 0.1 mM to about 10 mM or any of the amounts disclosed herein. In some embodiments, the liquid formulation further comprises at least one additional protein according to any of the proteins described herein.

### III. Methods of Predicting Tryptophan Oxidation

The invention herein also provides a method of predicting susceptibility to oxidation of a residue (such as tryptophan) of a protein in a liquid formulation. Molecule descriptors determined in silico by molecular dynamics (MD) simulation (such as all-atom MD simulation) using protein sequence information may be used to classify proteins in a liquid formulation as having residues (such as tryptophan residues) susceptible to oxidation. It is desirable to have a model, such as a computer learning algorithm, that is able to accurately predict or classify proteins in a liquid formulation as having residues susceptible to oxidation across a diverse array of molecule descriptors.

Methods of generating computer learning algorithms to predict susceptibility to oxidation of a residue (such as tryptophan) of a protein in a liquid formulation are provided. In some embodiments, the methods involve a) providing a training set comprising oxidation hotspot residues associated with i) values for a plurality of molecule descriptors (e.g. nearby aspartic acid sidechain oxygens, sidechain SASA, delta carbon SASA, nearby positive charge, backbone SASA, and the like) of the oxidation hotspot residues and ii) whether or not the oxidation hotspot residues are susceptible to oxidation; and b) applying the training set to a machine learning algorithm (e.g., a random decision forest), thereby training the machine learning algorithm to predict oxidation susceptibility. In some embodiments, the methods further comprise providing the machine learning algorithm (e.g., random decision forest) to predict the susceptibility to oxidation of one or more test residues having values for the plurality of molecule descriptors, comprising applying the plurality of molecule descriptors for each of the one or more test residues to the machine learning algorithm (e.g., random decision forest) and using the majority vote of the machine learning algorithm to classify the one or more test residues as being susceptible to oxidation or not. In some embodiments, the molecule descriptors are determined in silico by MD simulation. In some embodiments, oxidation of at least about 30% (such as at least about any of 35%, 40%, 45%, 50%, 55%, 60%, 65%, 70%, 75%, 80%, 85%, 90%, 95%, or more) of a residue in an oxidation assay indicates susceptibility to oxidation. In some embodiments, the protein is an antibody. In some embodiments, the antibody is a polyclonal antibody, a monoclonal antibody, a humanized antibody, a human antibody, a chimeric antibody, a multispecific antibody, or an antibody fragment. In some embodiments, the liquid formulation is an aqueous formulation.

In some embodiments, the machine learning algorithm is a random decision forest algorithm, in which bootstrap techniques are combined with random variable selection to grow multiple decision trees (Ho, T. K. Proceedings of the 3rd International Conference on Document Analysis and Recognition, Montreal, QC, 14-16 Aug. 1995. pp. 278-282; Ho, T. K. IEEE Transactions on Pattern Analysis and Machine Intelligence. 20(8) 832-844, 1998). These multiple decision trees are sometimes referred to herein as an ensemble of trees or the random decision forest. In some embodiments, the random decision forest comprises at least about 20 (such at least about any of 20, 30, 40, 50, 60, 70, 80, 90, 100, 125, 150, 175, 200, 250, 300, 400, 500, 600, 700, 800, 900, 1000, 2000, 3000, 4000, 5000, 10000 or more) decision trees (also referred to herein as “estimators”). In some embodiments, the number of variables randomly selected for consideration at each branch of each tree in the random decision forest (also referred to herein as “features”) is at least about 1 (such as at least about any of 1, 2, 3, 4, 5, 6, 7, 8, 9, 10, 11, 12, 13, 14, 15, or more). In some embodiments, the number of variables randomly selected for consideration at each branch of each tree in the random decision forest (also referred to herein as “tree depth”) is between about 1 to about 20 (such as between about any of 1 to 15, 1 to 10, 1 to 5, 5 to 20, 5, 15, or 5 to 10, including any ranges between these values). The variables include molecule descriptors of amino acid residues in a polypeptide chain. In some embodiments, the molecule descriptors are determined in silico by MD simulation. In some embodiments, the molecule descriptors include number of aspartic acid sidechain oxygens within 7 Å of test residue delta carbon, sidechain SASA (stdev), delta carbon SASA (stdev), total positive charge within 7 Å of test residue delta carbon (stdev), backbone SASA (stdev), test residue sidechain angles, packing density within 7 Å of test residue delta carbon, test residue backbone angles (stdev), SASA of pseudo-pi orbitals, backbone flexibility, and total negative charge within 7 Å of test residue delta carbon. In some embodiments, the maximum number of times the pool of observations is divided into sub-branches for each tree is at least about 2 (such as at least about any of 2, 3, 4, 5, 6, 7, 8, 9, 10, 15, 20, 25, 30, or more). In some embodiments, the maximum number of times the pool of observations is divided into sub-branches for each tree is at between about 2 to about 30 (such as at between about any of 2 to 20, 2 to 15, 2 to 10, 2 to 5, 5 to 30, 5 to 25, 5 to 20, 5 to 15, or 5 to 10, including any ranges between these values). Information about molecule descriptors of a test residue may be applied to the ensemble of trees to obtain a prediction about whether the test residue is susceptible to oxidation. The prediction is made by taking a majority vote of the predictions of all the trees in the ensemble.

In some embodiments, to determine the number of aspartic acid sidechain oxygens within 7 Å of test residue delta carbon using MD simulation, for each frame of each molecule simulation, all atoms within 7 Å of the delta carbon of the test residue are tracked, and of these atoms, those that are oxygen atoms on the sidechain of any aspartic acid residue are counted, and the final value is calculated as the time-average of this count over the duration of the simulation.

In some embodiments, to determine sidechain SASA using MD simulation, for each frame of each molecule simulation, points of a sphere centered on each atom in the simulation are generated by adding together each atomic radius with the radius of a water molecule, all points that are within the radii of neighboring spheres are eliminated, and the areas between all of the remaining points are summed to produce a value for SASA, and the final value of this descriptor is calculated as the average SASA of the test residue sidechain atoms over the duration of the simulation or the standard deviation of this calculation.

In some embodiments, to determine delta carbon SASA using MD simulation, for each frame of each simulation, the SASA of the test residue delta carbon is computed as described above, and the final value of this descriptor is calculated as the average SASA of the test residue delta carbon over the duration of the simulation or the standard deviation of this calculation.

In some embodiments, to determine total positive charge within 7 Å of test residue delta carbon using MD simulation, for each frame of each simulation, all atoms associated with a charged amino acid sidechain within 7 Å of the delta carbon of the test residue are tracked, the total positive charge of these atoms is added together, and the final value is calculated as the average of this quantity over the duration of the simulation or the standard deviation of this calculation.

In some embodiments, to determine backbone SASA using MD simulation, for each frame of each molecule simulation, the SASA of the backbone nitrogen atom of the test residue is computed as described above, and the final value of this descriptor is calculated as the average SASA of the backbone nitrogen atom over the duration of the simulation or the standard deviation of this calculation.

In some embodiments, to determine test residue sidechain angles using MD simulation, the chi1 and chi2 angle of the test residue sidechains are tracked through the simulation, and this descriptor is calculated as the percentage of the time that the test residue spends in an angle region most predictive of oxidation, wherein said angle region is determined by running many different simulations for many different residues of the same amino acid as the test residue, graphing all of the chi1 and chi2 angles over these simulations, and clustering commonly occurring angle combinations.

In some embodiments, packing density within 7 Å of test residue delta carbon is calculated using MD simulation as the time-averaged number of protein atoms within a sphere of radius 7 Å centered on the test residue delta carbon.

In some embodiments, test residue backbone angles is calculated using MD simulation as the average psi angle associated with the backbone of the test residue over the duration of the simulation or the standard deviation of this calculation.

In some embodiments, to determine the occupied volume of pseudo-pi orbitals using MD simulation, the sidechain of the test residue is treated as the base of a cylinder with a height appropriate to approximate the space occupied by test residue pi-orbitals, all atoms falling within the volume of the cylinder during the simulation are tracked, the total volume of all of the protein atoms falling within the volume of the cylinder is calculated for each frame of the simulations, and the final value is calculated as the time-averaged volume of the test residue pi-orbitals that were occupied by other protein atoms.

In some embodiments, to determine backbone flexibility using MD simulation, the root mean squared fluctuation of the backbone nitrogen of the test residue is calculated over each simulation. Each frame in the simulation is aligned, the distance traveled by each nitrogen atom is calculated for each frame, this distance for each frame is squared, the average of this squared distance across all frames is determined, and the final value of this descriptor is calculated as the square root of this average of the squared distance.

In some embodiments, to determine total negative charge within 7 Å of test residue delta carbon using MD simulation, for each frame of each simulation, all atoms associated with a charged amino acid sidechain within 7 Å of the delta carbon of the test residue are tracked, the total negative charge of these atoms is added together, and the final value is calculated as the time-average of this quantity over the duration of the simulation.

For example, in some embodiments, there is provided a method of generating a random decision forest for predicting whether a test residue of a protein in a liquid formulation is susceptible to oxidation comprising a) providing a training set comprising oxidation hotspot residues, wherein each residue is associated with i) values for a plurality of molecule descriptors of the residue and ii) whether the residue is susceptible to oxidation; and b) applying the training set to a random decision forest, thereby training the random decision forest to predict oxidation susceptibility, wherein the number of individual decision trees in the random decision forest is at least about 20 (such at least about any of 20, 30, 40, 50, 60, 70, 80, 90, 100, 125, 150, 175, 200, 250, 300, 400, 500, 600, 700, 800, 900, 1000, 2000, 3000, 4000, 5000, 10000 or more), the maximum number of variables randomly selected for consideration at each branch of each decision tree in the random decision forest is at least about 1 (such as at least about any of 1, 2, 3, 4, 5, 6, 7, 8, 9, 10, 11, 12, 13, 14, 15, or more), and the maximum number of times the pool of observations is divided into sub-branches for each tree is at least about 2 (such as at least about any of 2, 3, 4, 5, 6, 7, 8, 9, 10, 15, 20, 25, 30, or more). In some embodiments, the plurality of molecule descriptors includes number of aspartic acid sidechain oxygens within 7 Å of test residue delta carbon, sidechain SASA (stdev), delta carbon SASA (stdev), total positive charge within 7 Å of test residue delta carbon (stdev), backbone SASA (stdev), test residue sidechain angles, packing density within 7 Å of test residue delta carbon, test residue backbone angles (stdev), SASA of pseudo-pi orbitals, backbone flexibility, and total negative charge within 7 Å of test residue delta carbon. In some embodiments, the plurality of molecule descriptors includes number of aspartic acid sidechain oxygens within 7 Å of test residue delta carbon, sidechain SASA (stdev), delta carbon SASA (stdev), total positive charge within 7 Å of test residue delta carbon (stdev), backbone SASA (stdev), and test residue sidechain angles. In some embodiments, the plurality of molecule descriptors comprises between 2 and 11 (such as any of 2, 3, 4, 5, 6, 7, 8, 9, 10, or 11) molecule descriptors. In some embodiments, the molecule descriptors are determined based on an amino acid sequence of the protein comprising the test residue, such as an Fv region when the protein is an antibody. In some embodiments, the molecule descriptors are determined in silico by MD simulation using parameters for a protein in a liquid formulation. In some embodiments, the test residue and the oxidation hotspot residues are residues of the same amino acid (e.g., they are all tryptophan residues). In some embodiments, the test residue and the oxidation hotspot residues are tryptophan residues. In some embodiments, oxidation of at least about 30% (such as at least about any of 35%, 40%, 45%, 50%, 55%, 60%, 65%, 70%, 75%, 80%, 85%, 90%, 95%, or more) of a residue in an oxidation assay indicates susceptibility to oxidation. In some embodiments, the protein is an antibody. In some embodiments, the antibody is a polyclonal antibody, a monoclonal antibody, a humanized antibody, a human antibody, a chimeric antibody, a multispecific antibody, or an antibody fragment. In some embodiments, the liquid formulation is an aqueous formulation.

In some embodiments, there is provided a method of predicting whether a test residue of a protein in a liquid formulation is susceptible to oxidation comprising a) determining values for a plurality of molecule descriptors of the test residue; and b) applying the plurality of molecule descriptors of the test residue to a random decision forest trained on the plurality of molecule descriptors to predict oxidation susceptibility, wherein the majority vote of the random decision forest classifies the test residue as being susceptible to oxidation or not. In some embodiments, the random decision forest was trained by providing a training set comprising oxidation hotspot residues, wherein each residue is associated with i) values for the plurality of molecule descriptors for the residue; and ii) whether the residue is susceptible to oxidation; and applying the training set to a random decision forest, thereby training the random decision forest to predict oxidation susceptibility, wherein the number of individual decision trees in the random decision forest is at least about 20 (such at least about any of 20, 30, 40, 50, 60, 70, 80, 90, 100, 125, 150, 175, 200, 250, 300, 400, 500, 600, 700, 800, 900, 1000, 2000, 3000, 4000, 5000, 10000 or more), the maximum number of variables randomly selected for consideration at each branch of each decision tree in the random decision forest is at least about 1 (such as at least about any of 1, 2, 3, 4, 5, 6, 7, 8, 9, 10, 11, 12, 13, 14, 15, or more), and the maximum number of times the pool of observations is divided into sub-branches for each tree is at least about 2 (such as at least about any of 2, 3, 4, 5, 6, 7, 8, 9, 10, 15, 20, 25, 30, or more). In some embodiments, the plurality of molecule descriptors includes number of aspartic acid sidechain oxygens within 7 Å of test residue delta carbon, sidechain SASA (stdev), delta carbon SASA (stdev), total positive charge within 7 Å of test residue delta carbon (stdev), backbone SASA (stdev), test residue sidechain angles, packing density within 7 Å of test residue delta carbon, test residue backbone angles (stdev), SASA of pseudo-pi orbitals, backbone flexibility, and total negative charge within 7 Å of test residue delta carbon. In some embodiments, the plurality of molecule descriptors includes number of aspartic acid sidechain oxygens within 7 Å of test residue delta carbon, sidechain SASA (stdev), delta carbon SASA (stdev), total positive charge within 7 Å of test residue delta carbon (stdev), backbone SASA (stdev), and test residue sidechain angles. In some embodiments, the plurality of molecule descriptors comprises between 2 and 11 (such as any of 2, 3, 4, 5, 6, 7, 8, 9, 10, or 11) molecule descriptors. In some embodiments, the molecule descriptors are determined based on an amino acid sequence of the protein comprising the test residue, such as an Fv region when the protein is an antibody. In some embodiments, values for the molecule descriptors are determined in silico by MD simulation using parameters for a protein in a liquid formulation. In some embodiments, the test residue and the oxidation hotspot residues are residues of the same amino acid (e.g., they are all tryptophan residues). In some embodiments, the test residue and the oxidation hotspot residues are tryptophan residues. In some embodiments, oxidation of at least about 30% (such as at least about any of 35%, 40%, 45%, 50%, 55%, 60%, 65%, 70%, 75%, 80%, 85%, 90%, 95%, or more) of a residue in an oxidation assay indicates susceptibility to oxidation. In some embodiments, the protein is an antibody. In some embodiments, the antibody is a polyclonal antibody, a monoclonal antibody, a humanized antibody, a human antibody, a chimeric antibody, a multispecific antibody, or an antibody fragment. In some embodiments, the liquid formulation is an aqueous formulation.

In some embodiments, there is provided a method of predicting whether a test tryptophan residue of a protein in a liquid formulation is susceptible to oxidation comprising a) determining values for a plurality of molecule descriptors of the test tryptophan residue; and b) applying the plurality of molecule descriptors of the test tryptophan residue to a random decision forest trained on the plurality of molecule descriptors to predict oxidation susceptibility, wherein the majority vote of the random decision forest classifies the test tryptophan residue as being susceptible to oxidation or not. In some embodiments, the random decision forest was trained by providing a training set comprising tryptophan oxidation hotspot residues, wherein each residue is associated with i) values for the plurality of molecule descriptors for the residue; and ii) whether the residue is susceptible to oxidation; and applying the training set to a random decision forest, thereby training the random decision forest to predict tryptophan oxidation susceptibility, wherein the number of individual decision trees in the random decision forest is at least about 20 (such at least about any of 20, 30, 40, 50, 60, 70, 80, 90, 100, 125, 150, 175, 200, 250, 300, 400, 500, 600, 700, 800, 900, 1000, 2000, 3000, 4000, 5000, 10000 or more), the maximum number of variables randomly selected for consideration at each branch of each decision tree in the random decision forest is at least about 1 (such as at least about any of 1, 2, 3, 4, 5, 6, 7, 8, 9, 10, 11, 12, 13, 14, 15, or more), and the maximum number of times the pool of observations is divided into sub-branches for each tree is at least about 2 (such as at least about any of 2, 3, 4, 5, 6, 7, 8, 9, 10, 15, 20, 25, 30, or more). In some embodiments, the plurality of molecule descriptors includes number of aspartic acid sidechain oxygens within 7 Å of tryptophan delta carbon, sidechain SASA (stdev), delta carbon SASA (stdev), total positive charge within 7 Å of tryptophan delta carbon (stdev), backbone SASA (stdev), tryptophan sidechain angles, packing density within 7 Å of tryptophan delta carbon, tryptophan backbone angles (stdev), SASA of pseudo-pi orbitals, backbone flexibility, and total negative charge within 7 Å of tryptophan delta carbon. In some embodiments, the plurality of molecule descriptors includes number of aspartic acid sidechain oxygens within 7 Å of tryptophan delta carbon, sidechain SASA (stdev), delta carbon SASA (stdev), total positive charge within 7 Å of tryptophan delta carbon (stdev), backbone SASA (stdev), and tryptophan sidechain angles. In some embodiments, the plurality of molecule descriptors comprises between 2 and 11 (such as any of 2, 3, 4, 5, 6, 7, 8, 9, 10, or 11) molecule descriptors. In some embodiments, the molecule descriptors are determined based on an amino acid sequence of the protein comprising the test tryptophan, such as an Fv region when the protein is an antibody. In some embodiments, values for the molecule descriptors are determined in silico by MD simulation using parameters for a protein in a liquid formulation. In some embodiments, oxidation of at least about 30% (such as at least about any of 35%, 40%, 45%, 50%, 55%, 60%, 65%, 70%, 75%, 80%, 85%, 90%, 95%, or more) of a residue in an oxidation assay indicates susceptibility to oxidation. In some embodiments, the protein is an antibody. In some embodiments, the antibody is a polyclonal antibody, a monoclonal antibody, a humanized antibody, a human antibody, a chimeric antibody, a multispecific antibody, or an antibody fragment. In some embodiments, the liquid formulation is an aqueous formulation.

In some embodiments, there is provided a method of determining if a protein in a liquid formulation comprises a tryptophan residue susceptible to oxidation comprising a) determining values for a plurality of molecule descriptors for each tryptophan residue in the protein; and b) applying the plurality of molecule descriptors to a random decision forest trained on the plurality of molecule descriptors to predict oxidation susceptibility, wherein a majority vote of the random decision forest for each tryptophan residue classifies the residue as being susceptible to oxidation or not, and wherein the protein is determined to comprise a tryptophan residue susceptible to oxidation if the random decision forest classifies at least one tryptophan residue as being susceptible to oxidation. In some embodiments, the random decision forest was trained by providing a training set comprising tryptophan oxidation hotspot residues, wherein each residue is associated with i) values for the plurality of molecule descriptors for the residue; and ii) whether the residue is susceptible to oxidation; and applying the training set to a random decision forest, thereby training the random decision forest to predict tryptophan oxidation susceptibility, wherein the number of individual decision trees in the random decision forest is at least about 20 (such at least about any of 20, 30, 40, 50, 60, 70, 80, 90, 100, 125, 150, 175, 200, 250, 300, 400, 500, 600, 700, 800, 900, 1000, 2000, 3000, 4000, 5000, 10000 or more), the maximum number of variables randomly selected for consideration at each branch of each decision tree in the random decision forest is at least about 1 (such as at least about any of 1, 2, 3, 4, 5, 6, 7, 8, 9, 10, 11, 12, 13, 14, 15, or more), and the maximum number of times the pool of observations is divided into sub-branches for each tree is at least about 2 (such as at least about any of 2, 3, 4, 5, 6, 7, 8, 9, 10, 15, 20, 25, 30, or more). In some embodiments, the plurality of molecule descriptors includes number of aspartic acid sidechain oxygens within 7 Å of tryptophan delta carbon, sidechain SASA (stdev), delta carbon SASA (stdev), total positive charge within 7 Å of tryptophan delta carbon (stdev), backbone SASA (stdev), tryptophan sidechain angles, packing density within 7 Å of tryptophan delta carbon, tryptophan backbone angles (stdev), SASA of pseudo-pi orbitals, backbone flexibility, and total negative charge within 7 Å of tryptophan delta carbon. In some embodiments, the plurality of molecule descriptors includes number of aspartic acid sidechain oxygens within 7 Å of tryptophan delta carbon, sidechain SASA (stdev), delta carbon SASA (stdev), total positive charge within 7 Å of tryptophan delta carbon (stdev), backbone SASA (stdev), and tryptophan sidechain angles. In some embodiments, the plurality of molecule descriptors comprises between 2 and 11 (such as any of 2, 3, 4, 5, 6, 7, 8, 9, 10, or 11) molecule descriptors. In some embodiments, the molecule descriptors of each tryptophan residue are determined based on an amino acid sequence of the protein comprising the tryptophan residue, such as an Fv region when the protein is an antibody. In some embodiments, values for the molecule descriptors are determined in silico by MD simulation using parameters for a protein in a liquid formulation. In some embodiments, oxidation of at least about 30% (such as at least about any of 35%, 40%, 45%, 50%, 55%, 60%, 65%, 70%, 75%, 80%, 85%, 90%, 95%, or more) of a residue in an oxidation assay indicates susceptibility to oxidation. In some embodiments, the protein is an antibody. In some embodiments, the antibody is a polyclonal antibody, a monoclonal antibody, a humanized antibody, a human antibody, a chimeric antibody, a multispecific antibody, or an antibody fragment. In some embodiments, the liquid formulation is an aqueous formulation.

### IV. Methods of Reducing Oxidation

The invention herein also provides a method of reducing oxidation of a protein in a liquid formulation comprising adding an amount of NAT that prevents oxidation of the protein in the liquid formulation. In some embodiments, the protein is susceptible to oxidation. In some embodiments, methionine, cysteine, histidine, tryptophan, and/or tyrosine in the protein is susceptible to oxidation. In some embodiments, tryptophan in the protein is susceptible to oxidation. In some embodiments, the protein comprises at least one tryptophan residue with a solvent-accessible surface area (SASA) greater than about 50 Å² to about 250 Å² (such as greater than about any of 50, 60, 70, 80, 90, 100, 120, 140, 160, 180, 200, 225, or 250 Å², including any ranges between these values). In some embodiments, the SASA is greater than about 80 Å². In some embodiments, the protein comprises at least one tryptophan residue with a SASA greater than about 15% to about 45% (such as greater than about any of 15, 20, 25, 30, 35, 40, or 45%). In some embodiments, the SASA is greater than about 30%. In some embodiments, SASA of a tryptophan residue is measured at a pH range from about 4.0 to about 8.5. In some embodiments, SASA of a tryptophan residue is measured at a temperature ranging from about 5° C. to about 40° C. In some embodiments, SASA of a tryptophan residue is measured at a salt concentration ranging from about 0 mM to about 500 mM. In some embodiments, SASA of a tryptophan residue is measured at a pH of about 5.0 to about 7.5, a temperature of about 5° C. to about 25° C. and a salt concentration of about 0 mM to about 200 mM. In some embodiments, the SASA is determined in silico by all-atom molecular dynamics simulation. In some embodiments, the protein comprises at least one tryptophan residue predicted to be susceptible to oxidation by a machine learning algorithm trained on associations of tryptophan residue oxidation susceptibility with a plurality of molecule descriptors of the tryptophan residue based on MD simulations. In some embodiments, the amount of NAT added to the formulation is from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values), or up to the highest concentration that the NAT is soluble in the formulation. In some embodiments, the amount of NAT added to the formulation is about 1 mM. In some embodiments, the NAT prevents oxidation of one or more tryptophan amino acids in the protein. In some embodiments, the oxidation of the protein is reduced by about 40% to about 100% (such as by about any of 40%, 45%, 50%, 55%, 60%, 65%, 70%, 75%, 80%, 85%, 90%, 95%, 96%, 97%, 98%, 99%, or 100%, including any ranges between these values). In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of the protein is oxidized. In some embodiments, the NAT prevents oxidation of the protein by a reactive oxygen species (ROS). In a further embodiment, the reactive oxygen species is selected from the group consisting of a singlet oxygen, a superoxide (O₂—), an alkoxyl radical, a peroxyl radical, a hydrogen peroxide (H₂O₂), a dihydrogen trioxide (H₂O₃), a hydrotrioxy radical (HO₃.), ozone (O₃), a hydroxyl radical, and an alkyl peroxide. In some embodiments, the protein (e.g., the antibody) concentration in the formulation is about 1 mg/mL to about 250 mg/mL. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the formulation further comprises one or more excipients selected from the group consisting of a stabilizer, a buffer, a surfactant, and a tonicity agent. For example, a formulation of the invention can comprise a monoclonal antibody, NAT as provided herein which prevents oxidation of the protein, and a buffer that maintains the pH of the formulation to a desirable level. In some embodiments, the formulation has a pH of about 4.5 to about 7.0. In some embodiments, the formulation is aqueous. In some embodiments, the formulation further comprises at least one additional protein according to any of the proteins described herein (e.g., the formulation is a co-formulation comprising two or more proteins).

In some embodiments, there is provided a method of reducing oxidation of a protein in a liquid formulation comprising adding an amount of NAT to the formulation that prevents oxidation of the protein, wherein the protein comprises at least one tryptophan residue with a SASA of greater than about 50 Å² to about 250 Å² (such as greater than about any of 50, 60, 70, 80, 90, 100, 120, 140, 160, 180, 200, 225, or 250 Å², including any ranges between these values). In some embodiments, the protein comprises at least one tryptophan residue with a SASA greater than about 80 Å². In some embodiments, the SASA is determined in silico by all-atom molecular dynamics simulation. In some embodiments, the amount of NAT added to the formulation is from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values), or up to the highest concentration that the NAT is soluble in the formulation. In some embodiments, the amount of NAT added to the formulation is about 1 mM. In some embodiments, the NAT prevents oxidation of one or more tryptophan amino acids in the protein. In some embodiments, the oxidation of the protein is reduced by about 40% to about 100% (such as by about any of 40%, 45%, 50%, 55%, 60%, 65%, 70%, 75%, 80%, 85%, 90%, 95%, 96%, 97%, 98%, 99%, or 100%, including any ranges between these values). In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of the protein is oxidized. In some embodiments, the NAT prevents oxidation of the protein by a reactive oxygen species (ROS). In some embodiments, the protein (e.g., the antibody) concentration in the formulation is about 1 mg/mL to about 250 mg/mL. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the antibody is derived from an IgG1 antibody sequence. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation. In some embodiments, the formulation further comprises at least one additional protein according to any of the proteins described herein.

In some embodiments, there is provided a method of reducing oxidation of a protein in a liquid formulation comprising determining the SASA values of tryptophan residues in the protein and adding an amount of NAT to the formulation that prevents oxidation of the protein if at least one tryptophan residue has a SASA of greater than about 50 Å² to about 250 Å² (such as greater than about any of 50, 60, 70, 80, 90, 100, 120, 140, 160, 180, 200, 225, or 250 Å², including any ranges between these values). In some embodiments, the protein comprises at least one tryptophan residue with a SASA greater than about 80 Å². In some embodiments, the SASA is determined in silico by all-atom molecular dynamics simulation. In some embodiments, the amount of NAT added to the formulation is from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values), or up to the highest concentration that the NAT is soluble in the formulation. In some embodiments, the amount of NAT added to the formulation is about 1 mM. In some embodiments, the NAT prevents oxidation of one or more tryptophan amino acids in the protein. In some embodiments, the oxidation of the protein is reduced by about 40% to about 100% (such as by about any of 40%, 45%, 50%, 55%, 60%, 65%, 70%, 75%, 80%, 85%, 90%, 95%, 96%, 97%, 98%, 99%, or 100%, including any ranges between these values). In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of the protein is oxidized. In some embodiments, the NAT prevents oxidation of the protein by a reactive oxygen species (ROS). In some embodiments, the protein (e.g., the antibody) concentration in the formulation is about 1 mg/mL to about 250 mg/mL. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the antibody is derived from an IgG1 antibody sequence. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation. In some embodiments, the formulation further comprises at least one additional protein according to any of the proteins described herein.

In some embodiments, there is provided a method of reducing oxidation of a protein in a liquid formulation comprising determining the SASA values of tryptophan residues in the protein and adding an amount of NAT to the formulation based on the number of tryptophan residues having a SASA of greater than about 50 Å² to about 250 Å² (such as greater than about any of 50, 60, 70, 80, 90, 100, 120, 140, 160, 180, 200, 225, or 250 Å², including any ranges between these values), wherein the amount of NAT added to the formulation prevents oxidation of the protein. In some embodiments, the SASA is determined in silico by all-atom molecular dynamics simulation. In some embodiments, the amount of NAT added to the formulation is from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values). In some embodiments, the amount of NAT added to the formulation is about 1 mM. In some embodiments, the NAT prevents oxidation of one or more tryptophan amino acids in the protein. In some embodiments, the protein has more than one tryptophan residues with a SASA greater than 85 Å² (or greater than 30%) and a sufficient amount of NAT is added to prevent oxidation of each tryptophan residue. In some embodiments, the oxidation of the protein is reduced by about 40% to about 100% (such as by about any of 40%, 45%, 50%, 55%, 60%, 65%, 70%, 75%, 80%, 85%, 90%, 95%, 96%, 97%, 98%, 99%, or 100%, including any ranges between these values). In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of the protein is oxidized. In some embodiments, the NAT prevents oxidation of the protein by a reactive oxygen species (ROS). In some embodiments, the protein (e.g., the antibody) concentration in the formulation is about 1 mg/mL to about 250 mg/mL. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the antibody is derived from an IgG1 antibody sequence. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation. In some embodiments, the formulation further comprises at least one additional protein according to any of the proteins described herein.

In some embodiments, there is provided a method of reducing oxidation of a protein in a liquid formulation comprising adding an amount of NAT to the formulation that prevents oxidation of the protein, wherein the protein comprises at least one tryptophan residue with a SASA of greater than about 15% to about 45% (such as greater than about any of 15, 20, 25, 30, 35, 40, or 45%). In some embodiments, the protein comprises at least one tryptophan residue with a SASA greater than about 30%. In some embodiments, the SASA is determined in silico by all-atom molecular dynamics simulation. In some embodiments, the amount of NAT added to the formulation is from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values), or up to the highest concentration that the NAT is soluble in the formulation. In some embodiments, the amount of NAT added to the formulation is about 1 mM. In some embodiments, the NAT prevents oxidation of one or more tryptophan amino acids in the protein. In some embodiments, the oxidation of the protein is reduced by about 40% to about 100% (such as by about any of 40%, 45%, 50%, 55%, 60%, 65%, 70%, 75%, 80%, 85%, 90%, 95%, 96%, 97%, 98%, 99%, or 100%, including any ranges between these values). In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of the protein is oxidized. In some embodiments, the NAT prevents oxidation of the protein by a reactive oxygen species (ROS). In some embodiments, the protein (e.g., the antibody) concentration in the formulation is about 1 mg/mL to about 250 mg/mL. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the antibody is derived from an IgG1 antibody sequence. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation. In some embodiments, the formulation further comprises at least one additional protein according to any of the proteins described herein.

In some embodiments, there is provided a method of reducing oxidation of a protein in a liquid formulation comprising determining the SASA values of tryptophan residues in the protein and adding an amount of NAT to the formulation that prevents oxidation of the protein if at least one tryptophan residue has a SASA of greater than about 15% to about 45% (such as greater than about any of 15, 20, 25, 30, 35, 40, or 45%). In some embodiments, the protein comprises at least one tryptophan residue with a SASA greater than about 30%. In some embodiments, the SASA is determined in silico by all-atom molecular dynamics simulation. In some embodiments, the amount of NAT added to the formulation is from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values), or up to the highest concentration that the NAT is soluble in the formulation. In some embodiments, the amount of NAT added to the formulation is about 1 mM. In some embodiments, the NAT prevents oxidation of one or more tryptophan amino acids in the protein. In some embodiments, the oxidation of the protein is reduced by about 40% to about 100% (such as by about any of 40%, 45%, 50%, 55%, 60%, 65%, 70%, 75%, 80%, 85%, 90%, 95%, 96%, 97%, 98%, 99%, or 100%, including any ranges between these values). In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of the protein is oxidized. In some embodiments, the NAT prevents oxidation of the protein by a reactive oxygen species (ROS). In some embodiments, the protein (e.g., the antibody) concentration in the formulation is about 1 mg/mL to about 250 mg/mL. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the antibody is derived from an IgG1 antibody sequence. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation. In some embodiments, the formulation further comprises at least one additional protein according to any of the proteins described herein.

In some embodiments, there is provided a method of reducing oxidation of a protein in a liquid formulation comprising determining the SASA values of tryptophan residues in the protein and adding an amount of NAT to the formulation based on the number of tryptophan residues having a SASA of greater than about 15% to about 45% (such as greater than about any of 15, 20, 25, 30, 35, 40, or 45%), wherein the amount of NAT added to the formulation prevents oxidation of the protein. In some embodiments, the SASA is determined in silico by all-atom molecular dynamics simulation. In some embodiments, the amount of NAT added to the formulation is from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values). In some embodiments, the amount of NAT added to the formulation is about 1 mM. In some embodiments, the NAT prevents oxidation of one or more tryptophan amino acids in the protein. In some embodiments, the oxidation of the protein is reduced by about 40% to about 100% (such as by about any of 40%, 45%, 50%, 55%, 60%, 65%, 70%, 75%, 80%, 85%, 90%, 95%, 96%, 97%, 98%, 99%, or 100%, including any ranges between these values). In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of the protein is oxidized. In some embodiments, the NAT prevents oxidation of the protein by a reactive oxygen species (ROS). In some embodiments, the protein (e.g., the antibody) concentration in the formulation is about 1 mg/mL to about 250 mg/mL. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the antibody is derived from an IgG1 antibody sequence. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation. In some embodiments, the formulation further comprises at least one additional protein according to any of the proteins described herein.

SASA can be calculated using the in silico all-atom molecular dynamics simulation method described in Sharma, V. et al. (supra), described in more detail below in the Examples.

In some embodiments, there is provided a method of reducing oxidation of a protein in a liquid formulation comprising adding an amount of an anti-oxidation agent to the formulation that prevents oxidation of the protein, wherein the protein comprises at least one tryptophan residue predicted to be susceptible to oxidation by a machine learning algorithm trained on associations of tryptophan residue oxidation susceptibility with a plurality of molecule descriptors of the tryptophan residue based on MD simulations. In some embodiments, the anti-oxidation agent is N-acetyltryptophan (NAT). In some embodiments, the amount of NAT added to the formulation is from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values), or up to the highest concentration that the NAT is soluble in the formulation. In some embodiments, the amount of NAT added to the formulation is about 1 mM. In some embodiments, the NAT prevents oxidation of one or more tryptophan amino acids in the protein. In some embodiments, the oxidation of the protein is reduced by about 40% to about 100% (such as by about any of 40%, 45%, 50%, 55%, 60%, 65%, 70%, 75%, 80%, 85%, 90%, 95%, 96%, 97%, 98%, 99%, or 100%, including any ranges between these values). In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of the protein is oxidized. In some embodiments, the NAT prevents oxidation of the protein by a reactive oxygen species (ROS). In some embodiments, the protein (e.g., the antibody) concentration in the formulation is about 1 mg/mL to about 250 mg/mL. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation. In some embodiments, the formulation further comprises at least one additional protein according to any of the proteins described herein. In some embodiments, the machine learning algorithm is a random decision forest according to any of the random decision forests described above. In some embodiments, the random decision forest was trained with at least about 20 (such at least about any of 20, 30, 40, 50, 60, 70, 80, 90, 100, 125, 150, 175, 200, 250, 300, 400, 500, 600, 700, 800, 900, 1000, 2000, 3000, 4000, 5000, 10000 or more) estimators, at least about 1 (such as at least about any of 1, 2, 3, 4, 5, 6, 7, 8, 9, 10, 11, 12, 13, 14, 15, or more) features, and a tree depth of at least about 2 (such as at least about any of 2, 3, 4, 5, 6, 7, 8, 9, 10, 15, 20, 25, 30, or more). In some embodiments, the plurality of molecule descriptors includes number of aspartic acid sidechain oxygens within 7 Å of tryptophan delta carbon, sidechain SASA (stdev), delta carbon SASA (stdev), total positive charge within 7 Å of tryptophan delta carbon (stdev), backbone SASA (stdev), tryptophan sidechain angles, packing density within 7 Å of tryptophan delta carbon, tryptophan backbone angles (stdev), SASA of pseudo-pi orbitals, backbone flexibility, and total negative charge within 7 Å of tryptophan delta carbon. In some embodiments, the plurality of molecule descriptors comprises between 2 and 11 (such as any of 2, 3, 4, 5, 6, 7, 8, 9, 10, or 11) molecule descriptors. In some embodiments, values for the tryptophan molecule descriptors are determined in silico by MD simulation using parameters for a protein in a liquid formulation. In some embodiments, oxidation of at least about 30% (such as at least about any of 35%, 40%, 45%, 50%, 55%, 60%, 65%, 70%, 75%, 80%, 85%, 90%, 95%, or more) of a residue in an oxidation assay indicates susceptibility to oxidation.

In some embodiments, there is provided a method of reducing oxidation of a protein in a liquid formulation comprising adding an amount of NAT to the formulation that prevents oxidation of the protein, wherein the protein comprises at least one tryptophan residue predicted to be susceptible to oxidation by a machine learning algorithm trained on associations of tryptophan residue oxidation susceptibility with a plurality of molecule descriptors of the tryptophan residue based on MD simulations. In some embodiments, the amount of NAT added to the formulation is from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values), or up to the highest concentration that the NAT is soluble in the formulation. In some embodiments, the amount of NAT added to the formulation is about 1 mM. In some embodiments, the NAT prevents oxidation of one or more tryptophan amino acids in the protein. In some embodiments, the oxidation of the protein is reduced by about 40% to about 100% (such as by about any of 40%, 45%, 50%, 55%, 60%, 65%, 70%, 75%, 80%, 85%, 90%, 95%, 96%, 97%, 98%, 99%, or 100%, including any ranges between these values). In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of the protein is oxidized. In some embodiments, the NAT prevents oxidation of the protein by a reactive oxygen species (ROS). In some embodiments, the protein (e.g., the antibody) concentration in the formulation is about 1 mg/mL to about 250 mg/mL. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation. In some embodiments, the formulation further comprises at least one additional protein according to any of the proteins described herein. In some embodiments, the machine learning algorithm is a random decision forest according to any of the random decision forests described above. In some embodiments, the random decision forest was trained with at least about 20 (such at least about any of 20, 30, 40, 50, 60, 70, 80, 90, 100, 125, 150, 175, 200, 250, 300, 400, 500, 600, 700, 800, 900, 1000, 2000, 3000, 4000, 5000, 10000 or more) estimators, at least about 1 (such as at least about any of 1, 2, 3, 4, 5, 6, 7, 8, 9, 10, 11, 12, 13, 14, 15, or more) features, and a tree depth of at least about 2 (such as at least about any of 2, 3, 4, 5, 6, 7, 8, 9, 10, 15, 20, 25, 30, or more). In some embodiments, the plurality of molecule descriptors includes number of aspartic acid sidechain oxygens within 7 Å of tryptophan delta carbon, sidechain SASA (stdev), delta carbon SASA (stdev), total positive charge within 7 Å of tryptophan delta carbon (stdev), backbone SASA (stdev), tryptophan sidechain angles, packing density within 7 Å of tryptophan delta carbon, tryptophan backbone angles (stdev), SASA of pseudo-pi orbitals, backbone flexibility, and total negative charge within 7 Å of tryptophan delta carbon. In some embodiments, the plurality of molecule descriptors comprises between 2 and 11 (such as any of 2, 3, 4, 5, 6, 7, 8, 9, 10, or 11) molecule descriptors. In some embodiments, values for the tryptophan molecule descriptors are determined in silico by MD simulation using parameters for a protein in a liquid formulation. In some embodiments, oxidation of at least about 30% (such as at least about any of 35%, 40%, 45%, 50%, 55%, 60%, 65%, 70%, 75%, 80%, 85%, 90%, 95%, or more) of a residue in an oxidation assay indicates susceptibility to oxidation.

In some embodiments, there is provided a method of reducing oxidation of a protein in a liquid formulation comprising adding an amount of NAT to the formulation based on the number of tryptophan residues predicted to be susceptible to oxidation by a machine learning algorithm trained on associations of tryptophan residue oxidation susceptibility with a plurality of molecule descriptors of the tryptophan residue based on MD simulations. In some embodiments, the amount of NAT added to the formulation is from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values), or up to the highest concentration that the NAT is soluble in the formulation. In some embodiments, the amount of NAT added to the formulation is about 1 mM. In some embodiments, the NAT prevents oxidation of one or more tryptophan amino acids in the protein. In some embodiments, the oxidation of the protein is reduced by about 40% to about 100% (such as by about any of 40%, 45%, 50%, 55%, 60%, 65%, 70%, 75%, 80%, 85%, 90%, 95%, 96%, 97%, 98%, 99%, or 100%, including any ranges between these values). In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of the protein is oxidized. In some embodiments, the NAT prevents oxidation of the protein by a reactive oxygen species (ROS). In some embodiments, the protein (e.g., the antibody) concentration in the formulation is about 1 mg/mL to about 250 mg/mL. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation. In some embodiments, the formulation further comprises at least one additional protein according to any of the proteins described herein. In some embodiments, the machine learning algorithm is a random decision forest according to any of the random decision forests described above. In some embodiments, the random decision forest was trained with at least about 20 (such at least about any of 20, 30, 40, 50, 60, 70, 80, 90, 100, 125, 150, 175, 200, 250, 300, 400, 500, 600, 700, 800, 900, 1000, 2000, 3000, 4000, 5000, 10000 or more) estimators, at least about 1 (such as at least about any of 1, 2, 3, 4, 5, 6, 7, 8, 9, 10, 11, 12, 13, 14, 15, or more) features, and a tree depth of at least about 2 (such as at least about any of 2, 3, 4, 5, 6, 7, 8, 9, 10, 15, 20, 25, 30, or more). In some embodiments, the plurality of molecule descriptors includes number of aspartic acid sidechain oxygens within 7 Å of tryptophan delta carbon, sidechain SASA (stdev), delta carbon SASA (stdev), total positive charge within 7 Å of tryptophan delta carbon (stdev), backbone SASA (stdev), tryptophan sidechain angles, packing density within 7 Å of tryptophan delta carbon, tryptophan backbone angles (stdev), SASA of pseudo-pi orbitals, backbone flexibility, and total negative charge within 7 Å of tryptophan delta carbon. In some embodiments, the plurality of molecule descriptors comprises between 2 and 11 (such as any of 2, 3, 4, 5, 6, 7, 8, 9, 10, or 11) molecule descriptors. In some embodiments, values for the tryptophan molecule descriptors are determined in silico by MD simulation using parameters for a protein in a liquid formulation. In some embodiments, oxidation of at least about 30% (such as at least about any of 35%, 40%, 45%, 50%, 55%, 60%, 65%, 70%, 75%, 80%, 85%, 90%, 95%, or more) of a residue in an oxidation assay indicates susceptibility to oxidation.

In some embodiments, there is provided a method of reducing oxidation of a protein in a liquid formulation comprising introducing an amino acid substitution in the protein to replace one or more tryptophan residues predicted to be susceptible to oxidation with amino acid residues that are not subject to oxidation, wherein the prediction is by a machine learning algorithm trained on associations of tryptophan residue oxidation susceptibility with a plurality of molecule descriptors of the tryptophan residue based on MD simulations. In some embodiments, the one or more tryptophan residues are each replaced by a residue independently selected from the group consisting of tyrosine, phenylalanine, leucine, isoleucine, alanine, and valine. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation. In some embodiments, the formulation further comprises at least one additional protein according to any of the proteins described herein. In some embodiments, the machine learning algorithm is a random decision forest according to any of the random decision forests described above. In some embodiments, the random decision forest was trained with at least about 20 (such at least about any of 20, 30, 40, 50, 60, 70, 80, 90, 100, 125, 150, 175, 200, 250, 300, 400, 500, 600, 700, 800, 900, 1000, 2000, 3000, 4000, 5000, 10000 or more) estimators, at least about 1 (such as at least about any of 1, 2, 3, 4, 5, 6, 7, 8, 9, 10, 11, 12, 13, 14, 15, or more) features, and a tree depth of at least about 2 (such as at least about any of 2, 3, 4, 5, 6, 7, 8, 9, 10, 15, 20, 25, 30, or more). In some embodiments, the plurality of molecule descriptors includes number of aspartic acid sidechain oxygens within 7 Å of tryptophan delta carbon, sidechain SASA (stdev), delta carbon SASA (stdev), total positive charge within 7 Å of tryptophan delta carbon (stdev), backbone SASA (stdev), tryptophan sidechain angles, packing density within 7 Å of tryptophan delta carbon, tryptophan backbone angles (stdev), SASA of pseudo-pi orbitals, backbone flexibility, and total negative charge within 7 Å of tryptophan delta carbon. In some embodiments, the plurality of molecule descriptors comprises between 2 and 11 (such as any of 2, 3, 4, 5, 6, 7, 8, 9, 10, or 11) molecule descriptors. In some embodiments, values for the tryptophan molecule descriptors are determined in silico by MD simulation using parameters for a protein in a liquid formulation. In some embodiments, oxidation of at least about 30% (such as at least about any of 35%, 40%, 45%, 50%, 55%, 60%, 65%, 70%, 75%, 80%, 85%, 90%, 95%, or more) of a residue in an oxidation assay indicates susceptibility to oxidation.

### V. Methods of Screening

The invention herein also provides a method of screening a liquid formulation for reduced oxidation of a protein. In some embodiments, the protein is susceptible to oxidation. In some embodiments, methionine, cysteine, histidine, tryptophan, and/or tyrosine in the protein is susceptible to oxidation. In some embodiments, tryptophan in the protein is susceptible to oxidation. In some embodiments, the protein comprises at least one tryptophan residue with a solvent-accessible surface area (SASA) greater than about 50 Å² to about 250 Å² (such as greater than about any of 50, 60, 70, 80, 90, 100, 120, 140, 160, 180, 200, 225, or 250 Å², including any ranges between these values). In some embodiments, the SASA is greater than about 80 Å². In some embodiments, the protein comprises at least one tryptophan residue with a SASA greater than about 15% to about 45% (such as greater than about any of 15, 20, 25, 30, 35, 40, or 45%). In some embodiments, the SASA is greater than about 30%. In some embodiments, tryptophan in the protein is predicted to be susceptible to oxidation by a machine learning algorithm trained on associations of tryptophan residue oxidation susceptibility with a plurality of molecule descriptors of the tryptophan residue based on MD simulations. In some embodiments, the oxidation of the protein is reduced by about 40% to about 100% (such as by about any of 40%, 45%, 50%, 55%, 60%, 65%, 70%, 75%, 80%, 85%, 90%, 95%, 96%, 97%, 98%, 99%, or 100%, including any ranges between these values). In some embodiments, no more than about 40% to about 0% (such as no more than about any of 40%, 35%, 30%, 25%, 20%, 15%, 10%, 5%, 4%, 3%, 2%, 1%, or 0%, including any ranges between these values) of the protein is oxidized. In some embodiments, the protein (e.g., the antibody) concentration in the formulation is about 1 mg/mL to about 250 mg/mL. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the antibody is derived from an IgG1 antibody sequence. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation.

In some embodiments, there is provided a method of screening a liquid formulation for reduced oxidation of a protein wherein the protein comprises at least one tryptophan residue with i) a SASA of greater than about 50 Å² to about 250 Å² (such as greater than about any of 50, 60, 70, 80, 90, 100, 120, 140, 160, 180, 200, 225, or 250 Å², including any ranges between these values); or ii) a SASA greater than about 15% to about 45% (such as greater than about any of 15, 20, 25, 30, 35, 40, or 45%), the method comprising a) adding from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values) of N-acetyl-tryptophan (NAT) to a liquid formulation comprising the protein, b) adding from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values) of 2,2′-azobis (2-aminopropane) dihydrochloride (AAPH) to the liquid formulation, c) incubating the liquid formulation comprising the protein, NAT and AAPH for about 10 hours to about 20 hours (such as about any of 10, 11, 12, 13, 14, 15, 16, 17, 18, 19, or 20 hours, including any ranges between these values) at about 35° C. to about 45° C. (such as about any of 35, 36, 37, 38, 39, 40, 41, 42, 43, 44, or 45° C., including any ranges between these values), and d) measuring the protein for oxidation of tryptophan residues in the protein, wherein a liquid formulation comprising an amount of NAT that results in no more than about 20% (such as no more than about any of 20, 15, 10, 5, 4, 3, 2, or 1%, including any ranges between these values) oxidation of tryptophan residues in the protein is a suitable formulation for reduced oxidation of the protein. In some embodiments, the SASA is determined in silico by all-atom molecular dynamics simulation. In some embodiments, the protein (e.g., the antibody) concentration in the liquid formulation is about 1 mg/mL to about 250 mg/mL. In some embodiments, the protein is a therapeutic protein. In some of the embodiments, the protein is an antibody. In some embodiments, the antibody is a polyclonal antibody, a monoclonal antibody, a humanized antibody, a human antibody, a chimeric antibody, a multispecific antibody, or an antibody fragment. In some embodiments, the antibody is derived from an IgG1 antibody sequence. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation.

In some embodiments, there is provided a method of screening a liquid formulation for reduced oxidation of a protein comprising a) determining the SASA values of tryptophan residues in the protein, b) adding an amount of NAT to the liquid formulation based on the number of tryptophan residues having i) a SASA of greater than about 50 Å² to about 250 Å² (such as greater than about any of 50, 60, 70, 80, 90, 100, 120, 140, 160, 180, 200, 225, or 250 Å², including any ranges between these values); or ii) a SASA greater than about 15% to about 45% (such as greater than about any of 15, 20, 25, 30, 35, 40, or 45%), c) adding from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values) of 2,2′-azobis (2-aminopropane) dihydrochloride (AAPH) to the liquid formulation, d) incubating the liquid formulation comprising the protein, N-acetyl-tryptophan and AAPH for about 10 hours to about 20 hours (such as about any of 10, 11, 12, 13, 14, 15, 16, 17, 18, 19, or 20 hours, including any ranges between these values) at about 35° C. to about 45° C. (such as about any of 35, 36, 37, 38, 39, 40, 41, 42, 43, 44, or 45° C., including any ranges between these values), and e) measuring the protein for oxidation of tryptophan residues in the protein, wherein a liquid formulation comprising an amount of NAT that results in no more than about 20% (such as no more than about any of 20, 15, 10, 5, 4, 3, 2, or 1%, including any ranges between these values) oxidation of tryptophan residues in the protein is a suitable formulation for reduced oxidation of the protein. In some embodiments, the SASA is determined in silico by all-atom molecular dynamics simulation. In some embodiments, the protein (e.g., the antibody) concentration in the liquid formulation is about 1 mg/mL to about 250 mg/mL. In some embodiments, the protein is a therapeutic protein. In some of the embodiments, the protein is an antibody. In some embodiments, the antibody is a polyclonal antibody, a monoclonal antibody, a humanized antibody, a human antibody, a chimeric antibody, a multispecific antibody, or an antibody fragment. In some embodiments, the antibody is derived from an IgG1 antibody sequence. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation.

In some embodiments, there is provided a method of screening a liquid formulation for reduced oxidation of a protein wherein the protein comprises at least one tryptophan residue predicted to be susceptible to oxidation by a machine learning algorithm trained on associations of tryptophan residue oxidation susceptibility with a plurality of molecule descriptors of the tryptophan residue based on MD simulations, the method comprising a) adding from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values) of N-acetyl-tryptophan (NAT) to a liquid formulation comprising the protein, b) adding from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values) of 2,2′-azobis (2-aminopropane) dihydrochloride (AAPH) to the liquid formulation, c) incubating the liquid formulation comprising the protein, NAT and AAPH for about 10 hours to about 20 hours (such as about any of 10, 11, 12, 13, 14, 15, 16, 17, 18, 19, or 20 hours, including any ranges between these values) at about 35° C. to about 45° C. (such as about any of 35, 36, 37, 38, 39, 40, 41, 42, 43, 44, or 45° C., including any ranges between these values), and d) measuring the protein for oxidation of tryptophan residues in the protein, wherein a liquid formulation comprising an amount of NAT that results in no more than about 20% (such as no more than about any of 20, 15, 10, 5, 4, 3, 2, or 1%, including any ranges between these values) oxidation of tryptophan residues in the protein is a suitable formulation for reduced oxidation of the protein. In some embodiments, the protein (e.g., the antibody) concentration in the liquid formulation is about 1 mg/mL to about 250 mg/mL. In some embodiments, the protein is a therapeutic protein. In some of the embodiments, the protein is an antibody. In some embodiments, the antibody is a polyclonal antibody, a monoclonal antibody, a humanized antibody, a human antibody, a chimeric antibody, a multispecific antibody, or an antibody fragment. In some embodiments, the formulation further comprises one or more excipients selected from the group consisting of a stabilizer, a buffer, a surfactant, and a tonicity agent. In some embodiments, the formulation has a pH of about 4.5 to about 7.0. In some embodiments, the liquid formulation is an aqueous formulation. In some embodiments, the machine learning algorithm is a random decision forest according to any of the random decision forests described above. In some embodiments, the random decision forest was trained with at least about 20 (such at least about any of 20, 30, 40, 50, 60, 70, 80, 90, 100, 125, 150, 175, 200, 250, 300, 400, 500, 600, 700, 800, 900, 1000, 2000, 3000, 4000, 5000, 10000 or more) estimators, at least about 1 (such as at least about any of 1, 2, 3, 4, 5, 6, 7, 8, 9, 10, 11, 12, 13, 14, 15, or more) features, and a tree depth of at least about 2 (such as at least about any of 2, 3, 4, 5, 6, 7, 8, 9, 10, 15, 20, 25, 30, or more). In some embodiments, the plurality of molecule descriptors includes number of aspartic acid sidechain oxygens within 7 Å of tryptophan delta carbon, sidechain SASA (stdev), delta carbon SASA (stdev), total positive charge within 7 Å of tryptophan delta carbon (stdev), backbone SASA (stdev), tryptophan sidechain angles, packing density within 7 Å of tryptophan delta carbon, tryptophan backbone angles (stdev), SASA of pseudo-pi orbitals, backbone flexibility, and total negative charge within 7 Å of tryptophan delta carbon. In some embodiments, the plurality of molecule descriptors comprises between 2 and 11 (such as any of 2, 3, 4, 5, 6, 7, 8, 9, 10, or 11) molecule descriptors. In some embodiments, values for the tryptophan molecule descriptors are determined in silico by MD simulation using parameters for a protein in a liquid formulation. In some embodiments, oxidation of at least about 30% (such as at least about any of 35%, 40%, 45%, 50%, 55%, 60%, 65%, 70%, 75%, 80%, 85%, 90%, 95%, or more) of a residue in an oxidation assay indicates susceptibility to oxidation.

### VI. Administration of Protein Formulations

The liquid formulation is administered to a mammal in need of treatment with the protein (e.g., an antibody), preferably a human, in accord with known methods, such as intravenous administration as a bolus or by continuous infusion over a period of time, by intramuscular, intraperitoneal, intracerebrospinal, subcutaneous, intra-articular, intrasynovial, intrathecal, oral, topical, inhalation, or intravitreal routes. In one embodiment, the liquid formulation is administered to the mammal by intravenous administration. For such purposes, the formulation may be injected using a syringe or via an IV line, for example. In one embodiment, the liquid formulation is administered to the mammal by subcutaneous administration. In yet another embodiment, the liquid formulation is administered by intravitreal administration.

The appropriate dosage (“therapeutically effective amount”) of the protein will depend, for example, on the condition to be treated, the severity and course of the condition, whether the protein is administered for preventive or therapeutic purposes, previous therapy, the patient's clinical history and response to the protein, the type of protein used, and the discretion of the attending physician. The protein is suitably administered to the patient at one time or over a series of treatments and may be administered to the patient at any time from diagnosis onwards. The protein may be administered as the sole treatment or in conjunction with other drugs or therapies useful in treating the condition in question. As used herein the term “treatment” refers to both therapeutic treatment and prophylactic or preventative measures. Those in need of treatment include those already with the disorder as well as those in which the disorder is to be prevented. As used herein a “disorder” is any condition that would benefit from treatment including, but not limited to, chronic and acute disorders or diseases including those pathological conditions which predispose the mammal to the disorder in question.

In a pharmacological sense, in the context of the invention, a “therapeutically effective amount” of a protein (e.g., an antibody) refers to an amount effective in the prevention or treatment of a disorder for the treatment of which the antibody is effective. In some embodiments, the therapeutically effective amount of the protein administered will be in the range of about 0.1 to about 50 mg/kg (such as about 0.3 to about 20 mg/kg, or about 0.3 to about 15 mg/kg) of patient body weight whether by one or more administrations. In some embodiments, the therapeutically effective amount of the protein is administered as a daily dose, or as multiple daily doses. In some embodiments, the therapeutically effective amount of the protein is administered less frequently than daily, such as weekly or monthly. For example, a protein can be administered at a dose of about 100 to about 400 mg (such as about any of 100, 150, 200, 250, 300, 350, or 400 mg, including any ranges between these values) every one or more weeks (such as every 1, 2, 3, or 4 weeks or more, or every 1, 2, 3, 4, 5, or 6 months or more) or is administered a dose of about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 15.0, or 20.0 mg/kg every one or more weeks (such as every 1, 2, 3, or 4 weeks or more, or every 1, 2, 3, 4, 5, or 6 months or more). The dose may be administered as a single dose or as multiple doses (e.g., 2, 3, 4, or more doses), such as infusions. The progress of this therapy is easily monitored by conventional techniques.

### VII. Methods to Measure Degradation of NAT

The invention herein also provides a method of screening a liquid formulation for reduced oxidation of a protein. To effectively protect a protein in a formulation, the NAT in the formulation must be sacrificially oxidized over susceptible Trp residues; as such, NAT degradants can be expected to form during handling and storage of drug products containing NAT. Understanding the rate and degradation pathways for NAT is important as the degraded NAT species present in the drug product would be administered to the patient along with therapeutic protein. A single report on NAT degradation in the literature used a two-dimensional size exclusion chromatography trapping method, along with a multiple reaction monitoring LC-MS method, to identify and quantify two NAT degradants (N-Ac-PIC, 2b, and N-Ac-3a,8a-dihydroxy-PIC, 3b) observed in concentrated HSA solutions after long-term storage at elevated temperature (Fang, L., et al., J Chromatogr A, 2011, 1218(41):7316-24). Degradation of Trp itself has been more comprehensively studied (Ji, J. A., et al., J Pharm Sci, 2009, 98(12):4485-500; Simat, T. J. and H. Steinhart, J Agric Food Chem, 1998, 46(2):490-498) and a much larger group of degradants has been reported, including PIC (2a), oxyindolylalanine (Oia, 4a), dioxyindolylalanine (DiOia, 5a), kynurenine (Kyn, 6a), N-formyl-kynurenine (NFK, 7a), and 5-hydroxy-Trp (5-OH-Trp, 8a).

In some aspects, the invention provides method for measuring N-acetyl tryptophan (NAT) degradation in a composition comprising N-acetyl tryptophan, the method comprising a) applying the composition to a reverse phase chromatography material, wherein the composition is loaded onto the chromatography material equilibrated in a solution comprising a mobile phase A and a mobile phase B, wherein mobile phase A comprises acid in water and mobile phase B comprises acid in an organic solvent, b) eluting the composition from the reverse phase chromatography material with a solution comprising mobile phase A and mobile phase B wherein the ratio of mobile phase B to mobile phase A is increased compared to step a), wherein NAT degradants elute from the chromatography separately from intact NAT, c) quantifying the NAT degradants and the intact NAT. In embodiments, the ratio of mobile phase B to mobile phase A in step a) is about any of 1:99, 2:98, 3:97, 4:96, 5:95, 6:94, 7:93, 8:92, 9:91, or 10:90. In embodiments, the ratio of mobile phase B to mobile phase A in step a) is about 2:98. In some embodiments, the ratio of mobile phase B to mobile phase A in step b) increases linearly. In other embodiment, the ratio of mobile phase B to mobile phase A in step b) increases stepwise. In some embodiments, the organic solvent is acetonitrile.

In some aspects, the invention provides method for measuring N-acetyl tryptophan (NAT) degradation in a composition comprising N-acetyl tryptophan, the method comprising a) applying the composition to a reverse phase chromatography material, wherein the composition is loaded onto the chromatography material equilibrated in a solution comprising a mobile phase A and a mobile phase B, wherein mobile phase A comprises acid in water and mobile phase B comprises acid in acetonitrile, b) eluting the composition from the reverse phase chromatography material with a solution comprising mobile phase A and mobile phase B wherein the ratio of mobile phase B to mobile phase A is increased compared to step a), wherein NAT degradants elute from the chromatography separately from intact NAT, c) quantifying the NAT degradants and the intact NAT. In embodiments, the ratio of mobile phase B to mobile phase A in step a) is about any of 1:99, 2:98, 3:97, 4:96, 5:95, 6:94, 7:93, 8:92, 9:91, or 10:90. In embodiments, the ratio of mobile phase B to mobile phase A in step a) is about 2:98. In some embodiments, the ratio of mobile phase B to mobile phase A in step b) increases linearly. In other embodiment, the ratio of mobile phase B to mobile phase A in step b) increases stepwise.

In some embodiments, the flow rate of the chromatography is about any of 0.25 mL/minute, 0.5 mL/minute, 0.75 mL/minute, 1.0 mL/minute, 1.25 mL/minute, 1.5 mL/minute, 1.75 mL/minute, 2.0 mL/minute, or 2.5 mL/minute. In some embodiments, the flow rate of the chromatography is about any of 1.0 mL/min.

In some embodiments, the ratio of mobile phase B to mobile phase A is increased to about any of 25:75, 28:72, 30:70, 32:68, or 35:65. In some embodiments, the ratio of mobile phase B to mobile phase A is increased to about 30:70. In some embodiments, the ratio of mobile phase B to mobile phase A is increased to about any of 25:75, 28:72, 30:70, 32:68, or 35:65 in about any of 14, 15, 16, 17 or 18 minutes from the start of chromatography. In some embodiments, the ratio of mobile phase B to mobile phase A is increased to about any of 25:75, 28:72, 30:70, 32:68, or 35:65 in about 16 minutes from the start of chromatography. In some embodiments, the ratio of mobile phase B to mobile phase A is increased to about 30:70. In some embodiments, the ratio of mobile phase B to mobile phase A is increased to about 30:70 in about 16 minutes from the start of chromatography. In some embodiments, the flow rate is about 1 mL/min.

In some embodiments, the ratio of mobile phase B to mobile phase A is increased to about any of 85:15, 90:10, or 95:5. In some embodiments, the ratio of mobile phase B to mobile phase A is increased to about 30:70. In some embodiments, the ratio of mobile phase B to mobile phase A is increased to about any of 85:15, 90:10, or 95:5 in about any of 16, 17, 18, 19 or 20 minutes from the start of chromatography. In some embodiments, the ratio of mobile phase B to mobile phase A is increased to about any of 85:15, 90:10, or 95:5 in about 18.1 minutes from the start of chromatography. In some embodiments, the ratio of mobile phase B to mobile phase A is increased to about 90:10. In some embodiments, the ratio of mobile phase B to mobile phase A is increased to about 90:10 in about 18.1 minutes from the start of chromatography. In some embodiments, the flow rate is about 1 mL/min.

In some embodiments, the ratio of mobile phase B to mobile phase A is increased to about any of 24:76, 25:75, 26:70, 27:73, or 28:71. In some embodiments, the ratio of mobile phase B to mobile phase A is increased to about 26:74. In some embodiments, the ratio of mobile phase B to mobile phase A is increased to about any of 24:76, 25:75, 26:70, 27:73, or 28:71 in about any of 12, 13, 14, 15 or 16 minutes from the start of chromatography. In some embodiments, the ratio of mobile phase B to mobile phase A is increased to about any of 24:76, 25:75, 26:70, 27:73, or 28:71 in about 14 minutes from the start of chromatography. In some embodiments, the ratio of mobile phase B to mobile phase A is increased to about 26:74. In some embodiments, the ratio of mobile phase B to mobile phase A is increased to about 26:74 in about 14 minutes from the start of chromatography. In some embodiments, the flow rate is about 1 mL/min.

In some embodiments, the ratio of mobile phase B to mobile phase A is increased to about any of 85:15, 90:10, or 95:5. In some embodiments, the ratio of mobile phase B to mobile phase A is increased to about 30:70. In some embodiments, the ratio of mobile phase B to mobile phase A is increased to about any of 85:15, 90:10, or 95:5 in about any of 14.5, 15.5, 16.5, 17.5 or 18.5 minutes from the start of chromatography. In some embodiments, the ratio of mobile phase B to mobile phase A is increased to about any of 85:15, 90:10, or 95:5 in about 16.5 minutes from the start of chromatography. In some embodiments, the ratio of mobile phase B to mobile phase A is increased to about 90:10. In some embodiments, the ratio of mobile phase B to mobile phase A is increased to about 90:10 in about 16.5 minutes from the start of chromatography. In some embodiments, the flow rate is about 1 mL/min.

In some embodiments, mobile phase A comprises any of about 0.01%, 0.05%, 0.1%, 0.5%, or 1.0% (v/v) acid in water. In some embodiments, mobile phase A comprises about 0.1% acid in water. In some embodiments, the acid is formic acid. In some embodiments, mobile phase A comprises about 0.1% formic acid in water. In some embodiments, mobile phase 5 comprises any of about 0.01%, 0.05%, 0.1%, 0.5%, or 1.0% (v/v) acid in acetonitrile. In some embodiments, mobile phase B comprises about 0.1% acid in acetonitrile. In some embodiments, the acid is formic acid. In some embodiments, mobile phase B comprises about 0.1% formic acid in acetonitrile. In some embodiments, mobile phase A comprises about 0.1% formic acid in water and mobile phase B comprises about 0.1% formic acid in acetonitrile.

In some aspects, the invention provides method for measuring N-acetyl tryptophan (NAT) degradation in a composition comprising N-acetyl tryptophan, the method comprising a) applying the composition to a reverse phase chromatography material, wherein the composition is loaded onto the chromatography material equilibrated in a solution comprising a mobile phase A and a mobile phase B, wherein mobile phase A comprises 0.1% (v/v) formic acid in water and mobile phase B comprises 0.1% (v/v) formic acid in an organic solvent, at a ratio of mobile phase B to mobile phase A of 2:98; b) eluting the composition from the reverse phase chromatography material with a solution comprising mobile phase A and mobile phase B wherein the ratio of mobile phase B to mobile phase A is increased to about 70:30 and then to about 90:10, wherein NAT degradants elute from the chromatography separately from intact NAT, c) quantifying the NAT degradants and the intact NAT. In some embodiments, the flow rate is about 1.0 mL/minute and the ratio of mobile phase B to mobile phase A is increased to about 70:30 in about 16 minutes after the start of chromatography and then to about 90:10 after about 18 minutes from the start of chromatography. In some embodiments, the organic solvent is acetonitrile.

In some aspects, the invention provides method for measuring N-acetyl tryptophan (NAT) degradation in a composition comprising N-acetyl tryptophan, the method comprising a) applying the composition to a reverse phase chromatography material, wherein the composition is loaded onto the chromatography material equilibrated in a solution comprising a mobile phase A and a mobile phase B, wherein mobile phase A comprises 0.1% (v/v) formic acid in water and mobile phase B comprises 0.1% (v/v) formic acid in acetonitrile, at a ratio of mobile phase B to mobile phase A of 2:98; b) eluting the composition from the reverse phase chromatography material with a solution comprising mobile phase A and mobile phase B wherein the ratio of mobile phase B to mobile phase A is increased to about 70:30 and then to about 90:10, wherein NAT degradants elute from the chromatography separately from intact NAT, c) quantifying the NAT degradants and the intact NAT. In some embodiments, the flow rate is about 1.0 mL/minute and the ratio of mobile phase B to mobile phase A is increased to about 70:30 in about 16 minutes after the start of chromatography and then to about 90:10 after about 18 minutes from the start of chromatography.

In some aspects, the invention provides method for measuring N-acetyl tryptophan (NAT) degradation in a composition comprising N-acetyl tryptophan, the method comprising a) applying the composition to a reverse phase chromatography material, wherein the composition is loaded onto the chromatography material in a solution comprising a mobile phase A and a mobile phase B, wherein mobile phase A comprises 0.1% (v/v) formic acid in water and mobile phase B comprises 0.1% (v/v) formic acid in acetonitrile, at a ratio of mobile phase B to mobile phase A of 2:98; b) eluting the composition from the reverse phase chromatography material with a solution comprising mobile phase A and mobile phase B wherein the ratio of mobile phase B to mobile phase A is increased to about 74:26 and then to about 90:10, wherein NAT degradants elute from the chromatography separately from intact NAT, c) quantifying the NAT degradants and the intact NAT. In some embodiments, the flow rate is about 1.0 mL/minute and the ratio of mobile phase B to mobile phase A is increased to about 74:26 in about 14 minutes after the start of chromatography and then to about 90:10 after about 16.5 minutes from the start of chromatography.

In some embodiments, the reverse phase chromatography material comprises a C8 moiety or a C18 moiety. In some embodiments, the reverse phase chromatography material comprises a C18 moiety. In some embodiments, the reverse phase chromatography material comprises a solid support. In some embodiments, the solid support comprises silica. In some embodiments, the reverse phase chromatography material is contained in a column. In some embodiments, the reverse phase chromatography material is a high performance liquid chromatography (HPLC) material or an ultra-high performance liquid chromatography (UPLC) material. In some embodiments, the reverse phase chromatography column is an Agilent ZORBAX® SB-C18 chromatography column. In some embodiments, the reverse phase chromatography column is an Agilent ZORBAX® SB-C18 3.5 μm, 4.6×75 mm chromatography column.

In some embodiments, the chromatography is performed at a temperature ranging from about 0° C. to about 30° C. In some embodiments, the chromatography is performed at any of about 0° C., 5° C., 20° C., or 30° C. In some embodiments, the chromatography is performed at room temperature. In some embodiments, the chromatography is performed at about 5° C. In some embodiments, the chromatography is performed at 5° C.±3° C.

In some embodiments, NAT and NAT degradation products are detected by absorbance at 240 nm. In some embodiments, NAT degradation products are identified by mass spectrometry. In some embodiments, the concentration of NAT in the composition is about 10 nM to about 1 mM. In some embodiments, the concentration of NAT in the composition is less than about any of 10 nM, 25 nM, 50 nM, 75 nM, 100 nM, 250 nM, 500 nM, 750 nM, 1 μM, 2.5 μM, 5 μM, 7.5 μM, 10 μM, 25 μM, 50 μM, 75 μM, 100 μM, 250 μM, 500 μM, 750 μM, or 1 mM. In some embodiments, the concentration of NAT in the composition ranges is between about 10 nM and about 100 nM, about 100 nM and about 500 nM, about 500 nM and about 1, about 1 μM and about 100 about 100 μM and about 500 μM, or about 500 μM and about 1 mM.

In some embodiments of the above methods, the NAT degradation products include one or more of N-Ac-(H, 1,2,3,3a,8,8a-hexahydro-3a-hydroxypyrrolo [2,3-b]-indole 2-carboxylic acid) (N-Ac-PIC), N-Ac-oxyindolylalanine (N-Ac-Oia), N-Ac-N-formyl-kynurenine (N-Ac-NFK), N-Ac-kynurenine (N-Ac-Kyn) and N-Ac-2a,8a-dihydroxy-PIC.

In some embodiments the invention provides methods for measuring N-acetyl tryptophan (NAT) degradation in a composition comprising N-acetyl tryptophan and a polypeptide, the method comprising a) denaturing the polypeptide, b) removing the polypeptide from the composition, c) applying the composition to a reverse phase chromatography material, wherein the composition is loaded onto the chromatography material in a solution comprising a mobile phase A and a mobile phase B, wherein mobile phase A comprises acid in water and mobile phase B comprises acid in acetonitrile, d) eluting the composition from the reverse phase chromatography material with a solution comprising mobile phase A and mobile phase B wherein the ratio of mobile phase B to mobile phase A is increased compared to step a), wherein NAT degradants elute from the chromatography separately from intact NAT, e) quantifying the NAT degradants and the intact NAT. In some embodiments, the polypeptide is denatured by treatment with guanidine. In some embodiments, the polypeptide is denatured with guanidine wherein the guanidine is added to the composition to a final concentration of about 7 M to about 9 M. In some embodiments, the polypeptide is denatured with guanidine wherein the guanidine is added to the composition to a final concentration of about 8 M.

In some embodiments the invention provides methods for measuring N-acetyl tryptophan (NAT) degradation in a composition comprising N-acetyl tryptophan and a polypeptide, the method comprising a) diluting the composition with about 8 M guanidine, b) removing the polypeptide from the composition, c) applying the composition to a reverse phase chromatography material, wherein the composition is loaded onto the chromatography material in a solution comprising a mobile phase A and a mobile phase B, wherein mobile phase A comprises acid in water and mobile phase B comprises acid in acetonitrile, d) eluting the composition from the reverse phase chromatography material with a solution comprising mobile phase A and mobile phase B wherein the ratio of mobile phase B to mobile phase A is increased compared to step a), wherein NAT degradants elute from the chromatography separately from intact NAT, e) quantifying the NAT degradants and the intact NAT.

In some embodiments of the above-embodiments, the composition is diluted in about 8M guanidine such that the final concentration of NAT in the composition ranges from about 0.01 mM to about 0.5 mM. In some embodiments of the above-embodiments, the composition is diluted in about 8M guanidine such that the final concentration of NAT in the composition ranges from about 0.05 mM to about 0.2 mM. In some embodiments of the above-embodiments, the composition is diluted in about 8M guanidine such that the final concentration of NAT in the composition is less than about any of 0.05 mM, 0.06 mM, 0.07 mM, 0.08 mM, 0.09 mM, 0.10 mM, 0.12 mM, 0.14 mM, 0.16 mM, 0.18 mM or about 0.2 mM. In some embodiments, the composition is diluted in about 8M guanidine such that the final concentration of polypeptide in the composition is less than or equal to any of about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, or about 100 mg/mL. In some embodiments, the composition is diluted in about 8M guanidine such that the final concentration of polypeptide in the composition is about 5 mg/mL to about 10 mg/mL, about 10 mg/mL to about 15 mg/mL, about 15 mg/mL to about 20 mg/mL, about 20 mg/mL to about 25 mg/mL, about 25 mg/mL to about 30 mg/mL, about 30 mg/mL to about 35 mg/mL, about 35 mg/mL to about 40 mg/mL, about 40 mg/mL to about 45 mg/mL, about 145 mg/mL to about 50 mg/mL, or about 50 mg/mL to about 100 mg/mL.

In some embodiments the polypeptide is removed from the composition by filtration. In some embodiments the filtration uses a filtration membrane with a molecular weight cut-off of about 30 kDal.

In some embodiments of the above embodiments, the formulation has a pH of about 3.5 to about 7.0. In some embodiments of the above embodiments, the formulation has a pH of about 4.5 to about 7.0. In some embodiments, the formulation has a pH about any of 3.5, 4.0, 4.5, 5.0, 5.5, 6.0, 7.0, 7.5, or 8.0.

In some embodiments, the formulation further comprises one or more excipients selected from the group consisting of a stabilizer, a buffer, a surfactant, and a tonicity agent.

In some embodiments, the formulation is a pharmaceutical formulation suitable for administration to a subject. In some embodiments, the polypeptide is an antibody. In some embodiments, the antibody is a polyclonal antibody, a monoclonal antibody, a humanized antibody, a human antibody, a chimeric antibody, a multispecific antibody or antibody fragment.

In some embodiments, the invention provides a method to monitor degradation of NAT in a composition comprising measuring the degradation of NAT in a sample of the composition according to the methods describe above, wherein the method is repeated one or more times. In some embodiments, the method is repeated at least about any of two times, three times, four times, five times, six times, seven times, eight times, nine times, or ten times. In some embodiments the method is repeated daily, weekly, or monthly or any combination therein. In some embodiments, the method is repeated at least about every month, every two months, every three months, every four months, every five months, every six months, every nine months or at least about once a year.

In some embodiments, the invention provides a quality assay for a pharmaceutical composition, the quality assay comprising measuring degradation of NAT in a sample of the pharmaceutical composition according to the methods described above, wherein the amount of NAT degradants measured in the composition determines if the pharmaceutical composition is suitable for administration to an animal. In some embodiments, an amount of NAT degradants in the pharmaceutical composition of less than about any of 1 ppm, 2 ppm, 3 ppm, 4 ppm, 5 ppm, 6 ppm, 7 ppm, 8 ppm, 9 ppm, 10 ppm, 20 ppm, 30 ppm, 40 ppm, 50 ppm, 60 ppm, 70 ppm, 80 ppm, 90 ppm, or 100 ppm indicates that the pharmaceutical composition is suitable for administration to the animal. In some embodiments, an amount of NAT degradants in the pharmaceutical composition of less than about 10 ppm indicates that the pharmaceutical composition is suitable for administration to the animal.

### VIII. Articles of Manufacture

In another embodiment of the invention, an article of manufacture is provided comprising a container which holds the liquid formulation of the invention and optionally provides instructions for its use. In some embodiments, the liquid formulation comprises a protein (e.g. an antibody) and N-acetyl-tryptophan (NAT), wherein the protein comprises at least one tryptophan residue a) with a SASA of greater than about 50 Å² to about 250 Å² (such as greater than about any of 50, 60, 70, 80, 90, 100, 120, 140, 160, 180, 200, 225, or 250 Å², including any ranges between these values); b) with a SASA greater than about 15% to about 45% (such as greater than about any of 15, 20, 25, 30, 35, 40, or 45%); or c) predicted to be susceptible to oxidation by a machine learning algorithm trained on associations of tryptophan residue oxidation susceptibility with a plurality of molecule descriptors of the tryptophan residue based on MD simulations. In some embodiments, the amount of NAT in the liquid formulation is from about 0.1 mM to about 10 mM (such as about any of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM, including any ranges between these values). In some embodiments, the oxidation of the protein is reduced by about 40% to about 100% (such as by about any of 40%, 45%, 50%, 55%, 60%, 65%, 70%, 75%, 80%, 85%, 90%, 95%, 96%, 97%, 98%, 99%, or 100%, including any ranges between these values). In some embodiments, the liquid formulation is stable at about 0° C. to about 5° C. (such as about any of 0, 1, 2, 3, 4 or 5° C., including any ranges between these values) for at least about 12 months (such as at least about any of 12, 15, 18, 21, 24, 27, 30, 33, or 36 months, including any ranges between these values). In some embodiments, the concentration of the protein in the liquid formulation is from about 1 mg/mL to about 250 mg/mL. In some embodiments, the protein is a therapeutic protein. In some embodiments, the protein is an antibody. In some embodiments, the antibody is a polyclonal antibody, a monoclonal antibody, a humanized antibody, a human antibody, a chimeric antibody, a multispecific antibody or an antibody fragment. In some embodiments, the antibody is derived from an IgG1 antibody sequence. In some embodiments, the liquid formulation further comprises one or more excipients selected from the group consisting of a stabilizer, a buffer, a surfactant, and a tonicity agent. In some embodiments, the liquid formulation has a pH of about 4.5 to about 7.0. In some embodiments, the liquid formulation is an aqueous formulation.

Suitable containers include, for example, bottles, vials and syringes. The container may be formed from a variety of materials such as glass or plastic. An exemplary container is a 2-20 cc single use glass vial. Alternatively, for a multidose formulation, the container may be a 2-100 cc glass vial. The container holds the formulation and the label on, or associated with, the container may indicate directions for use. The article of manufacture may further include other materials desirable from a commercial and user standpoint, including other buffers, diluents, filters, needles, syringes, and package inserts with instructions for use.

Package insert refers to instructions customarily included in commercial packages of therapeutic products that contain information about the indications, usage, dosage, administration, contraindications and/or warnings concerning the use of such therapeutic products.

Kits are also provided that are useful for various purposes, e.g., for reducing oxidation of a protein in a liquid formulation or for screening a liquid formulation for reduced oxidation of a protein, optionally in combination with the articles of manufacture. Kits of the invention include one or more containers comprising a protein (e.g. an antibody) comprising at least one tryptophan residue a) with a SASA of greater than about 50 Å² to about 250 Å² (such as greater than about any of 50, 60, 70, 80, 90, 100, 120, 140, 160, 180, 200, 225, or 250 Å², including any ranges between these values); b) with a SASA greater than about 15% to about 45% (such as greater than about any of 15, 20, 25, 30, 35, 40, or 45%); or c) predicted to be susceptible to oxidation by a machine learning algorithm trained on associations of tryptophan residue oxidation susceptibility with a plurality of molecule descriptors of the tryptophan residue based on MD simulations; NAT, AAPH, and/or instructions for use in accordance with any of the methods described herein. Instructions supplied in the kits of the invention are typically written instructions on a label or package insert (e.g., a paper sheet included in the kit), but machine-readable instructions (e.g., instructions carried on a magnetic or optical storage disk) are also acceptable.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1. Assessment of NAT Protection from Oxidation

### SASA Calculation

SASA for the indicated protein residues was calculated using the in silico all-atom molecular dynamics modeling method described in Sharma, V. et al. (supra). Briefly, the structure of the protein was obtained from either the 3D crystal structure or a homology model, adding ions and explicit solvent molecules as needed. The SASA was calculated using g_sas of GROMACS, with mutual information calculation implemented locally (Eisenhaber F. et al., J. Comput. Chem. 16(3):273-284, 1995; Lange O. F. et al. Proteins 70(4):1294-1312, 2008). The root mean square fluctuations, the hydrogen bonds, and the secondary structure were calculated using statusg_rsmf, g_hbond, and dssp of GROMACS, respectively. Shannon entropy and mutual information were calculated using previously published methods (Kortkhonjia E, et al., MAbs 5(2):306-322, 2013). MD simulations were conducted using Amber 11 (FF99SB fixed-charge force field; SASA was calculated using areaimol (Bailey S, Acta. Crystallogr. D. Biol. Crystallogr. 50(Pt 5):760-763, 1994); 100-ns trajectories were used as they provided sufficient data within available computational power.

### AAPH Stress

Proteins Mab1, Mab2, and Mab3/Mab4 were dialyzed into a sodium acetate buffer (20 mM sodium acetate pH 5.5) and Mab5/Mab6 was dialyzed into a histidine-based buffer (20 mM histidine hydrochloride pH 5.5). Protein solutions were diluted to a final concentration of 1 mg/mL protein in the corresponding buffer and 1 mM 2,2′-Azobis(2-amidinopropane) dihydrochloride (AAPH) was added. N-acetyl-Trp (NAT) was added from a concentrated stock solution to each protein solution at concentrations from 0 to 5 mM. Samples were incubated at 40° C. for 16 hours followed by quenching with methionine and buffer exchange to the initial dialysis buffer plus 100 mM sucrose.

### LC-MS Tryptic Peptide Mapping

Site-specific modifications of AAPH-stressed samples of Mab2, Mab1, and Mab3/Mab4 were monitored using a microscale tryptic peptide digest followed by liquid chromatography-mass spectrometry (LC-MS) (Anderson, N. et al., Nov. 20, 2014, American Pharmaceutical Review). 30 μL (250 μg) of each stressed sample was diluted with 190 μL of reduction carboxymethylation buffer (6M guanidine HCL, 360 mM Tris, 2 mM EDTA, pH 8.6) to denature the protein. Following denaturation, 4 μL of 1M DTT was added to each mixture and the reduction reactions were incubated at 37° C. for 1 hour. The samples were then carboxymethylated by the addition of 10.4 μL of iodoacetic acid and stored in the dark at room temperature for 15 minutes. The alkylation reactions were quenched by the addition of 2 μL of 1M DTT. The reduced and alkylated samples were buffer exchanged on PD-10 columns (GE Healthcare) into trypsin digestion buffer (25 mM Tris, 2 mM CaCl₂, pH 8.2). The samples were then digested by adding sequencing grade trypsin (Promega) at an enzyme to protein ratio of 1:50 by weight. The digestion reactions were incubated at 37° C. for 4 hours and then quenched by adding 100% formic acid (FA) to the sample to a final FA concentration of 3.0% (v/v).

Peptide mapping of each digested sample was performed on a Waters Acquity H-Class UHPLC coupled to a Thermo Q Exactive Plus high resolution mass spectrometry system (HRMS). Separation of 10 μg injections of the digested samples was performed on a CSH C18 column (Waters, 1.7 μm particle size, 2.1 mm×150 mm) running at a flow rate of 0.3 mL/min and with column temperature controlled at 77° C. Solvent A consisted of 0.1% FA in water and Solvent B consisted of 0.1% FA in acetonitrile. The gradient is shown in Table 1. Column effluent was monitored at 214 nm. Full MS-SIM data were collected at a resolution of 17,500 over a scan range of 200-2000 m/z. Electron spray ionization in positive ion mode was achieved by using a needle spray voltage of 3.50 kV. Oxidation-prone sites of interest were previously characterized for each mAb using the same microscale tryptic digest followed by LC/MS-MS with MS/MS fragmentation used for residue-specific localization of the PTM. The oxidation level at each site was determined by extracted ion chromatography (EIC) using the Thermo XCalibur biopharmaceutical characterization software. The relative percentage of oxidation was calculated by dividing the peak area of the oxidized peptide species by the sum of the peak area of the native and oxidized peptides. Total oxidation values reported for tryptophan sites are the sum of Wₒₓ₁ (+16) and NFK/Wₒₓ₂ (+32) only. For more details on peptide mapping see Andersen, N. et al., Rapid UHPLC-HRMS Peptide Mapping for Monoclonal Antibodies. Amer. Pharm. Rev., 2014.

TABLE 1

| Time (min) | % Solvent A | % Solvent B |
|---|---|---|
| 0.0 | 99 | 1 |
| 2.0 | 87 | 13 |
| 9.5 | 62 | 38 |
| 12.5 | 25 | 75 |
| 12.6 | 10 | 90 |
| 13.0 | 10 | 90 |
| 13.1 | 99 | 1 |
| 22.0 | 99 | 1 |

NAT Protection from Oxidation

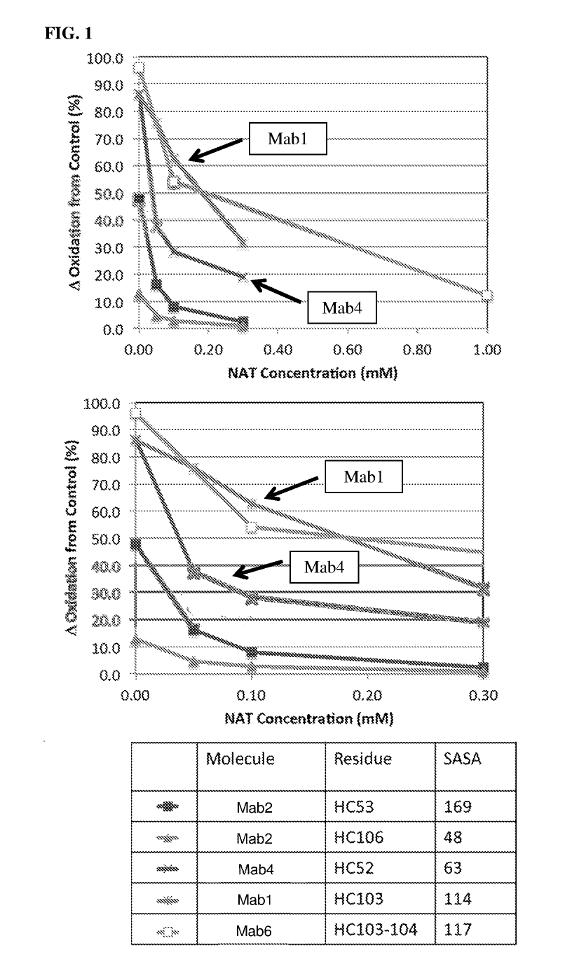

The following oxidation-prone tryptophan residues were assessed for NAT protection from AAPH-induced oxidation: Mab2 W53 and W106; Mab4 W52; Mab1 W103; and Mab6 W103/104. Each protein was subjected to AAPH stress as described above using 1 mM AAPH. NAT was added at 0, 0.05, 0.1, and 0.3 mM for Mab2, Mab4, and Mab1, and at 0, 0.1, and 1 mM for Mab6. As shown in FIG. 1 and Tables 2 and 3, NAT was able to protect each tested residue from oxidation resulting from AAPH stress.

TABLE 2
Oxidation of tryptophan residues

| | | | No AAPH | AAPH | | | | | |
|---|---|---|---|---|---|---|---|---|---|
| | | | NAT (mM) | | | | | | |
| Protein | Residue | SASA | 0 | 0 | 0.05 | 0.1 | 0.3 | 1 | 5 |
| Mab2 | W53 | 169 | 0.6 | 38 | 17.1 | 8.6 | 3.1 | n/a | n/a |
| Mab2 | W106 | 48 | 0.2 | 6.3 | 4.8 | 2.9 | 1 | n/a | n/a |
| Mab4 | W52 | 63 | 0.3 | 50 | 37.9 | 28.6 | 19.3 | n/a | n/a |
| Mab1 | W103 | 114 | 3.2 | 80 | 79.7 | 66.7 | 35.3 | n/a | n/a |
| Mab6 | W103/104 | 117 | 0.4 | 96.6 | n/a | 54 | n/a | 12.5 | n/a |

TABLE 3
Protection of protein

| Protein | Residue | Protection at 0.1 mM NAT (%)¹ |
|---|---|---|
| Mab2 | W53 | 79 |
| Mab2 | W106 | 56 |
| Mab4 | W52 | 43 |
| Mab1 | W103 | 17 |
| Mab6 | W103/104 | 44 |

¹Calculated as (ΔOxidation at 0 mM NAT − ΔOxidation at 0.1 mM NAT)/ΔOxidation at 0 mM NAT

### Prediction of Tryptophan Oxidation Susceptibility

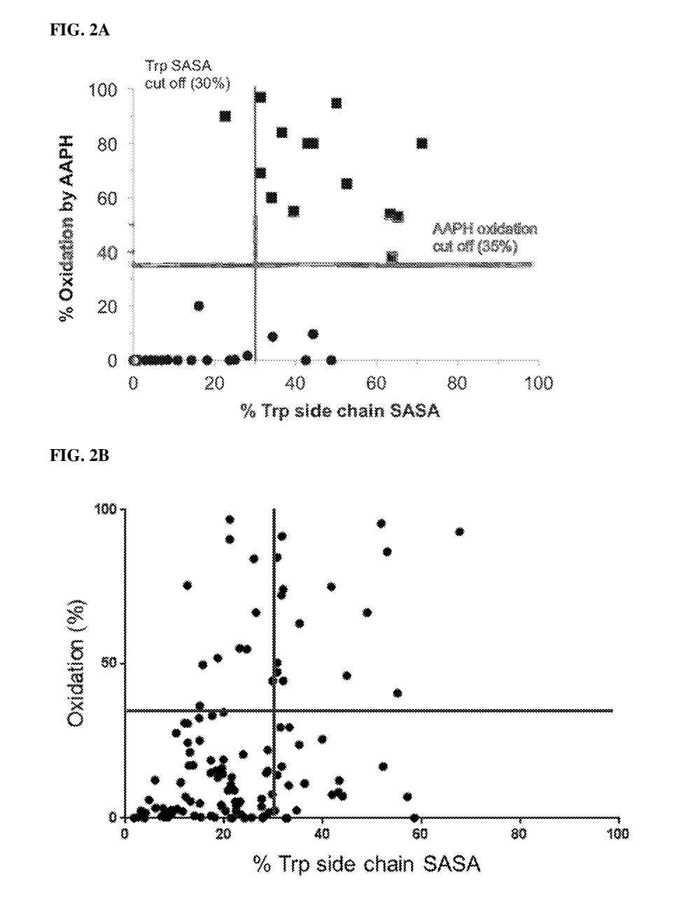

As shown in FIG. 2A, the % oxidation by AAPH as a function of tryptophan residue SASA was plotted using data from 38 IgG1 mAbs. Using a cutoff of 30% SASA, 87% of the examined residues had oxidation levels greater than 35%. A single molecule descriptor of tryptophan residues, % SASA, was highly accurate in predicting susceptibility to oxidation in this population of antibodies. However, as shown in FIG. 2B, expanding the data set to include tryptophan residues from 121 mAbs with diverse frameworks (e.g., IgG1, IgG2, IgG4, murine) resulted in less accurate prediction of oxidation susceptibility based solely on % SASA.

### Example 2. Machine Learning for Prediction of Tryptophan Oxidation Susceptibility

Using a single simulation-based molecule descriptor, such as SASA, can yield highly accurate predictions for tryptophan oxidation susceptibility in certain conditions, such as for specific IgG subclasses. However, when predicting tryptophan oxidation susceptibility of residues across diverse frameworks, accuracy can be improved by using multiple molecule descriptors. We used machine learning to correlate a set of MD simulation-based molecule descriptors with tryptophan oxidation susceptibility, resulting in a model that can be used to accurately predict stability of test tryptophan residues for which no experimental data on oxidation susceptibility is available, allowing for a quicker pipeline for selecting candidate molecules. Furthermore, the relative importance of the molecule descriptors in the model was determined, potentially pointing to underlying mechanisms that drive stability.

### Molecule Descriptors

The following molecule descriptors were calculated using the in silico all-atom molecular dynamics modeling method described above. Six MD simulations were run for each tryptophan residue using the following parameters: Fv-region only, 100 ns snapshot per simulation, explicit water, constant pressure, 3 simulations with protonated HIS, 3 simulations with deprotonated HIS (“pH”), and 3 fs step size. MD-derived molecule descriptors included circular fingerprinting of local chemical environment: charge, hydrophobicity; hydrogen bonding; and local structure of backbone and amino acid sidechains.

Number of Aspartic Acid Sidechain Oxygens within 7 Å of Tryptophan Delta Carbon

For each frame of each molecule simulation, all atoms within 7 Å of the delta carbon of each tryptophan were tracked. Of these atoms, those that were oxygen atoms on the sidechain of any aspartic acid residue were counted. The final value represents the time-average of this count over the duration of the simulation.

### Sidechain SASA (Stdev)

For each frame of each molecule simulation, the solvent-accessible surface area (SASA) of each tryptophan sidechain was computed. Briefly, points of a sphere centered on each atom in the simulation were generated by adding together each atomic radius with the radius of a water molecule. All points that were within the radii of neighboring spheres were eliminated, and the areas between all of the remaining points were summed to produce a value for SASA. The final value of this descriptor represents the standard deviation of the SASA of the tryptophan sidechain atoms over the duration of the simulation.

### Delta Carbon SASA (Stdev)

For each frame of each simulation, the solvent-accessible surface area (SASA) of each tryptophan delta carbon was computed as described previously. The value of this descriptor represents the standard deviation of the SASA of the tryptophan delta carbon over the duration of the simulation.

Total Positive Charge within 7 Å of Tryptophan Delta Carbon (Stdev)

For each frame of each simulation, all atoms associated with a charged amino acid sidechain within 7 Å of the delta carbon of each tryptophan were tracked. The total positive charge of these atoms was added together. The final value represents the standard deviation of this quantity over the duration of the simulation.

### Backbone SASA (Stdev)

For each frame of each molecule simulation, the solvent-accessible surface area (SASA) of the backbone nitrogen atom of each tryptophan was computed. This descriptor is the standard deviation of the SASA of the backbone nitrogen atom over the duration of the simulation.

### Tryptophan Sidechain Angles

The chi1 and chi2 angle of the tryptophan sidechains was tracked through the simulation. When all of the chi1 and chi2 angles over many different tryptophan residues and simulations were graphed, clusters of commonly occurring angle combinations became apparent. K-means clustering was used to define the center of each of the 12 regions.

The angle region that was most predictive of tryptophan oxidation was “cluster 5” centered at Chi1=76 degrees and Chi2=98 degrees. For each individual tryptophan residue, the percentage of the time that it spent in cluster 5 was tracked over the simulation and was added to the random decision forest as a descriptor.

Packing Density within 7 Å of Tryptophan Delta Carbon

Packing density was calculated as the time-averaged number of protein atoms within a sphere of radius 7 Å centered on the tryptophan delta carbon.

### Tryptophan Backbone Angles (Stdev)

This descriptor was calculated by measuring the standard deviation of the psi angle associated with the backbone of each tryptophan residue over the duration of the simulation.

### Occupied Volume of Pseudo-Pi Orbitals

The sidechain of each tryptophan residue was treated as the base of a cylinder with a height of 9 Å to approximate the space occupied by tryptophan pi-orbitals. All atoms that fell within the volume of the cylinder during the simulation were tracked. The total volume of all of the protein atoms falling within the volume of the cylinder was calculated for each frame of the simulations. The final value represents the time-averaged volume of the tryptophan pi-orbitals that was occupied by other protein atoms.

### Backbone Flexibility

The root mean squared fluctuation of the backbone nitrogen of each tryptophan residue was calculated over each simulation. Briefly, each frame in the simulation was aligned. The distance traveled by each nitrogen atom was calculated for each frame. This distance for each frame was squared, and the average of this squared distance across all frames was taken. Finally, the square root was taken of this average of the squared distance to produce the root mean squared fluctuation of the backbone nitrogen of the tryptophan.

Total Negative Charge within 7 Å of Tryptophan Delta Carbon

For each frame of each simulation, all atoms associated with a charged amino acid sidechain within 7 Å of the delta carbon of each tryptophan were tracked. The total negative charge of these atoms was added together. The final value represents the time-average of this quantity over the duration of the simulation.

### Generation of Random Decision Forest

Values for a set of molecule descriptors for 121 tryptophan residues in 68 molecules with experimentally determined oxidation levels were calculated as described above. Tryptophan residues having greater than 35% oxidation were classified as “unstable”, while those having less than 35% oxidation were classified as “stable”. General molecule descriptors were also associated with each of the tryptophan residues, including IgG type, IgG framework information, CDR location of tryptophan residue, CDR length, previous and subsequent residues in sequence, and number of other oxidation hotspots.

Combinations of experimental data (stable or unstable tryptophan residue) and tryptophan molecule descriptors (simulation data) were used to train the random decision forest to learn which simulation-based outputs correspond to tryptophan stability. The accuracy of the random decision forest was evaluated over a range of parameters to identify optimal conditions for training, and the most important descriptors for predicting tryptophan oxidation were ranked using the random decision forest generated using optimized parameters.

Accuracy of the random decision forest was evaluated using two methods. In one method, “out of bag” error was calculated. Out of bag error has been proven in machine learning literature to be a reliable estimate of predictive model accuracy (James, G., et al., An introduction to Statistical Learning. Springer. pp 316-321, 2013). Briefly, bootstrap aggregating was applied to the training set X=x₁, . . . , xₙ, and the mean prediction error on each training sample xᵢ, using only the trees that did not have xᵢ in their bootstrap sample, was calculated. In the other method, the data set was split into a training set (80% of the data) used to train the random decision forest and a test set (the remaining 20% of the data) applied to the resulting random decision forest. The prediction error for the test set was used to calculate the model accuracy.

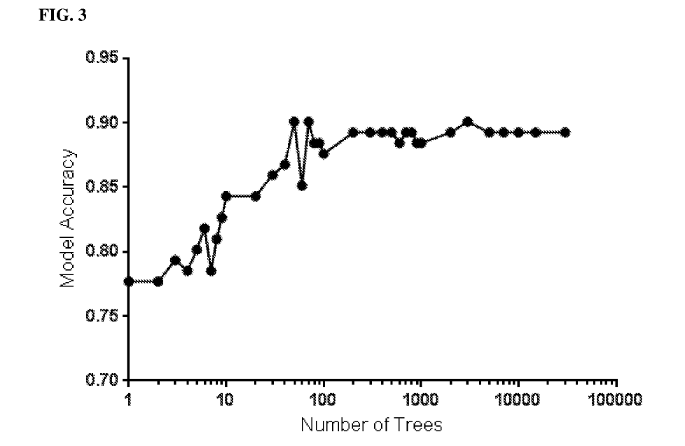

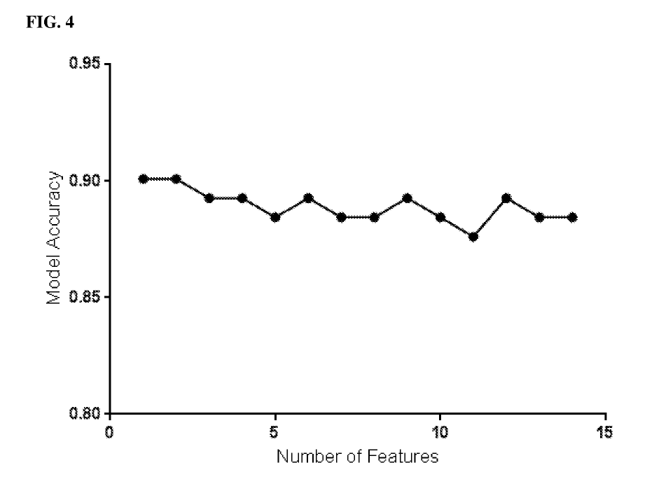

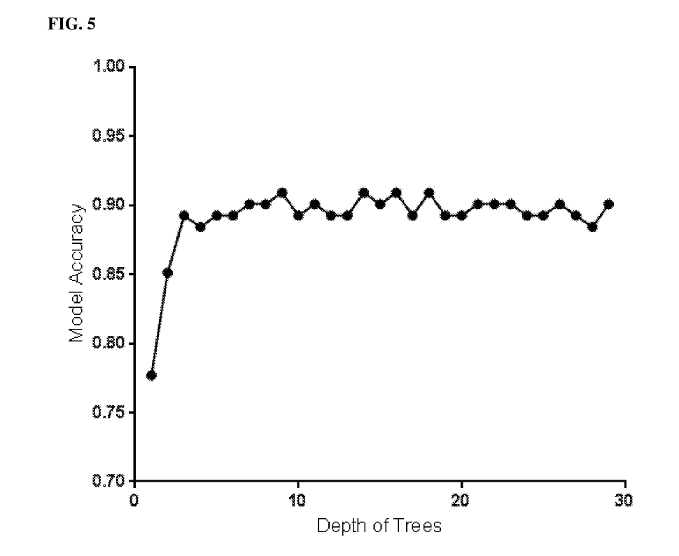

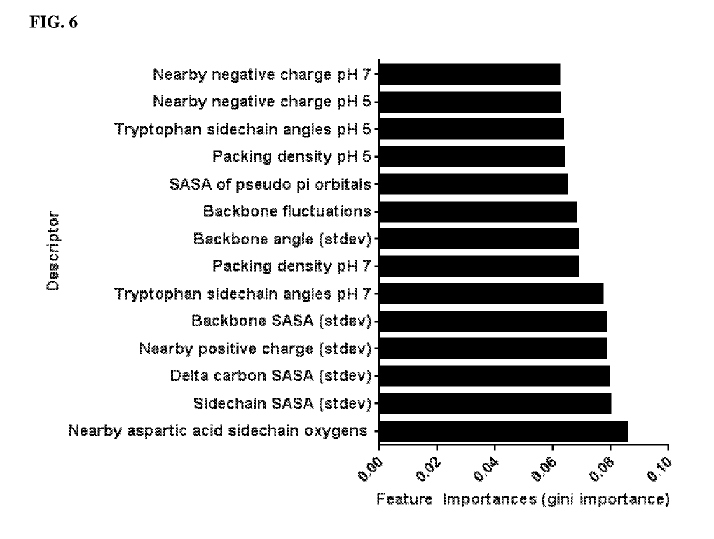

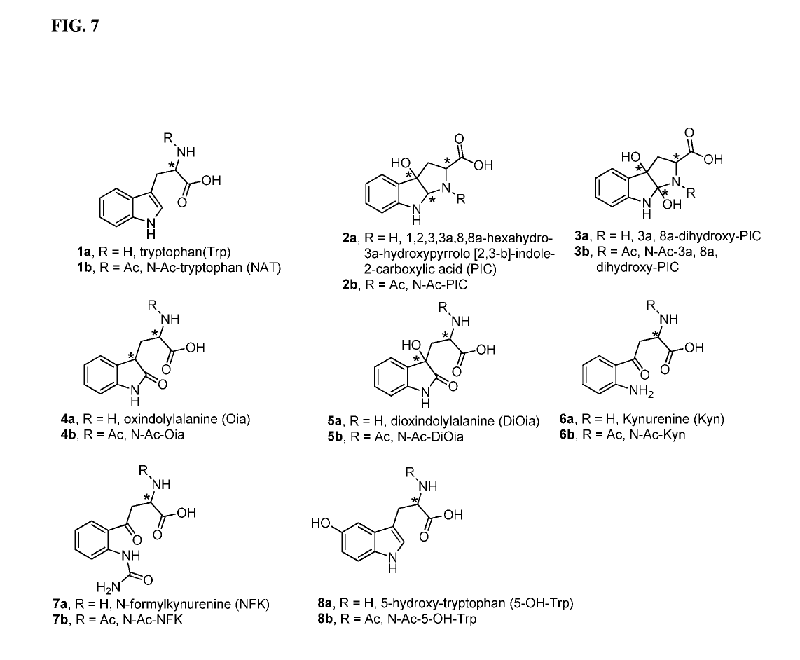

In order to determine optimal training conditions for the random decision forest, the following parameters were varied and model accuracy was evaluated: the number of individual decision trees included in the random decision forest (or estimators), the number of variables randomly selected for consideration at each branch of each tree in the random decision forest (or features), and the maximum number of times the pool of observations was divided into sub-branches (or tree depth). The optimal number of estimators for tryptophan oxidation model accuracy was greater than or equal to 200 (see FIG. 3). Accuracy above 85% was still achieved with as few as 30 estimators. The optimal number of features ranged between 1 and 4 (see FIG. 4). The optimal tree depth was greater than or equal to 5 (see FIG. 5). An accurate model was still achieved with a tree depth as low as 2. Based on these results, the following optimized parameters were used to generate a random decision forest: 5000 estimators, 3 features considered per node, and a tree depth of 10. The out of bag error calculation for the resulting optimized random decision forest yielded an accuracy of 89.2%. Splitting the data into training and test sets, the accuracy of the optimized random decision forest was found to be 88%, with 80% sensitivity and 89% specificity (see Table 4).

TABLE 4
Random decision forest accuracy

| | Predicted to be Stable | Predicted to be Unstable |
|---|---|---|
| Stable | 17 | 2 |
| Unstable | 1 | 4 |

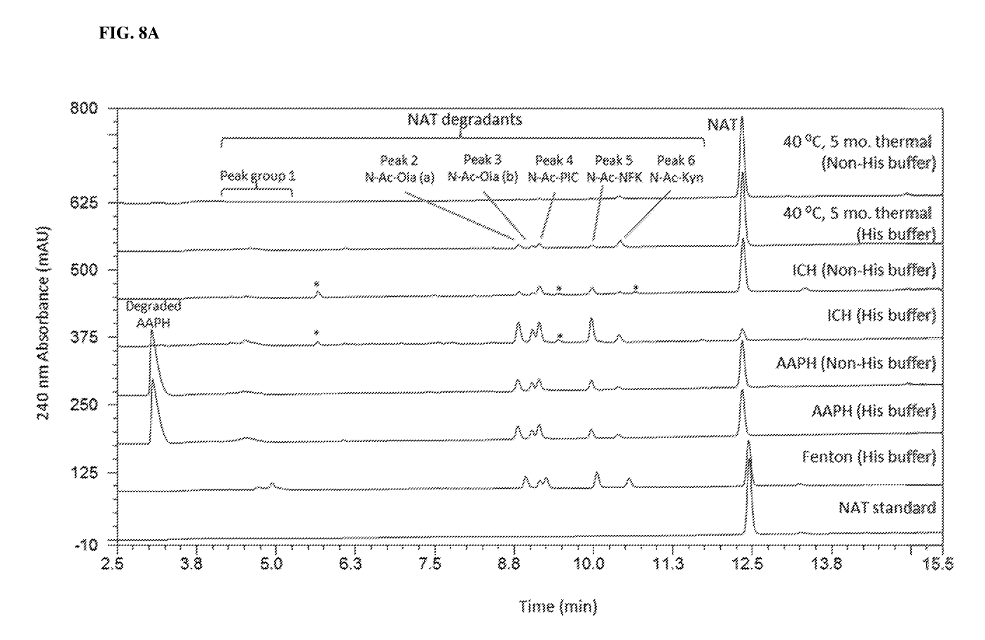

The relative importance of the simulation-based molecule descriptors in the optimized random decision forest was assessed using gini importance, and is shown for the top 14 molecule descriptors in FIG. 6. The most important descriptor was nearby aspartic acid sidechain oxygens, followed, in rank order, by sidechain SASA (stdev), delta carbon SASA (stdev), nearby positive charge (stdev), backbone SASA (stdev), tryptophan sidechain angles at pH7, packing density at pH 7, backbone angle (stdev), backbone fluctuations, SASA of pseudo pi orbitals, packing density at pH 5, tryptophan sidechain angles at pH 5, nearby negative charge at pH 5, and nearby negative charge at pH 7.

### Example 3. Characterization of NAT Degradation Under Different Stress Conditions and Formulations

To systematically assess NAT stability, we developed a reverse phase (RP) chromatography method combined with UV detection to quantitate NAT degradation. NAT was added to buffer systems typical of protein formulations and subjected to stresses designed to mimic those that recombinant proteins may be subjected to during typical manufacturing and storage conditions: alkyl peroxides, Fenton chemistry, UV light, and thermal stress (Grewal, P., et al., Mol Pharm, 2014. 11(4):1259-72; Ji, J. A., et al., J Pharm Sci, 2009. 98(12):4485-500; Torosantucci, R., et al., Pharm Res, 2014. 31(3): 541-53). In our studies, over 10 different NAT degradants were observed and the major species were identified.

### Chemicals

Except where noted, chemicals were purchased from Sigma. All chemicals used were of analytical purity grade. Protein therapeutic samples were produced in Chinese hamster ovary cells or E. coli and purified by a series of chromatography steps including affinity chromatography and/or ion-exchange chromatography. The synthesis of major NAT degradants (see FIG. 7 for NAT degradant structures) was accomplished by adapting literature methods as described below.

### General Synthetic Procedures

Anhydrous solvents were used where possible. Preparative reversed phase chromatography was performed on a Waters 2525 HPLC system using a Phenomenex Gemini-NX 10μ C18 110 Å 100 mm×30 mm preparative HPLC column. Mobile phase A=Milli-Q H₂O, 0.1% formic acid. Mobile phase B=acetonitrile, gradient=0-20% B from 0-12 min, fraction collection was triggered by UV signal threshold (10⁻¹ Au) at 254 nm. Fractions were analyzed by LC/MS for the presence of the desired product. For all preparative separations, the fractions containing the fronting and tailing portions of desired peaks were not included with the pooled fractions in order to improve purity.

LC/MS sample analysis of the final products was conducted using a Waters H-Class UPLC and the chromatography conditions described in the main text, in tandem with a Thermo Scientific Orbitrap Mass spectrometer. Full scan accurate mass data were collected at a resolution of 15,000 in positive ion mode over a scan range of 50-800 m/z. MS2 was performed on the top three ions with dynamic exclusion disabled.

NMR analysis was performed in perdeuterated DMF.

### General Procedure for Acetylation of Tryptophan Derivatives

The tryptophan derivatives were added to acetonitrile (anhydrous, J. T. Baker) (final concentration 200 mM). Di-isopropylethylamine (DIPEA, 5 eq) was added, followed immediately by 1.1 eq of acetic anhydride (Ac₂O). The reaction was stirred at room temp for 16 h. The mixture was filtered to remove unreacted starting material, and the solvent removed in vacuo. The material was dissolved in dimethylformamide (DMF) and the desired product was purified by prep-RPLC.

### N-Ac-Kyn (N-Ac-DL-Kynurenine) 6b

DL-Kynurenine (Sigma Aldrich) was acetylated by the general procedure above. DL-Kynurenine (800 mg, 3.84 mmol) was added to 40 ml of acetonitrile to form a light yellow suspension. DIPEA (5 eq) and Ac₂O (1.1 eq) were added. After stirring at room temp for 16 h, a majority of the suspended solid was dissolved and the solution was dark yellow/orange in color. Separation by preparative chromatography, and lyophilization of a portion of the isolated fraction material yielded 428 mg of a fluffy pale yellow solid. The obtained product (purity of 99% by RP-UPLC, 44% yield) was characterized by LC-MS (m/z=251.103) and NMR.

TLC analysis by UV and ninhydrin staining confirmed the absence of starting material.

N-Ac-NFK (Nα-acetyl-N′-formyl-kynurenine) 7b

N-Ac-NFK was synthesized by adapting the literature protocol reported by C. E. Dalgliesh in J. Chem. Soc. 1952, 137-141. A mixture of formic acid (Sigma, 98-100%, 360 μl) and acetic anhydride (J. T. Baker, Anhydrous 99%, 105 μl) was stirred for 30 min after which 100 mg of N-acetyl-DL-kynurenine was added. After reaction for 2 hr, LC/MS analysis still showed presence of starting material, at which point a second addition of formic acid (120 μl) and acetic anhydride (35 μl) was made to force the reaction to completion. LC/MS analysis performed 1 hr following the 2^{nd} addition showed absence of starting material and formation of desired product. The reaction mixture was added to 15 ml of water at room temperature and refrigerated. Pale brown crystals formed overnight. These were filtered, washed with ice-cold water, and the wet residue was lyophilized to yield 10 mg of the desired product as a fluffy pale brown solid. The obtained product (purity of 90% by RP-UPLC, 9.0% yield) was characterized by LC-MS (m/z=279.098) and NMR.

### Oia (Oxindoyl-DL-Alanine) Diastereomers 4a

Oia was synthesized by adapting the literature protocol reported by Itakura, K.; Uchida, K.; Kawakishi, S. in Chem. Res. Toxicol. 1994, 7, 185-190. DMSO (900 μl) and phenol (100 mg, 1.06 mmol) were premixed with 5 mL of 37% HCl at ambient temperature. DL-Trp (1.0 g, 4.9 mmol) was suspended in 30 ml of glacial acetic and added to the mixture. The reaction was stirred at ambient temperature. Progress was checked periodically by LC-MS. After 4 hours, LC/MS confirmed the loss of starting material and formation of two closely eluting peaks with the desired product mass (m/z=221). Removal of solvent under vacuum resulted in a dark brown syrup. The substance was dissolved in 4 ml of DMF. No attempt was made to separate the diastereomers; purification of the desired diastereomeric products were conducted using preparative-RPLC. Desired fractions were combined and lyophilized to produce 305 mg of a fluffy white solid. The obtained products (28% yield) was characterized by LC-MS (m/z=221).

### N-Ac-Oia (N-Acetyl-Ox-Indoyl Alanine) Diastereomers 4b

Ox-indoyl-DL-alanine diastereomers 4a (100 mg) was acetylated according to the general procedure above. After 16 hr, LC/MS confirmed that the reaction was complete. Solvent was removed in vacuo. Preparative chromatography and lyophilization yielded 56 mg of a fluffy white powder. No attempt was made to separate the diastereomers. The obtained diastereomeric product (purity of 89% by RP-UPLC, 47% yield) was characterized by LC-MS (m/z=263.102) and NMR. TLC analysis by UV and ninhydrin staining confirmed the absence of starting material.

### N-Ac-5-HTP (N-Acetyl-5-Hydroxy-Tryptophan) 8b

5-HTP 8a (150 mg) was acetylated according to the general procedure above. After 16 hr, LC/MS confirmed that the reaction was complete. Solvent was removed in vacuo. Preparative chromatography and lyophilization yielded 58 mg of a fluffy white powder. The obtained product (32% yield) was characterized by LC-MS (m/z=263.1) and NMR. TLC analysis by UV and ninhydrin staining confirmed the absence of starting material.

2,2′-Azobis(2-amidinopropane) dihydrochloride (AAPH) Oxidation Stress

AAPH (Calbio Chem, 99.8%) was used to model oxidative degradation by alkyl peroxides. Histidine and non-histidine buffers at pH 5.5 containing 0.3 mM NAT±5 mM L-Met pH 5.5 were treated with an aqueous AAPH solution to a final concentration of 1.0 mM AAPH. An equivalent volume of Milli-Q H₂O was added to control samples. Samples were incubated at 40° C. for 16 hours. Oxidation was quenched by the addition of L-Met to a final concentration of 20 mM. After the addition of the quenching solution, the final NAT concentration was 0.2 mM.

### Fenton Stress

FeCl₂ (Sigma Aldrich, 98% purity) and H₂O₂ (Sigma Aldrich, 30% w/w in H₂O) were added to a final concentration of 0.2 mM and 10 ppm, respectively, to a histidine-containing buffer at pH 5.5 with 0.3 mM NAT±5 mM L-Met, pH 5.5. Upon addition of H₂O₂ the vials were vortexed briefly and incubated for 3 hours at 40° C. Oxidation was quenched by the addition of L-Met to a final concentration of 100 mM. After the addition of the quenching solution, the final NAT concentration was 0.2 mM.

### Light Stress

A light box [Atlas SunTEST CPS+Xenon Light Box (Chicago, Ill.)] designed to conduct the drug substance/product photostability test recommended by the International Conference on Harmonization (ICH) Expert Working Group was utilized to provide light stress to NAT-containing samples. The ICH photostability test is defined as 1.2 million lux-hours of white light and 200 W-hours/m² of UV light; the light box was programmed to provide the stress over a period of 24 hours. Histidine and non-histidine buffers containing 0.3 mM NAT±5 mM L-Met were aliquoted into sterile glass vials (1 ml/vial). The vials were capped and placed on their side in the light box to maximize exposure to the light source. A control sample for each buffer condition was covered in foil and placed in the light box for the duration of exposure. For consistency with the other stress models, prior to HPLC analysis, the buffer solutions were diluted with Milli-Q H₂O to a final NAT concentration of 0.2 mM.

### Thermal Stress

Histidine and non-histidine buffers containing 1.0 mM NAT were aliquoted into sterile glass vials (5 ml/vial, 6 vials per buffer). Vials were stored in a dark box at the indicated temperatures during the stress and transferred to −70° C. for storage until analysis (timepoints taken monthly for five months). Initial time points for samples of each buffer solution were transferred immediately to −70° C. Prior to analysis, the samples were thawed and diluted with Milli-Q H₂O to a final NAT concentration of 0.2 mM.

### HPLC Analysis

NAT and NAT degradants were separated on an Agilent 1200 series HPLC or Waters H Class UPLC using an Agilent ZORBAX SB-C18 3.5 μm, 4.6×75 mm reverse phase column. Column temperature was held at 30±0.8° C. by a thermostat controller. The gradients used for HPLC and UPLC are listed in Tables 5 and 6, respectively (note: the shorter gradient on the UPLC was designed to accommodate earlier retention times and the column re-equilibration period was elongated due to the larger range of system pressure at a flow rate of 1.0 ml/min). NAT degradation products were detected at 240 nm. The standard bandwidth setting (8 nm for Agilent 1200 HPLC, 4.8 nm for Waters H-Class) was used for analysis on each instrument. The autosampler was maintained at 5±3° C. 10 nmol of NAT and/or NAT degradants (50 μl for most samples) was injected on to the column for analysis. The chromatograms were processed using Dionex Chromeleon software.

TABLE 5
Gradient for Agilent 1200 HPLC.

| | Mobile Phase A % | Mobile Phase B % |
|---|---|---|
| Time (min) | Water (0.1% formic acid) | MeCN (0.1% formic acid) |
| 0 | 98 | 2 |
| 2 | 98 | 2 |
| 16 | 70 | 30 |
| 18 | 70 | 30 |
| 18.1 | 10 | 90 |
| 22 | 10 | 90 |
| 22.1 | 98 | 2 |
| 26 | 98 | 2 |

TABLE 6
Gradient for Waters H-Class UPLC.

| | Mobile Phase A % | Mobile Phase B % |
|---|---|---|
| Time (min) | Water (0.1% formic acid) | MeCN (0.1% formic acid) |
| 0 | 98 | 2 |
| 2 | 98 | 2 |
| 14 | 74 | 26 |
| 16 | 74 | 26 |
| 16.5 | 10 | 90 |
| 19.5 | 10 | 90 |
| 20 | 98 | 2 |
| 30 | 98 | 2 |

### Analysis of Samples by LC/MS

LC/MS sample analysis was conducted using a Waters H-Class UPLC and the chromatography conditions described above, in tandem with a Thermo Scientific Orbitrap Mass spectrometer. Full scan accurate mass data were collected at a resolution of 15,000 in positive ion mode over a scan range of 50-800 m/z. MS2 was performed of the top three ions with dynamic exclusion disabled.

### Results

### Stressed Sample Panel Design

NAT stability was assessed following exposure to four different representative stresses: 1) Fenton chemistry (H₂O₂+Fe²⁺), which mimics the potential oxidation caused by iron leachables resulting from contact with stainless steel during pharmaceutical production, 2) AAPH stress, which mimics the alkyl peroxides produced by the degradation of polysorbate detergents, 3) International Conference on Harmonization (ICH) light stress (1.2 million lux hours, 200 w hr/m2), a harsh light stress used in the pharmaceutical industry to assess photostability, and 4) accelerated thermal stress to simulate long term degradation of biopharmaceuticals. The intensity of each stress was selected to be harsh compared to typical shelf life and manufacturing and of similar degradative strength to each other such that comparisons between changes induced by the different stress models could be made. These studies were performed formulations consistent with those typically used for mAbs. As histidine is known to be oxidatively active, both histidine-containing and non-histidine containing buffers were employed where relevant.

### Method Development

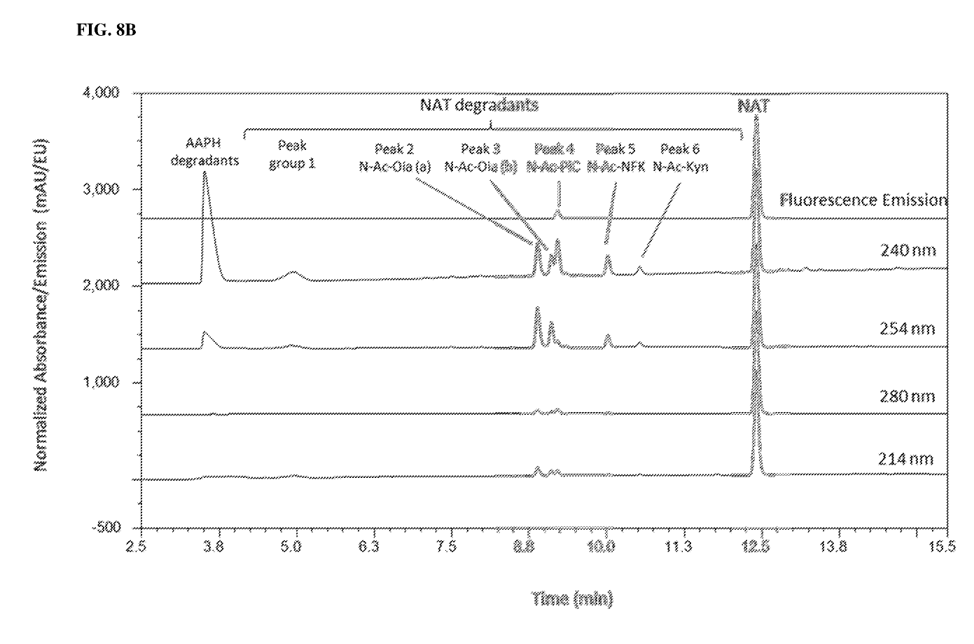

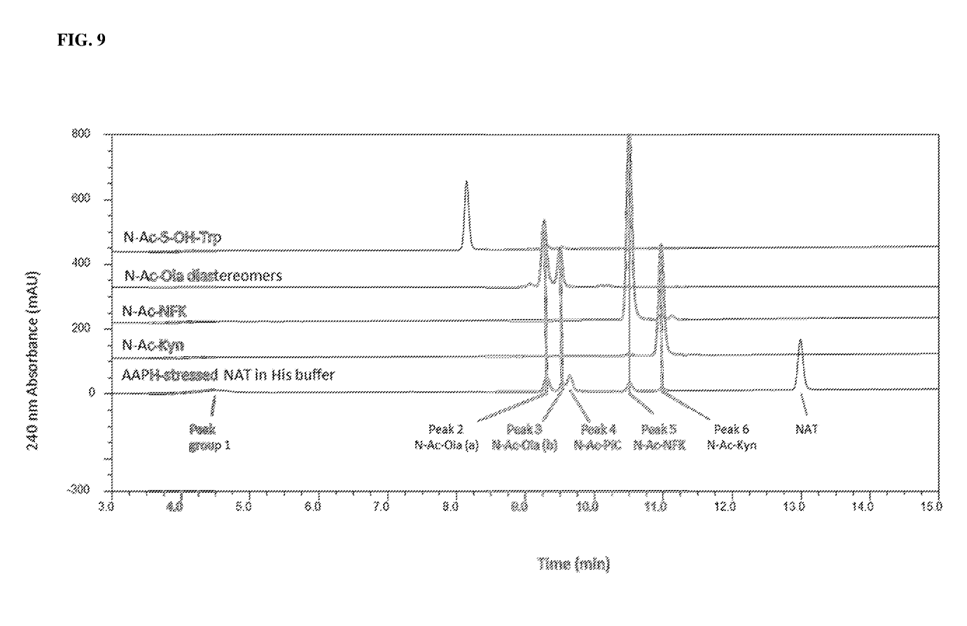

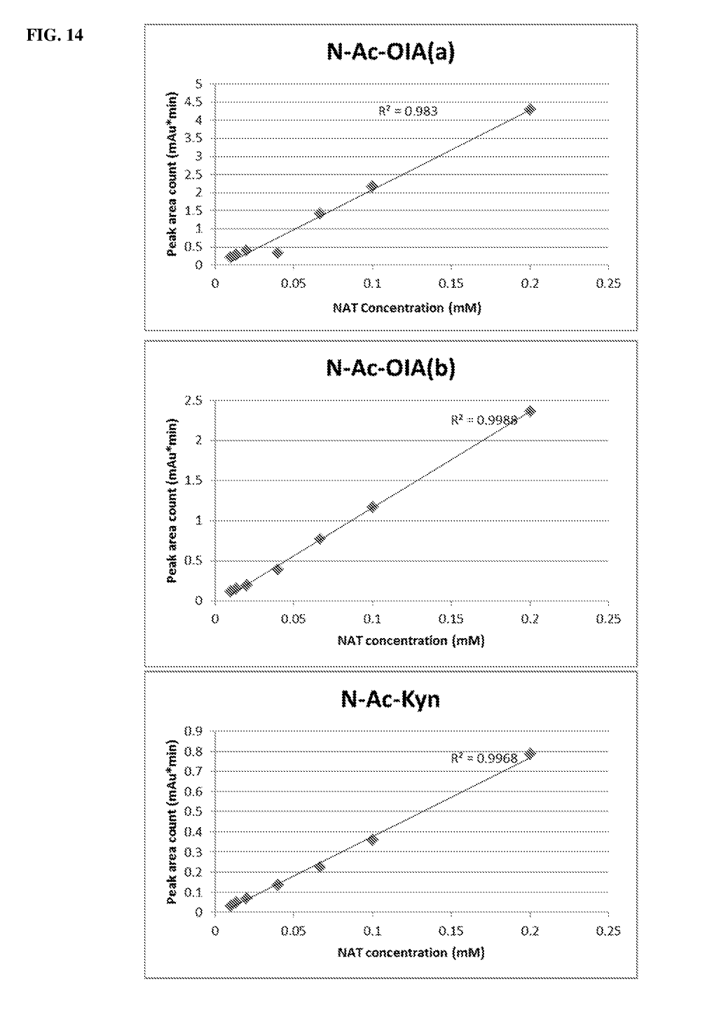

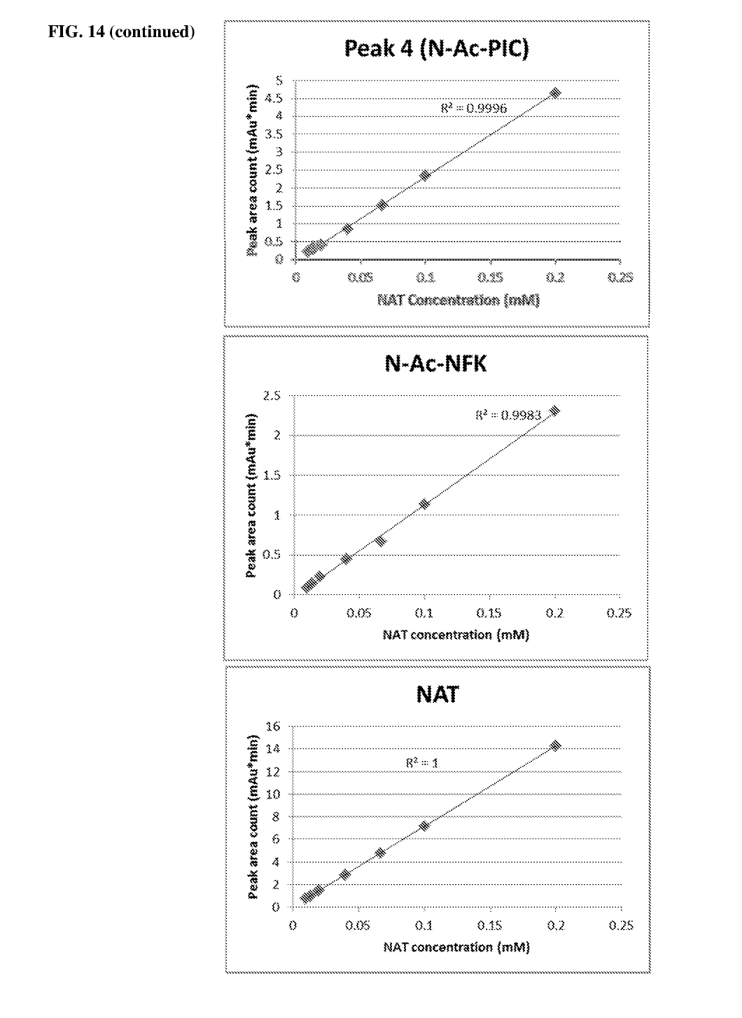

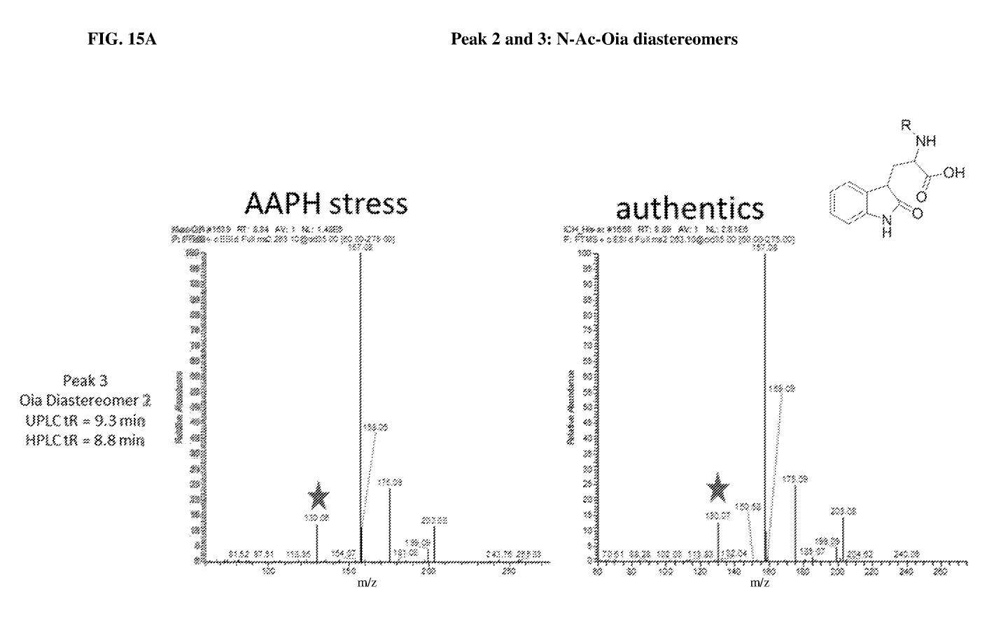

Reverse-phase chromatography using a C18 column was used to monitor NAT degradation. Gradient conditions were selected to assure suitable resolution of NAT and NAT degradants (FIG. 8A). Eluants were monitored at 240 nm, a wavelength selected to assure adequate sensitivity of low level species [some species were not detected at higher wavelengths (e.g. 280 nm) and signal: noise dropped at lower wavelengths (e.g. 214 nm), see FIG. 8B]. The final chromatography conditions provided linear responses through relevant ranges: 0.01-1.0 mM NAT (FIG. 13) and 1-20-fold dilution of degradants in an AAPH-stressed NAT sample (FIG. 14). Autosampler stability of NAT and the major degradants was established for up to 12 hours at 5° C. (data not shown).

Protein-containing samples were diluted in guanidine and the proteins removed via ultra-filtration (Amicon spin filters with a 30 kDa molecular weight cut-off). Incomplete recovery was observed in the absence of chaotropes for some mAbs, suggesting noncovalent interactions between NAT and protein can occur. NAT has previously been demonstrated to bind human serum albumin (HAS) (Anraku, M., et al., Biochim Biophys Acta, 2004. 1702(1): p. 9-17), but has not been reported to bind mAbs to date. Using the final sample preparation conditions, recovery of NAT was 94-99% for three tested antibody/antibody derivatives and recovery of NAT degradants was 98-100% (data not shown).

### Analysis of Stressed Sample Panel

Multiple NAT degradants, represented by six major new peaks and multiple minor peaks, were observed for all stress conditions (FIG. 8A, see FIG. 7 for NAT degradant structures). Total NAT degradation for each sample was calculated by comparing NAT peak area to the control sample for each stress model (Table 7). NAT degradation ranged from 3% (thermal stress, non-His buffer) to 83% (ICH light stress, His buffer).

TABLE 7
Model stress conditions and corresponding NAT degradation

| | Potential source during | | | % NAT |
|---|---|---|---|---|
| Model | DP manufacturing/ | Type of | | degradation ± |
| Stress | storage | stress | Buffer Type | 1SD |
| Fenton | stainless steel tanks | H₂O₂, | His | 40 ± 3 (n = 3) |
| | | hydroxyl | His + 5 mM Met | 15.2 ± 0.7 (n = 3) |
| | | radical | | |
| AAPH | surfactant degradation | alkyl | His | 39.8 ± 0.2 (n = 3) |
| | | peroxide | Non-His | 41.1 ± 0.7 (n = 3) |
| | | | His + 5 mM Met | 34.7 ± 0.1 (n = 3) |
| | | | Non-His + 5 mM Met | 32.9 ± 0.5 (n = 3) |
| ICH | light exposure | singlet | His | 83.4 ± 0.2 (n = 2) |
| Light | | oxygen, | Non-His | 28.0 ± 0.1 (n = 2) |
| | | H₂O₂, | His + 5 mM Met | 17.6 ± 0.7 (n = 2) |
| | | superoxide | Non-His + 5 mM Met | 18 ± 2 (n = 2) |
| Thermal | shelf life, shipping, | elevated | His | 11.1 (n = 1) |
| | processing | temperature | Non-His | 2.9 (n = 1) |
| | | (40° C., 5 | | |
| | | months) | | |

*Starred peaks represent peaks only observed under ICH light stress.

Approximately 30-40% NAT degradation was observed for the Fenton and AAPH stresses under all tested conditions and the level and distribution of degradants was generally independent of the presence of histidine in the buffer (FIG. 8A, see FIG. 7 for NAT degradant structures). NAT experienced greater buffer sensitivity (i.e. the difference between histidine and non-histidine formulations) while under ICH light stress (FIG. 8A). While stability of NAT in the non-histidine buffer under light stress led to quantitatively similar NAT degradation as the AAPH and Fenton stresses (28% vs. 33-41% loss), the distribution of degradants changed and new peaks were observed (see “*” indicated peaks, FIG. 8A). Significantly higher levels of degradation (>80%) were observed in the histidine buffer under light stress, resulting in elevated levels of the previously observed NAT degradants, along with new peaks (FIG. 8A).

Overall, a striking consistency between profiles in the degraded sample panel was observed, with the exception of minor peaks observed under ICH light stress conditions (“*” indicated peaks in FIG. 8A). This suggests that hydrogen peroxide/hydroxyl radical (Fenton stress) and alkyl peroxide (AAPH) may degrade NAT via a common pathway, whereas the reactive oxygen species (ROS) induced by UV light (H₂O₂, singlet oxygen, superoxide) may present additional degradation pathways. The observation that the presence of histidine increased NAT degradation under ICH light conditions is consistent with reports that histidine itself is photoreactive and could therefore increase ROS levels and types (Stroop, S. D. et al., J Pharm Sci, 2011. 100(12): p. 5142-55). Given the common NAT degradation profiles observed under these diverse stress conditions tested, it is likely that any NAT degradation in drug products would lead to the production of these same species.

### Degradant Identification

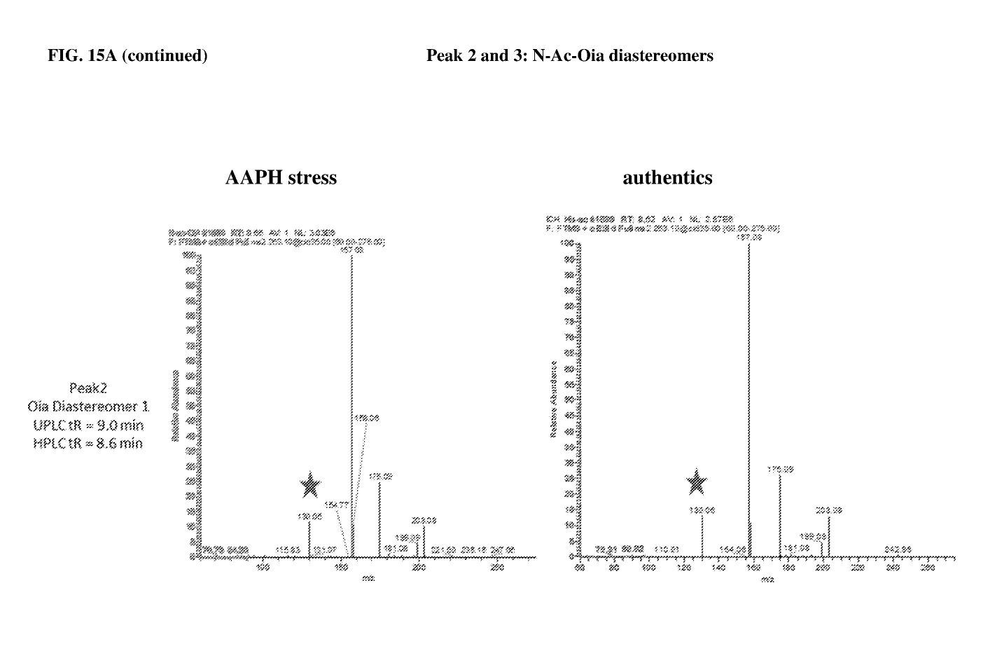

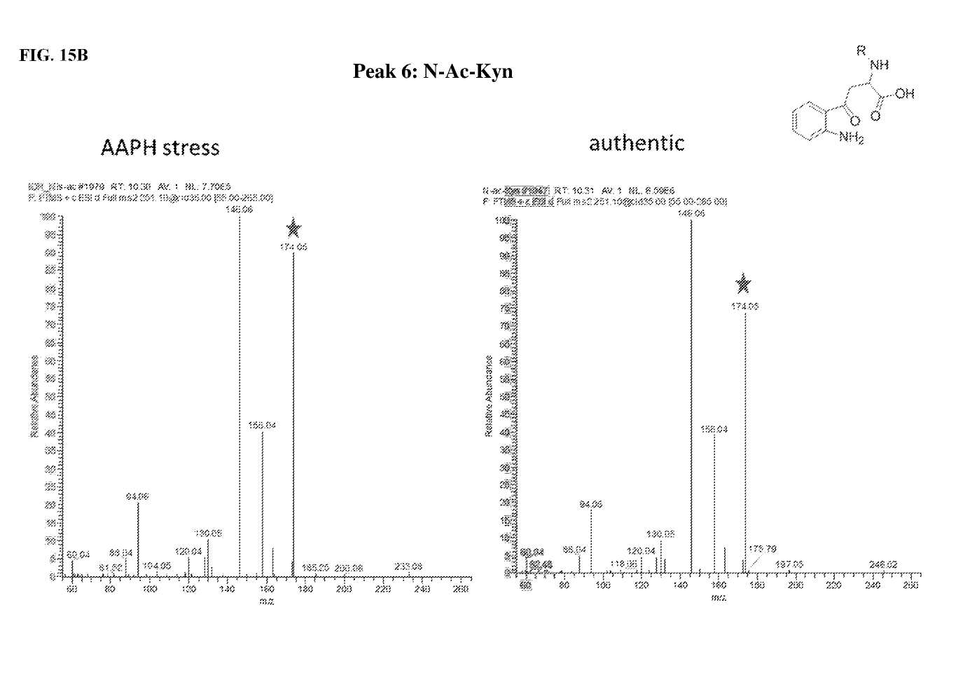

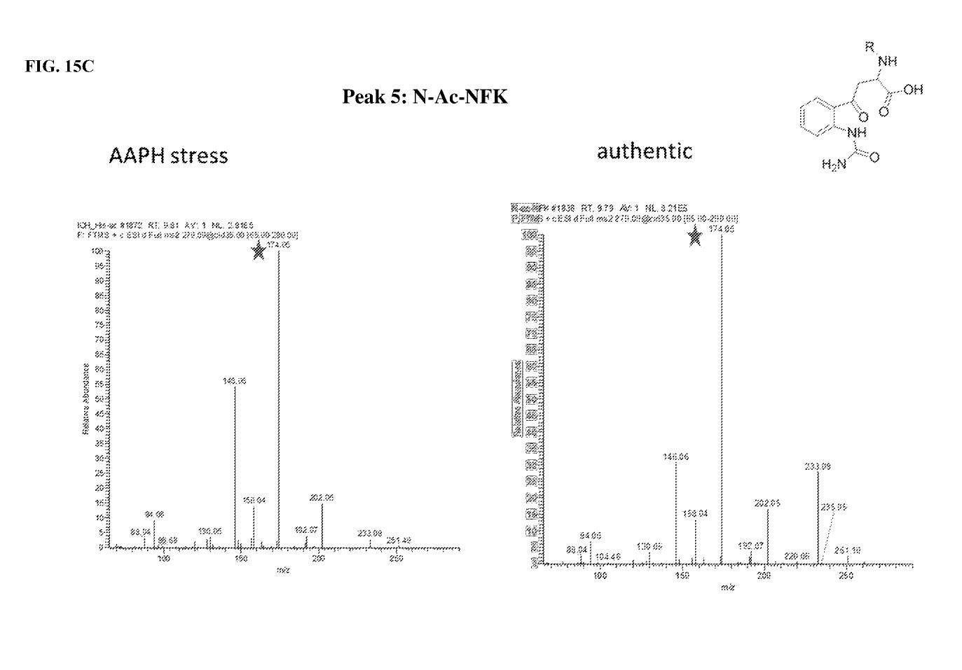

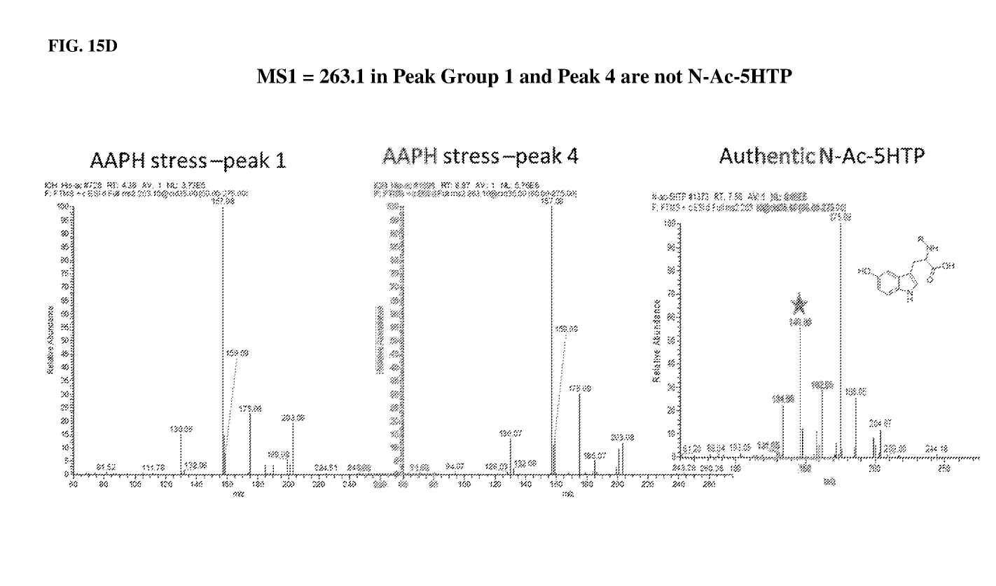

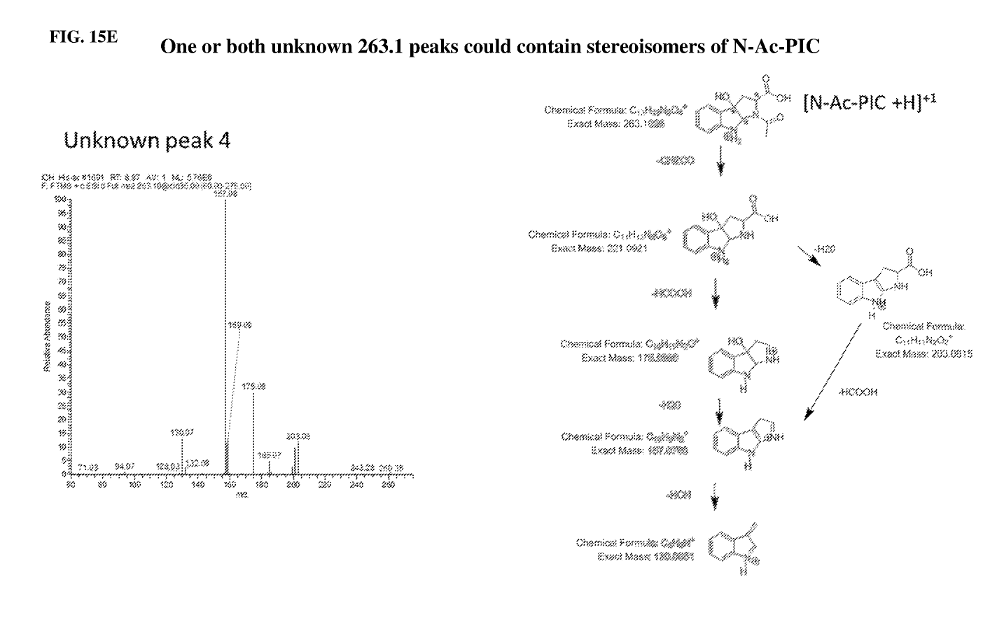

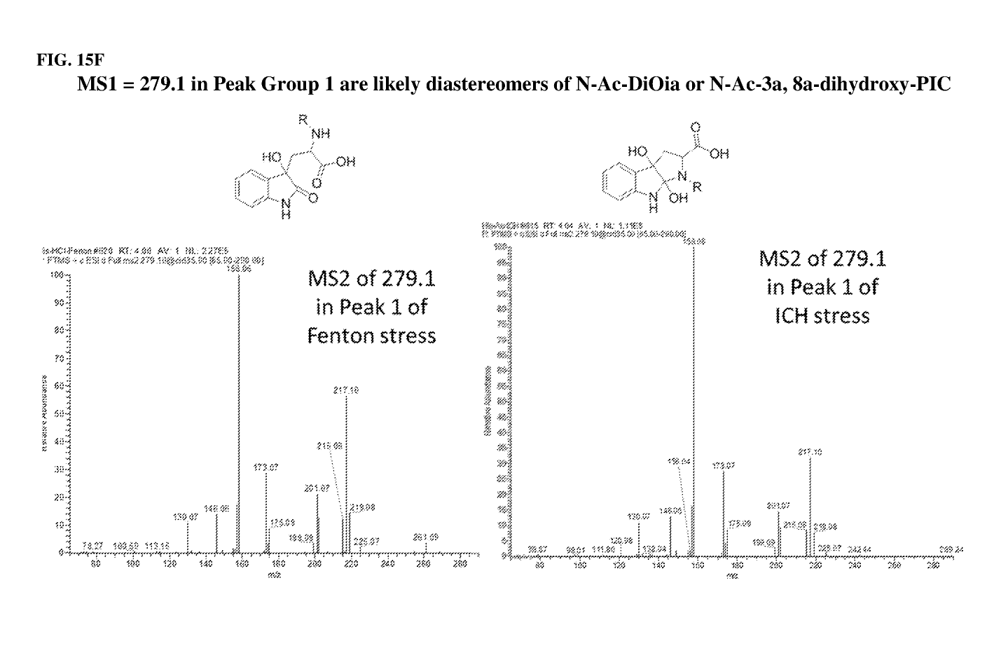

Next, the identities of the degraded NAT degradant species were explored using LC/MS/MS. The molecular ions for major peaks are listed in Table 8 (a complete list of all peaks that exhibited adequate signal intensity by LC/MS is included in Table 9). Major peaks 2, 3, and 4 had an m/z of 263.1 (NAT+16), consistent with a single oxidation event of NAT. As major peaks 2 and 3 had similar MS1 and MS2 spectra (FIG. 15A) and consistent ratios across all stresses and absorbance wavelengths monitored (FIG. 8B), these peaks were tentatively assigned as the interconverting diastereomers of N-Ac-Oia 4b (see FIG. 7 for structure). The analogous Trp species has been reported after hydrogen peroxide treatment of tryptophan (Simat, T. J. and H. Steinhart, J Agric Food Chem, 1998. 46(2): p. 490-498). This assignment is further supported by the observation of an MS2 ion at 130.1, previously reported to be indicative of oxyindolylalanine (Oia)-containing peptides (Todorovski, T. et al., J Mass Spectrom, 2011. 46(10): p. 1030-8.) (FIG. 15A).

TABLE 8
NAT degradant identification

| | Retention time | | Expected | |
|---|---|---|---|---|
| Peak # | UPLC (mm) | Identity | m/z | Observed m/z |
| Group 1 | 4.5-5.4 min | includes stereoisomers of | 279.098, | ICH: 279.096, 263.102 |
| | | N-Ac-PIC 2b | 263.103 | Fenton: 279.096, |
| | | and N-Ac-2a,8a,-dihydroxy- | | 263.102 |
| | | PIC 3b (tentative) | | AAPH: 263.102 |
| 2 | 9.04 | diastereomer 1, N-Ac-Oia 4b | 263.103 | 263.102 |
| 3 | 9.25 | diastereomer 2, N-Ac-Oia 4b | 263.103 | 263.102 |
| 4 | 9.36 | stereoisomer(s) of N-Ac-PIC | 263.103 | 263.102 |
| | | 2b (tentative) | | |
| 5 | 10.16 | N-Ac-NFK 7b | 279.098 | 279.098 |
| 6 | 10.63 | N-Ac-Kyn 6b | 251.103 | 251.103 |
| 7 | 12.53 | NAT 1b | 247.108 | 247.108 |

TABLE 9
Identities of NAT degradant species

| UPLC Ret. | | | Expected | Observed |
|---|---|---|---|---|
| Time (min) | Peak identifier | Identity | m/z | m/z |
| 4.5-5.4 | peak group 1 | group, including stereoisomers of | 279.098, | 279.096, |
| | | N-Ac-PIC 2b and N-Ac-2a,8a,- | 263.103 | 263.102 |
| | | dihydroxy-PIC 3b (tentative) | | |
| 5.8 | ICH light stress | unknown --NAT + double | N/A | 279.096 |
| | minor peak | oxidation | | |
| 9.04 | peak 2 | diastereomer 1, N-Ac-Oia 4b | 263.103 | 263.102 |
| 9.25 | peak 3 | diastereomer 2, N-Ac-Oia 4b | 263.103 | 263.102 |
| 9.36 | peak 4 | stereoisomer(s) of N-Ac-PIC 2b | 263.103 | 263.102 |
| | | (tentative) | | |
| 9.50 | ICH light stress | unknown --NAT + double | N/A | 279.096 |
| | minor peak | oxidation | | |
| 10.16 | peak 5 | N-Ac-NFK 7b | 279.098 | 279.098 |
| 10.63 | peak 6 | N-Ac-Kyn 6b | 251.103 | 251.103 |
| 10.72 | ICH light stress | unknown --NAT + double | N/A | 279.096 |
| | minor peak | oxidation | | |
| 12.53 | peak 2 | NAT 1b | 247.108 | 247.108 |
| 13.43 | ICH light stress | unknown --NAT + double | N/A | 279.096 |
| | minor peak | oxidation | | |

Major peaks 5 and 6 had m/z of 279.10 (NAT+32) and 251.10 (NAT+4) respectively, had no or weak absorbance at 280 nm (FIG. 8B). These properties, suggesting loss of indole ring, are consistent with two of the major known physiological degradants of Trp, NFK (7a, Trp+32) and Kyn (6a, Trp+4) (Dreaden K., et al, PLoS One, 2012. 7(7): p. e42220) (see FIG. 7 for structures). To assess whether these species represented the corresponding N-acetylated versions, N-Ac-NFK 7b and N-Ac-Kyn 6b (see FIG. 7 for structures), collision induced dissociation was used to generate MS2 spectra for both species. Each displayed a strong signal at m/z=174.1 (FIG. 15B and FIG. 15C), previously reported as characteristic of kynurenines (Todorovski, T. et al., J Mass Spectrom, 2011. 46(10): p. 1030-8). Based on this information these species were tentatively assigned as N-Ac-NFK 7b and N-Ac-Kyn 6b, respectively (see FIG. 7 for structures).

To confirm the identities of these species, authentic standards of N-Ac-Oia 4b, N-Ac-NFK 7b, and N-Ac-Kyn 6b were synthesized using synthetic procedures described above. Both the chromatographic and MS2 profiles of peaks in stressed NAT samples aligned with those of the authentic samples (FIG. 9 and FIGS. 15A-15C) lending additional support to the identification of these peaks.

Given that 5-OH-Trp 8a is the major physiological catabolite of Trp, a synthetic standard of N-Ac-5-OH-Trp 8b (see FIG. 7 for structures) was also prepared to assess if the species was along a major degradation pathway for NAT. Analysis of the authentic N-Ac-5-OH-Trp 8b standard by LC-MS/MS indicated that the compound was not present in any significant amount in any of the stressed NAT samples, as neither the retention time nor the mass spectrometry data was consistent with the observed NAT degradants (FIG. 9 and FIG. 15D). The MS2 fragment ion 146.1, derived from Trp derivatives that have been oxidized on the benzene portion of the indole ring (Todorovski, T. et al., J Mass Spectrom, 2011. 46(10): p. 1030-8) was not observed in any of the singly oxidized NAT degradant species, suggesting minimal levels of hydroxylation occurs on the 4, 5, 6, or 7 position of the indole ring during NAT oxidation (FIG. 15A and FIG. 15D).

The singly oxidized NAT species in peak group 1 and peak 4 were tentatively identified as the stereoisomers of N-Ac-PIC 2b (see FIG. 7 for structure) and the doubly oxidized NAT species in peak group 1 were similarly tentatively assigned as the stereoisomers of N-Ac-3a, 8a-dihydroxy PIC 3b, respectively. These molecules were the only NAT degradants reported upon extended thermal stress (3 years at 25° C.) of a NAT-containing HSA formulation (Fang, L. et al., Chromatogr A, 2011. 1218(41): p. 7316-24) and the MS2 fragmentation patterns observed in our studies are consistent with that report (FIG. 15E and FIG. 15F). Furthermore, peak 4 is the only NAT degradant observed using fluorescence detection in these studies (FIG. 8B), consistent with reports that H, 1,2,3,3a,8,8a-hexahydro-3a-hydroxypyrrolo [2,3-b]-indole 2-carboxylic acid (PIC) 2a is one of the only common Trp degradants that is fluorescent (Simat, T. J. et al., J Agric Food Chem, 1998. 46(2):490-498). As synthetic standards for these species were not prepared, these identifications cannot be conclusively determined and it remains possible that the doubly oxidized N-Ac-dioxyindolylalanine (N-Ac-DiOia) is also present in the incompletely resolved Peak group 1. The peak assignments are summarized in Table 8.

The NAT degradants observed in these studies (N-Ac-PIC, N-Ac-Oia, N-Ac-NFK, N-Ac-Kyn, and N-Ac-2a,8a,-dihydroxy-PIC) are largely consistent with those reported by Simat et al. for free tryptophan oxidized by treatment with hydrogen peroxide (PIC, Oia, NFK, Kyn, DiOia, and 5-OH-Trp) (Simat, T. J. J Agric Food Chem, 1998. 46(2): p. 490-498). Definitive identifications of tryptophan degradants in peptides and proteins are limited (as the isobaric nature of many tryptophan derivatives complicates identification of degradants at the peptide and protein level, and isolation of the individual residues can lead to decomposition), but the peptide/protein literature is similarly consistent with the NAT studies (Simat, T. J. et al., J Agric Food Chem, 1998. 46(2): p. 490-498; Fedorova, M., et al., Proteomics, 2010. 10(14): p. 2692-700; Li, Y., et al., Anal Chem, 2014. 86(14): p. 6850-7; Ronsein, G. E., et al., J Am Soc Mass Spectrom, 2009. 20(2): p. 188-97). One disparity of note is 5-OH-Trp—this is a major degradant of Trp in vivo (via the tryptophan hydroxylase pathway) and was observed in the study by Simat el al for free Trp—however, it was observed only at trace levels upon oxidation of the tripeptide Ala-Trp-Ala under the same hydrogen peroxide conditions, was not definitely identified in the peptide and protein literature surveyed, and was not observed in our studies on NAT.

Taken together, this suggests that Trp derivatives containing amidated N-termini (as in NAT and in peptides/proteins) may be less susceptible to oxidation at the 5 position relative to free Trp under non-enzymatic conditions.

### Effect of Other Excipients and Protein on NAT Degradation

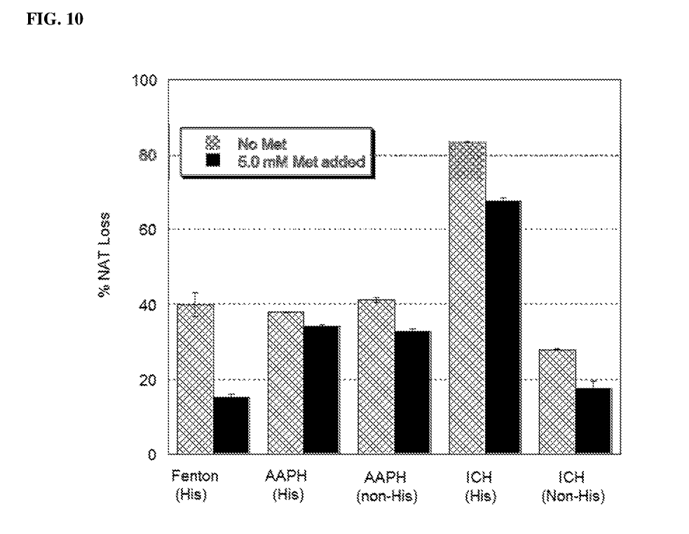

Next, the impact of other excipients on NAT degradation was assessed. Of particular interest is the presence of Met, another antioxidant commonly added to drug product formulations as an antioxidant. In general, the inclusion of 5 mM Met in the buffer formulations led to an overall decrease in total NAT oxidation (Table 7, FIG. 10), which is consistent with the hypothesis that thioether moiety in Met can serve as an oxidative sink. The impact of Met on NAT stability varied between the oxidation models: Met made a modest improvement to NAT stability in the AAPH model (4-8% total NAT loss, depending on buffer), a slightly better improvement under ICH light stress conditions (10-16%), and a significant improvement in the Fenton model (25%) (Table 8). The significant decrease in NAT oxidation when formulated with Met in the Fenton conditions may be due to the Met quenching the hydrogen peroxide (Ji, J. A., et al., J Pharm Sci, 2009. 98(12): p. 4485-500). The addition of Met appeared to have not altered the oxidation mechanism in each of the model systems, as the distribution of the major species observed in Met-formulated stress samples was unchanged from those formulated without Met (data not shown).

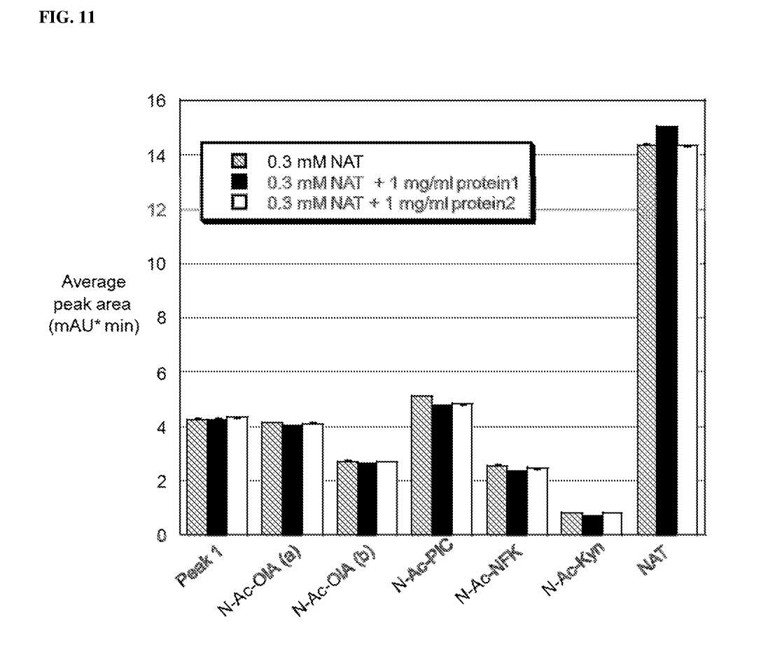

The impact of low concentrations of protein on AAPH-induced NAT degradation was also analyzed. Two antibodies (protein1 and protein2) were diluted to 1.0 mg/ml in buffer and formulated with 0.3 mM NAT (˜45:1 mol NAT:mol protein). Upon AAPH stress, the level of NAT degradation was largely consistent between protein-containing and protein-free solutions (˜40% loss in NAT peak area), as was the distribution of oxidant species (FIG. 11). These results suggest that the presence of low levels of protein does not inherently impact NAT degradation levels/pathways under the tested oxidative (alkyl peroxide-induced) stress conditions.

### Real Time Stability of NAT in Drug Product Formulations

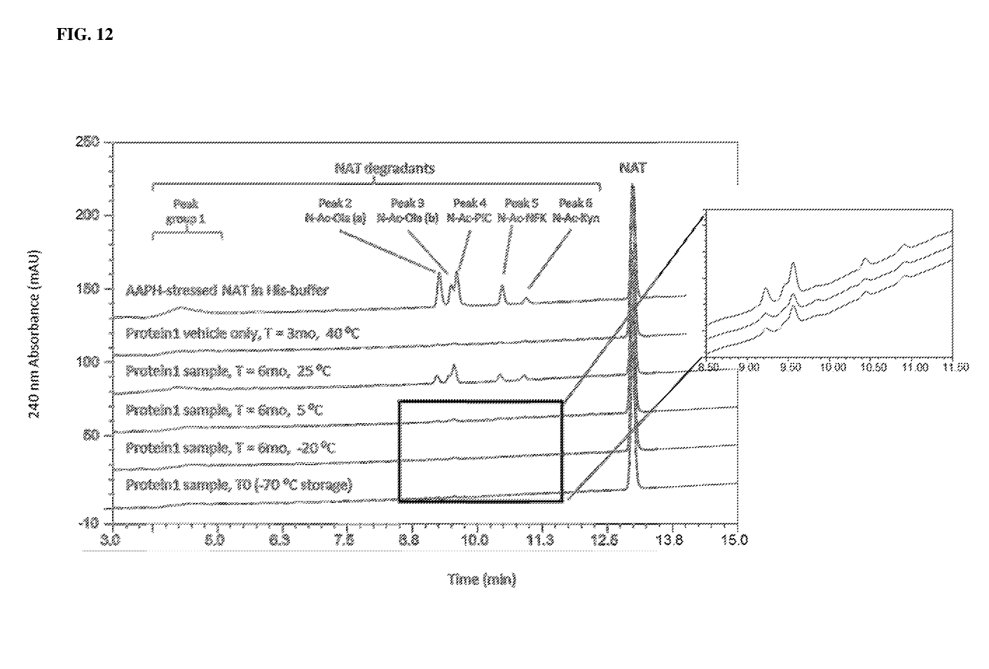

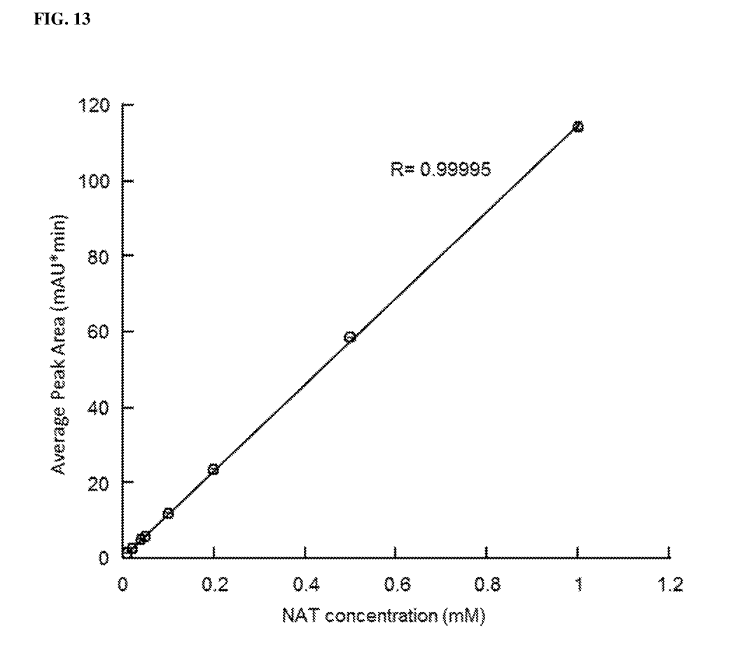

With this model experience in hand, the amount of NAT oxidation expected to occur in the manufacturing and storage of mAbs was explored next. FIG. 12 illustrates the comparison of the AAPH model stress system with an antibody at 150 mg/ml (1.0 mM) co-formulated with NAT, Met, and other excipients typical of mAb formulations. Results are shown for the initial time point, at −20° C. and 5° C. for six months (representative of typical storage conditions), and 25° C. for 6 months (representative of accelerated stability conditions). Oxidation levels were negligible directly after manufacturing and under the typical storage conditions tested. After six months at 25° C., some degradation was observed (total NAT loss=16.8%). Of interest, the corresponding vehicle showed significantly lower NAT degradation—while the protein containing sample had 7.5% NAT loss after 3 months at 25° C., the corresponding vehicle showed only 1% loss of NAT. Even at higher temperatures (FIG. 12) the vehicle showed minimal NAT degradation suggesting that the presence of high concentrations of protein may increase NAT degradation under accelerated thermal conditions in some cases. The five major species present in the accelerated stability sample corresponded with the major species identified in the stress models (FIG. 12), suggesting the models faithfully recapitulate the NAT degradation pathways in drug product samples.

To summarize, using a chromatographic method for assessing the stability of N-Ac-tryptophan, an antioxidant known to provide protection against oxidative stress to Trp residues in protein therapeutics, NAT was shown to degrade into a set of common degradants—including N-Ac-Oia, N-Ac-PIC, N-Ac-Kyn, and N-Ac-NFK (see FIG. 7 for structures)—largely independent of stress type under diverse stress conditions and in the different model formulations, a finding that has not previously been reported. These degradants are generally consistent with the literature on Trp oxidation, with the exception that NAT degradation in the studied stress models did not lead to production of the N-acetylated version of 5-hydroxytryptophan, the most common physiologic Trp degradant. Without being bound by theory, this suggests that under non-enzymatic conditions, NAT (and perhaps, by extension, Trp residues in proteins) does not degrade via the same intermediates as Trp catabolism. In fact, without being bound by theory, the data suggest that oxidation of NAT occurs primarily on the 2 and 3 positions of the indole ring.

1. A method of reducing oxidation of a polypeptide in an aqueous formulation comprising adding an amount of N-acetyltryptophan to the formulation that prevents oxidation of the polypeptide, wherein the polypeptide comprises at least one tryptophan residue with a solvent-accessible surface area (SASA) of greater than about 80 Å².

2. A method of reducing oxidation of a polypeptide in an aqueous formulation comprising adding an amount of N-acetyltryptophan to the formulation that prevents oxidation of the polypeptide, wherein the polypeptide comprises at least one tryptophan residue with a solvent-accessible surface area (SASA) of greater than about 30%.

3. A method of reducing oxidation of a polypeptide in an aqueous formulation comprising determining the SASA values of tryptophan residues in the polypeptide and adding an amount of N-acetyltryptophan to the formulation that prevents oxidation of the polypeptide if at least one tryptophan residue has a solvent-accessible surface area (SASA) of greater than about 80 Å².

4. The method of claim 3, wherein the SASA value of the tryptophan residues in calculated by molecular dynamic simulation.

5. The method of claim 1, wherein the N-acetyltryptophan is added to the formulation to a concentration of about 0.1 mM to about 5 mM, about 0.1 mM to about 1 mM, or about 0.3 mM.

6-7. (canceled)

8. The method of claim 1, wherein the oxidation of the polypeptide is reduced by about 50%, 75%, 80%, 85%, 90%, 95% or 99%.

9. The method of claim 1, wherein the formulation is stable at about 2° C. to about 8° C. for about 1095 days.

10. The method of claim 1, wherein the protein concentration in the formulation is about 1 mg/mL to about 250 mg/mL.

11. The method of claim 1, wherein the formulation has a pH of about 4.5 to about 7.0.

12. The method of claim 1, wherein the formulation further comprises one or more excipients selected from the group consisting of a stabilizer, a buffer, a surfactant, and a tonicity agent.

13. The method of claim 1, wherein the formulation is a pharmaceutical formulation suitable for administration to a subject.

14. (canceled)

15. The method of claim 1, wherein the protein is a polyclonal antibody, a monoclonal antibody, a humanized antibody, a human antibody, a chimeric antibody, a multispecific antibody, or an antibody fragment.

16. A liquid formulation comprising a polypeptide and an amount of N-acetyltryptophan to prevent oxidation of the polypeptide, wherein the polypeptide has at least one tryptophan residue with a SASA of greater than about 80 Å².

17. The liquid formulation of claim 16, wherein the N-acetyltryptophan is added to the formulation to a concentration of about 0.1 mM to about 5 mM, about 0.1 mM to about 1 mM, or about 0.3 mM.

18-19. (canceled)

20. The liquid formulation of claim 16, wherein the oxidation of the polypeptide is reduced by about 50%, 75%, 80%, 85%, 90%, 95% or 99%.

21. The liquid formulation of claim 16, wherein the formulation is stable at about 2° C. to about 8° C. for about 1065 days.

22. The liquid formulation of claim 16, wherein the protein concentration in the formulation is about 1 mg/mL to about 250 mg/mL.

23. The liquid formulation of claim 16, wherein the formulation has a pH of about 4.5 to about 7.0.

24. The liquid formulation of claim 16, wherein the formulation further comprises one or more excipients selected from the group consisting of a stabilizer, a buffer, a surfactant, and a tonicity agent.

25. The liquid formulation of claim 16, wherein the formulation is a pharmaceutical formulation suitable for administration to a subject.

26. (canceled)

27. The liquid formulation of claim 16, wherein the protein is a polyclonal antibody, a monoclonal antibody, a humanized antibody, a human antibody, a chimeric antibody, a multispecific antibody, or an antibody fragment.

28. A method for screening a formulation for reduced oxidation of a polypeptide wherein the polypeptide comprises at least one tryptophan residue with a SASA of greater than about 80 Å², the method comprising
adding an amount of N-acetyltryptophan to an aqueous composition comprising the polypeptide,
adding 2,2′-azobis (2-aminopropane) dihydrochloride (AAPH) to the composition,
incubating the composition comprising the polypeptide, N-acetyltryptophan and AAPH for about 14 hours at about 40° C.,
measuring the polypeptide for oxidation of tryptophan residues in the polypeptide,
wherein a formulation comprising an amount of N-acetyltryptophan that results in no more than about 20% oxidation of tryptophan residues of the polypeptide is a suitable formulation for reduced oxidation of the polypeptide.

29. A method for screening a formulation for reduced oxidation of a polypeptide comprising
determining the SASA values of tryptophan residues in the polypeptide, wherein a tryptophan residue with a SASA of greater than about 80 Å², is subject to oxidation,
adding an amount of N-acetyltryptophan to an aqueous composition comprising the polypeptide,
adding 2,2′-azobis (2-aminopropane) dihydrochloride (AAPH) to the composition,
incubating the composition comprising the polypeptide, N-acetyltryptophan and AAPH for about 14 hours at about 40° C.,
measuring the polypeptide for oxidation of tryptophan residues in the polypeptide,
wherein a formulation comprising an amount of N-acetyltryptophan that results in no more than about 20% oxidation of tryptophan residues of the polypeptide is a suitable formulation for reduced oxidation of the polypeptide.

30. The method of claim 29, wherein the SASA value of the tryptophan residues in calculated by molecular dynamic simulation.

31. A kit comprising the liquid formulation of claim 16.

32. An article of manufacture comprising the liquid formulation of claim 16.

33. A method to determine if a polypeptide in a liquid formulation comprises a tryptophan residue susceptible to oxidation, the method comprising calculating one or more molecule descriptors based on the amino acid sequence of the polypeptide for each tryptophan residue in the polypeptide and applying the one or more molecule descriptors to a machine learning algorithm trained on the one or more molecule descriptors to predict tryptophan oxidation, wherein the molecule descriptors include one or more of the following:
a) number of aspartic acid sidechain oxygens within 7 Å of tryptophan delta carbon,
b) sidechain solvent accessible surface area (SASA),
c) delta carbon SASA,
d) total positive charge within 7 Å of tryptophan delta carbon,
e) backbone SASA,
f) tryptophan sidechain angles,
g) packing density within 7 Å of tryptophan delta carbon,
h) tryptophan backbone angles,
i) SASA of pseudo-pi orbitals,
j) backbone flexibility, or
k) total negative charge within 7 Å of tryptophan delta carbon.

34-40. (canceled)

41. A method to reduce oxidation of a polypeptide, comprising identifying tryptophan residues susceptible to oxidation according to the method of claim 33 and introducing an amino acid substitution in the polypeptide to replace one or more tryptophan residues susceptible to oxidation with amino acid residues that are not subject to oxidation.

42-43. (canceled)

44. A method to reduce oxidation of a polypeptide in an aqueous formulation, comprising determining the presence of one or more tryptophan residues in the polypeptide susceptible to oxidation according to the method of claim 33, and adding an effective amount of an anti-oxidation agent to the aqueous formulation comprising a polypeptide having a one or more tryptophan residues susceptible to oxidation.

45. (canceled)

46. The method of claim 44, wherein the anti-oxidation agent is N-acetyltryptophan.

47.-57. (canceled)

58. A liquid formulation comprising a polypeptide and an amount of N-acetyltryptophan to prevent oxidation of the polypeptide, wherein the polypeptide has at least one tryptophan residue susceptible to oxidation as measured by the method of claim 33.

59.-69. (canceled)

70. A method for screening a formulation for reduced oxidation of a polypeptide wherein the polypeptide comprises at least one tryptophan susceptible to oxidation identified by the method of claim 33, the method comprising
adding an amount of N-acetyltryptophan to an aqueous composition comprising the polypeptide,
adding 2,2′-azobis (2-aminopropane) dihydrochloride (AAPH) to the composition,
incubating the composition comprising the polypeptide, N-acetyltryptophan and AAPH for about 14 hours at about 40° C.,
measuring the polypeptide for oxidation of tryptophan residues in the polypeptide,
wherein a formulation comprising an amount of N-acetyltryptophan that results in no more than about 20% oxidation of tryptophan residues of the polypeptide is a suitable formulation for reduced oxidation of the polypeptide.

71. A method for screening a formulation for reduced oxidation of a polypeptide comprising
a) identifying a polypeptide comprising one or more tryptophan residues susceptible to oxidation by the method of claim 33,
b) adding an amount of N-acetyltryptophan to an aqueous composition comprising the polypeptide identified in step a),
c) adding 2,2′-azobis (2-aminopropane) dihydrochloride (AAPH) to the composition,
d) incubating the composition comprising the polypeptide, N-acetyltryptophan and AAPH for about 14 hours at about 40° C.,
e) measuring the polypeptide for oxidation of tryptophan residues in the polypeptide,
wherein a formulation comprising an amount of N-acetyltryptophan that results in no more than about 20% oxidation of tryptophan residues of the polypeptide is a suitable formulation for reduced oxidation of the polypeptide.

72. A kit or an article of manufacture comprising the liquid formulation of claim 58.

73. (canceled)

74. A method for measuring N-acetyl tryptophan (NAT) degradation in a composition comprising N-acetyl tryptophan, the method comprising
a) applying the composition to a reverse phase chromatography material, wherein the composition is loaded onto the chromatography material equilibrated in a solution comprising a mobile phase A and a mobile phase B, wherein mobile phase A comprises acid in water and mobile phase B comprises acid in acetonitrile,
b) eluting the composition from the reverse phase chromatography material with a solution comprising mobile phase A and mobile phase B wherein the ratio of mobile phase B to mobile phase A is increased compared to step a), wherein NAT degradants elute from the chromatography separately from intact NAT,
c) quantifying the NAT degradants and the intact NAT.

75-98. (canceled)

99. A method for measuring N-acetyl tryptophan (NAT) degradation in a composition comprising N-acetyl tryptophan and a polypeptide, the method comprising
a) diluting the composition with about 8 M guanidine,
b) removing the polypeptide from the composition,
c) applying the composition to a reverse phase chromatography material, wherein the composition is loaded onto the chromatography material equilibrated in a solution comprising a mobile phase A and a mobile phase B, wherein mobile phase A comprises acid in water and mobile phase B comprises acid in acetonitrile,
d) eluting the composition from the reverse phase chromatography material with a solution comprising mobile phase A and mobile phase B wherein the ratio of mobile phase B to mobile phase A is increased compared to step a), wherein NAT degradants elute from the chromatography separately from intact NAT,
e) quantifying the NAT degradants and the intact NAT.

100-134. (canceled)

135. A method to monitor degradation of NAT in a composition comprising measuring the degradation of NAT in a sample of the composition according to the methods of claim 74.

136. (canceled)

137. A quality assay for a pharmaceutical composition, the quality assay comprising measuring degradation of NAT in a sample of the pharmaceutical composition according to the methods of claim 74, wherein the amount of NAT degradants measured in the composition determines if the pharmaceutical composition is suitable for administration to an animal.
